SEQUENCE LISTING (28 sequences)

SEQ ID NO: 1 - protein, 241 aa, Artificial sequence
  synthetic
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 2 - protein, 124 aa, Artificial sequence
  synthetic
MAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 3 - protein, 120 aa, Artificial sequence
  synthetic
QVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSS

SEQ ID NO: 4 - protein, 5 aa, Artificial sequence
  synthetic
LKEDI

SEQ ID NO: 5 - protein, 13 aa, Artificial sequence
  synthetic
FPLKMRMERQGAP

SEQ ID NO: 6 - protein, 4 aa, Artificial sequence
  synthetic
YNSH

SEQ ID NO: 7 - protein, 381 aa, Artificial sequence
  synthetic
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPVQ

SEQ ID NO: 8 - protein, 353 aa, Artificial sequence
  synthetic
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTTLCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFC

SEQ ID NO: 9 - protein, 372 aa, Artificial sequence
  synthetic
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAA

SEQ ID NO: 10 - protein, 341 aa, Artificial sequence
  synthetic
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLC

SEQ ID NO: 11 - protein, 356 aa, Artificial sequence
  synthetic
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQG

SEQ ID NO: 12 - protein, 351 aa, Artificial sequence
  synthetic
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCLKELKE

SEQ ID NO: 13 - protein, 400 aa, Artificial sequence
  synthetic
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 14 - protein, 343 aa, Artificial sequence
  synthetic
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCFPLKMRMERQ

SEQ ID NO: 15 - protein, 24 aa, Artificial sequence
  synthetic
MKTIIALSYIFCLVFADYKDDDDA

SEQ ID NO: 16 - protein, 25 aa, Artificial sequence
  synthetic
MRPQILLLLALLTLGLADYKDDDDA

SEQ ID NO: 17 - protein, 778 aa, Artificial sequence
  synthetic
MKTIIALSYIFCLVFADYKDDDDAMDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPVQGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 18 - protein, 761 aa, Artificial sequence
  synthetic
MRPQILLLLALLTLGLADYKDDDDAMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTTLCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCFPLKMRMERQGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 19 - protein, 769 aa, Artificial sequence
  synthetic
MKTIIALSYIFCLVFADYKDDDDAMEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAAGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 20 - protein, 871 aa, Artificial sequence
  synthetic
MKTIIALSYIFCLVFADYKDDDDAMGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCFPLKMRMERQGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIQVQLQESGGGLVQAGGSLRLSCAASGSIFSINTMGWYRQAPGKQRELVAAIHSGGSTNYANSVKGRFTISRDNAANTVYLQMNSLKPEDTAVYYCNVKDYGAVLYEYDYWGQGTQVTVSSLKEMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 21 - protein, 752 aa, Artificial sequence
  synthetic
MKTIIALSYIFCLVFADYKDDDDAMDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 22 - protein, 748 aa, Artificial sequence
  synthetic
MKTIIALSYIFCLVFADYKDDDDAMDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCLKELKEGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 23 - protein, 798 aa, Artificial sequence
  synthetic
MKTIIALSYIFCLVFADYKDDDDAMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLPGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSSAAA

SEQ ID NO: 24 - protein, 740 aa, Artificial sequence
  synthetic
MKTIIALSYIFCLVFADYKDDDDAMEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCFPLKMRMERQGAPNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 25 - protein, 765 aa, Artificial sequence
  synthetic
MRPQILLLLALLTLGLADYKDDDDAMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTTLCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCFPLKMRMERQGAPYNSHNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLKEDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 26 - protein, 412 aa, Artificial sequence
  synthetic
RRKWIKAGHAVRAIGRLSSPVVSERMYPEDGVLKSEIKKGLRLKDGGHYAAEVKTTYKAKKPVQLPGAYIVDIKLDIVSHNEDYTIVEQCERAEGRHPTGGRDELYKGGTGGSLVSKGEEDNMAIIKEFMRFKVHMEGSVNGHEFEIEGEGEGRPYEAFQTAKLKVTKGGPLPFAWDILSPQFTYGSKAYIKHPADIPDYFKLSFPEGFRWERVMNFEDGGIIHVNQDSSLQDGVFIYKVKLRGTNFPPDGPVMQKKTMGWEATRDQLTEEQIAEFKEAFSLFDKDGDGTITTKELGTVLRSLGQNPTEAELQDMINEVDADGDGTFDFPEFLTMMARRMNDTDSEVEIREAFRVFDNDGNGYIGAAELRHVMTDLGEKLTDEEVDEMIRVADIDGDGQVNYEEFVQMMTAK

SEQ ID NO: 27 - protein, 950 aa, Artificial sequence
  synthetic
MKTIIALSYIFCLVFADYKDDDDAMDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPVQASMVDSSRRKWIKAGHAVRAIGRLSSPVVSERMYPEDGVLKSEIKKGLRLKDGGHYAAEVKTTYKAKKPVQLPGAYIVDIKLDIVSHNEDYTIVEQCERAEGRHPTGGRDELYKGGTGGSLVSKGEEDNMAIIKEFMRFKVHMEGSVNGHEFEIEGEGEGRPYEAFQTAKLKVTKGGPLPFAWDILSPQFTYGSKAYIKHPADIPDYFKLSFPEGFRWERVMNFEDGGIIHVNQDSSLQDGVFIYKVKLRGTNFPPDGPVMQKKTMGWEATRDQLTEEQIAEFKEAFSLFDKDGDGTITTKELGTVLRSLGQNPTEAELQDMINEVDADGDGTFDFPEFLTMMARRMNDTDSEVEIREAFRVFDNDGNGYIGAAELRHVMTDLGEKLTDEEVDEMIRVADIDGDGQVNYEEFVQMMTAKDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

SEQ ID NO: 28 - protein, 933 aa, Artificial sequence
  synthetic
MRPQILLLLALLTLGLADYKDDDDAMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTTLCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCFPLKMRMERQASMVDSSRRKWIKAGHAVRAIGRLSSPVVSERMYPEDGVLKSEIKKGLRLKDGGHYAAEVKTTYKAKKPVQLPGAYIVDIKLDIVSHNEDYTIVEQCERAEGRHPTGGRDELYKGGTGGSLVSKGEEDNMAIIKEFMRFKVHMEGSVNGHEFEIEGEGEGRPYEAFQTAKLKVTKGGPLPFAWDILSPQFTYGSKAYIKHPADIPDYFKLSFPEGFRWERVMNFEDGGIIHVNQDSSLQDGVFIYKVKLRGTNFPPDGPVMQKKTMGWEATRDQLTEEQIAEFKEAFSLFDKDGDGTITTKELGTVLRSLGQNPTEAELQDMINEVDADGDGTFDFPEFLTMMARRMNDTDSEVEIREAFRVFDNDGNGYIGAAELRHVMTDLGEKLTDEEVDEMIRVADIDGDGQVNYEEFVQMMTAKDIMAQVQLVESGGGLVRPGGSLRLSCVDSERTSYPMGWFRRAPGKEREFVASITWSGIDPTYADSVADRFTTSRDVANNTLYLQMNSLKHEDTAVYYCAARAPVGQSSSPYDYDYWGQGTQVTVSS

(19) United States
(12) Patent Application Publication
(10) Pub. No.: US 2024/0141017 A1
(43) Pub. Date: May 2, 2024

(54) FLUORESCENT BIOSENSORS AND METHODS OF USE FOR DETECTING CELL SIGNALING EVENTS

(71) Applicant: THE REGENTS OF THE UNIVERSITY OF MICHIGAN, Ann Arbor, MI (US)

(72) Inventors: Wenjing Wang, Ann Arbor, MI (US); Kayla Kroning, Ann Arbor, MI (US)

(21) Appl. No.: 18/278,676

(22) PCT Filed: Feb. 25, 2022
(86) PCT No.: PCT/US2022/017804

§ 371 Date: Aug. 24, 2023

Related U.S. Application Data
(60) Provisional application No. 63/154,006, filed on Feb. 26, 2021.

(51) Int. Cl. C07K 14/72; C07K 16/18; G01N 33/58

(52) U.S. Cl. CPC C07K 14/723; C07K 16/18; G01N 33/582; C07K 2317/569; C07K 2319/60

(57) ABSTRACT

The present disclosure relates to fluorescent biosensors and methods of use thereof. In particular, provided herein is a genetically encoded fluorescent indicator (GEFI) comprising a circular-permuted fluorescent protein (cpFP) and an inhibitory molecule bound to the cpFP. In the basal state, the inhibitory molecule prevents fluorescence from the circular-permuted fluorescent protein. Upon conformational change and/or cleavage of the bond between the inhibitory molecule and the cpFP, the cpFP is disinhibited, thereby permitting fluorescence from the cpFP. The biosensors described herein may be used in methods for detecting a variety of cell-signaling events.

### STATEMENT REGARDING RELATED APPLICATIONS

This application claims priority to U.S. Provisional Patent Application No. 63/154,006, filed Feb. 26, 2021, the entire contents of which are incorporated herein by reference for all purposes.

### FIELD

The present disclosure relates to fluorescent biosensors and methods of use thereof. In particular, provided herein is a genetically encoded fluorescent indicator (GEFI) comprising a circular-permuted fluorescent protein (cpFP) and an inhibitory molecule bound to the cpFP. In the basal state, the inhibitory molecule prevents fluorescence from the circular-permuted fluorescent protein. Upon conformational change and/or cleavage of the bond between the inhibitory molecule and the cpFP, the cpFP is disinhibited, thereby permitting fluorescence from the cpFP. The biosensors described herein may be used in methods for detecting a variety of cell-signaling events.

### BACKGROUND

Cell signaling and protein-protein interactions (PPIs) are essential in living systems. Due to their importance, many tools have been developed to detect cell signaling events. However, there is still a lack of genetically-encoded methods to detect G-protein coupled receptor (GPCR) signaling and protease cleavage events.

### SUMMARY

In some aspects, provided herein are genetically-encoded fluorescent indicators (GEFI). In one aspect, provided herein is a GEFI comprising a circularly-permuted fluorescent protein (cpFP), and an inhibitory molecule bound to the cpFP. In some embodiments, the inhibitory molecule inhibits fluorescence from the cpFP in the basal state. In some embodiments, cpFP fluorescence is disinhibited upon conformational change of the GEFI and/or disruption of the bond between the cpFP and the inhibitory molecule.

In some embodiments, the inhibitory molecule bound to the cpFP is a nanobody. For example, the nanobody may be Nb39. In some embodiments, the inhibitory molecule is bound to the cpFP by a linker. For example, the inhibitory molecule may be bound to the cpFP by the linker LKEDI (SEQ ID NO: 4).

In some embodiments, cpFP is bound to a protein. For example, the cpFP may be bound to a G-protein coupled receptor (GPCR). In some embodiments, the cpFP is bound to the C-terminal domain of the GPCR. In some embodiments, the GPCR is an opioid receptor. For example, the opioid receptor may be a mu-opioid receptor, a kappa-opioid receptor, a delta-opioid receptor, or a chimeric opioid receptor. For example, in some embodiments the opioid receptor is a kappa-opioid receptor comprising the amino acid sequence of SEQ ID NO: 7, SEQ ID NO: 11, or SEQ ID NO: 12. As another example, in some embodiments the opioid receptor is a chimeric opioid receptor comprising the amino acid sequence of SEQ ID NO: 8.

In some embodiments, the cpFP is bound to the protein by a linker. For example, the cpFP may be bound to the protein by a linker FPLKMRMERQGAP (SEQ ID NO: 5). As another example, the cpFP may be bound to the protein by the linker GAP.

In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 17. In some embodiments, the GEFI comprises the amino acid sequence of SEQ ID NO: 17. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 21. In some embodiments, the GEFI comprises the amino acid sequence of SEQ ID NO: 21. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 18. In some embodiments, the GEFI comprises the amino acid sequence of SEQ ID NO: 18. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 27. In some embodiments, the GEFI comprises the amino acid sequence of SEQ ID NO: 27. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 28. In some embodiments, the GEFI comprises the amino acid sequence of SEQ ID NO: 28.

Further provided herein are constructs encoding the GEFI described herein.

In some aspects, provided herein is a cell comprising the GEFI described herein.

In some aspects, provided herein are kits comprising the GEFI described herein.

In some aspects, a GEFI, construct, cell, or kit described herein may be used in a method of detecting protease activity, detecting protein-protein interaction, or detecting GPCR agonists.

In another aspect, provided herein are methods of determining whether an agent is a G-protein coupled receptor (GPCR) agonist. In some embodiments, the method comprises providing a system containing a genetically-encoded fluorescent indicator (GEFI), wherein the GEFI comprises a G-protein coupled receptor (GPCR), a circularly-permuted fluorescent protein (cpFP), and an inhibitory molecule. In some embodiments, the C-terminal domain of the GPCR is bound to the cpFP, and the cpFP is bound to the inhibitory molecule. The methods further comprise adding an agent to the system, and detecting the presence or absence of a fluorescent signal after addition of the agent. In some embodiments, the inhibitory molecule inhibits fluorescence from the cpFP in the basal state. In some embodiments, the agent is identified as a GPCR agonist if a fluorescent signal is detected after addition of the agent. The system may comprise a cell.

In some embodiments, the inhibitory molecule is a nanobody. For example, the nanobody may be Nb39. In some embodiments, the inhibitory molecule is bound to the cpFP by a linker. For example, the linker may comprise the sequence LKEDI (SEQ ID NO: 4).

In some embodiments, the GPCR is an opioid receptor. For example, the opioid receptor may be a mu-opioid receptor, a kappa-opioid receptor, or a chimeric opioid receptor.

In some embodiments, the cpFP is bound to the C-terminus of the GPCR by a linker. In some embodiments, the linker comprises FPLKMRMERQGAP (SEQ ID NO: 5). In some embodiments, the cpFP is bound to the protein by the linker GAP.

In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 17. In some embodiments, the GEFI comprises the amino acid sequence of SEQ ID NO: 17. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 21. In some embodiments, the GEFI comprises the amino acid sequence of SEQ ID NO: 21. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 18. In some embodiments, the GEFI comprises the amino acid sequence of SEQ ID NO: 18. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 27. In some embodiments, the GEFI comprises the amino acid sequence of SEQ ID NO: 27. In some embodiments, the GEFI comprises an amino acid sequence having at least 90% sequence identity (e.g. 90%, 91%, 92%, 93%, 94%, 95%, 96%, 97%, 98%, 99%, or 100% sequence identity) with SEQ ID NO: 28. In some embodiments, the GEFI comprises the amino acid sequence of SEQ ID NO: 28.

In some aspects, provided herein is a method of evaluating a protein-protein interaction in a sample. In some embodiments, the method of evaluating a protein-protein interaction in a sample comprises contacting a sample comprising a first protein with a genetically-encoded fluorescent indicator (GEFI). In some embodiments, the GEFI comprises a second protein, a circularly-permuted fluorescent protein (cpFP), and an inhibitory molecule. The cpFP may be any cpFP, including cpGFP or cpRFP as described herein. In some embodiments, the C-terminal domain of first protein is bound to the cpFP, and the cpFP is bound to the inhibitory molecule. The cpFP may be bound to the first protein by a suitable linker, including those described herein. For example, the cpFP may be bound to the first protein by a protein linker described herein. In some embodiments, the method further comprises detecting a fluorescent signal in the sample. In some embodiments, a detectable fluorescent signal in the sample indicates that a protein-protein interaction between the first protein and the second protein has occurred. In some embodiments, the sample comprises a cell. In some embodiments, the first protein is bound to an opioid receptor expressed by the cell. In some embodiments, the method further comprises contacting the sample with an opioid receptor agonist prior to detecting the fluorescent signal in the sample. In some embodiments, when a protein-protein interaction between the first protein and the second protein has occurred, the opioid receptor agonist induces a conformational change in the GEFI such that a fluorescent signal is observed.

In some embodiments, the inhibitory molecule is a nanobody. In some embodiments, the nanobody comprises Nb39. In some embodiments, the inhibitory molecule is bound to the cpFP by a linker. For example, the linker may comprise the sequence LKEDI (SEQ ID NO: 4).

### DESCRIPTION OF THE DRAWINGS

FIG. 1 shows a schematic highlighting an exemplary embodiment of a fluorescent biosensor as described herein. The biosensor comprises a circular-permuted fluorescent protein (cpFP) and an inhibitory molecule bound to the cpFP. In this embodiment, the inhibitory molecule comprises nanobody Nb39. In the basal state, Nb39 is bound to the cpFP, thereby inhibiting fluorophore maturation and preventing a fluorescent signal. Upon conformational change or peptide bond cleavage, Nb39 is removed away from cpFP, allowing the cpFP fluorophore to mature and become fluorescent.

FIG. 2A-2D shows various applications of the biosensor described herein. FIG. 2A shows one application of the biosensor for use in detecting G-protein coupled receptor (GPCR) activation. In FIG. 2A, the GPCR is a Gi-coupled GPCR. The biosensor is inactive in the absence of the Gᵢ-coupled GPCR agonist. Nb39 binds to the GPCR upon agonist activation, subsequently removing itself from circularly permuted green fluorescent protein (cpGFP) and allowing the cpGFP fluorophore to mature. GFP indicates cpGFP signal and FLAG/anti-GFP indicates expression level of the cpGFP. FIG. 2B shows another application of the biosensor for use in detecting Gₛ-coupled GPCR activation. The biosensor is inactive in the absence of agonist. Nb80 binds to the receptor upon agonist activation, removing Nb39 from cpGFP and allowing the cpGFP fluorophore to mature. FIG. 2C shows yet another application of the biosensor for use in detecting protease cleavage events. Protease cleavage releases Nb39 from cpGFP, allowing the cpGFP chromophore to mature. FIG. 2D shows use of the biosensor for detecting protein-protein interaction (PPI). As shown, without PPI, Nb39 inhibits cpGFP fluorophore maturation. PPI brings the protease close to the protease cleavage site, allowing the protease cleavage site to be cleaved. This cleavage removes Nb39 from cpGFP, activating the biosensor.

FIG. 3: Design and characterization of K-SPOTIT1.0. (a) Schematic of SPOTIT. Opioids induce a conformational change in cpGFP, resulting in an increased green fluorescence. OR, opioid receptor. Nb39, G_{αi} protein mimic nanobody 39. cpGFP, circularly permuted GFP from GCaMP6. (b) Testing K-SPOTIT1.0 in HEK293T cells. Two days after infection with lentiviruses expressing K-SPOTIT1.0, cells were stimulated with 10 μM Salvinorin A (Sal A) for 24 hours and then fixed and imaged. (c) K-SPOTIT1.0 fluorophore maturation assay. Two days after infection with lentiviruses expressing K-SPOTIT1.0, cells were stimulated at different time points with 10 μM Sal A for the indicated time periods, and then fixed at the same time point, immunostained, and imaged. GFP, cpGFP fluorescence; FLAG, biosensor protein expression. Scale bars, 20 μm.

FIG. 4. Initial testing of SPOTIT. (a) Confocal imaging of K-SPOTIT1.0 and M-SPOTIT1.0 in fixed HEK293T cells. Two days post infection with lentiviruses expressing SPOTIT, cells were stimulated with 10 μM of Sal A and morphine for 24 hours. Cells were then fixed, immunostained and imaged. (b) Analysis of K-SPOTIT1.0 testing from FIG. 1b. Error bars, standard error of the mean. The mean is represented by the thicker horizontal bar. Stars indicate statistical significance analyzed using an unpaired Student's t-test. Three stars indicate a p-value of 0.0007. “n.s.” indicates no significant difference between the two conditions, p-value of 0.6429. n=5 for each condition. GFP, cpGFP fluorescence; FLAG, protein expression level. Scale bar, 20 μm.

FIG. 5. Characterization of K-SPOTIT1.0's maturation time dependence. Related to FIG. 3c. (a) Two additional fields of view for FIG. 3c. (b) Analysis of the imaging experiment in FIG. 3c. (c) Zoomed in of b. Error bars, standard error of the mean. The mean is represented by each point in the plot. n=13-30 for each time point. GFP, cpGFP fluorescence; FLAG, biosensor protein expression. Scale bar, 20 μm.

FIG. 6. Characterization of the effect of an antagonist on K-SPOTIT1.0 fluorescence post biosensor-maturation. (a) Experimental design for testing the effect of the SPOTIT conformational state on its fluorescence post biosensor maturation. 10 μM Sal A was used to stimulate HEK293T cells expressing K-SPOTIT1.0. After 5 minutes of incubation, the drug was removed, and cells were incubated for 6 hours without drug to allow the cpGFP fluorophore to mature. Then, 10 μM Nor-BNI, a KOR antagonist, was added to the cells and incubated for 1 hour before imaged live. (b) Image analysis of the experiment described in a. Error bars, standard error of the mean. The mean is represented by the thicker horizontal bar. Stars indicate statistical significance analyzed using an unpaired Student's t-test. Four stars indicate a p-value <0.0001. n=38-40 for each condition.

FIG. 7. Mechanistic studies of the role of Nb39 in inhibiting K-SPOTIT1.0 fluorophore maturation in the absence of agonist. (a-c) Schematics of different constructs for SPOTIT mechanistic studies. (d) Confocal imaging of the constructs in a-c in HEK293T cells. Two days after SPOTIT lentiviral infection, cells were stimulated with 10 μM Sal A for 24 hours and then fixed and immunostained for imaging. (e-f) Testing Nb39 in inhibiting cpGFP fluorophore maturation. Two days after cpGFP-Nb39 and cpGFP lentiviral infection, cells were fixed and immunostained for imaging. (g) Analysis of the imaging experiments from a-f. Error bars, standard error of the mean. The mean is represented by the thicker horizontal bar. Stars indicate significance after performing an unpaired Student's t-test. Four stars indicate a p-value <0.0001. Three stars indicate a p-value of 0.001. n=5 for all conditions. GFP, cpGFP fluorescence; Anti-GFP, protein expression. S, Sal A. Scale bars, 20 μm.

FIG. 8. Mechanistic studies of the role of Nb39 in inhibiting K-SPOTIT1.0 fluorophore maturation in the basal state. Related to FIG. 7. Three additional fields of view for the experiments shown and described in FIG. 7. GFP, cpGFP fluorescence; anti-GFP, biosensor expression level. Scale bar, 20 μm.

FIG. 9. Testing the sensitivity of K-SPOTIT1.0 to agonist exposure time. (a) Schematic for characterizing K-SPOTIT1.0 agonist exposure time dependence. Cells were stimulated with 10 μM Sal A for different time periods as indicated in a, and then Sal A was removed and cells were further incubated for a total of 24 hours. (b) Imaging characterization of K-SPOTIT1.0 agonist exposure time dependence in fixed and immunostained HEK293T cells. (c) Analysis of agonist exposure time dependence from the imaging experiment in b. Values above the dots represent the fold-change of GFP signal compared to the no drug condition. Error bars, standard error of the mean. The mean is represented by the thicker horizontal bar. n=5 for all conditions. GFP, cpGFP fluorescence; FLAG, biosensor protein expression. S, Sal A. Scale bar, 20 m.

FIG. 10. Characterization of K-SPOTIT1.0's dependence on agonist exposure time. (a) Related to FIG. 9c. Three representative fields of view for the fixed images used to produce the agonist incubation time plot in FIG. 9c. (b) Testing the importance of the KOR-Nb39 bound state in biosensor activation. K-SPOTIT1.0 was exposed to Sal A for 30 seconds. Sal A was removed and the cells were incubated with or without the KOR antagonist, Nor-BNI, for a total of 24 hours. Values above the dots represent the fold-change of GFP signal compared to the no drug condition. Error bars, standard error of the mean. The mean is represented by the thicker horizontal bar. Stars indicate statistical significance analyzed using an unpaired Student's t-test. Four stars indicate a p-value <0.0001. “n.s” indicates no significant difference between the two conditions, p-value of 0.4583. n=5 for each condition. GFP, cpGFP fluorescence; FLAG, biosensor protein expression. Scale bar, 20 μm.

FIG. 11. Design and characterization of M-SPOTIT1.1. (a) DNA constructs of K-SPOTIT with C-terminal truncations. A signal peptide was used to aid membrane trafficking of the biosensor. (b) Confocal imaging of the K-SPOTIT variants. Cells were simulated with 10 μM Sal A for 24 hours, and then fixed, immunostained, and imaged. (c) DNA constructs of M-SPOTIT1.0 and M-SPOTIT1.1. (d) Live cell imaging of M-SPOTIT1.1 in HEK293T. Cells were simulated with 10 μM Morphine for 24 hours and then imaged live. (e) Characterization of the maturation time dependence of M-SPOTIT1.1. Similar to FIG. 1C, except the cells were imaged live for analysis. 10 μM of each drug was used. n=13-30 for each time point. Error bars, standard error of the mean. The mean is represented by each point in the plot. (f) Characterization of M-SPOTIT1.1 agonist exposure time dependence. M-SPOTIT1.1 was exposed to fentanyl and DAMGO for different time periods to assess the sensitivity of the biosensor. The MOR antagonist, naloxone, was added after 30 s stimulation to test the inhibition of biosensor maturation. 10 μM drug concentration was used for all. Cells were imaged live 24-hours post-stimulation. S/N (without naloxone), the fluorescence with agonist divide by that without agonist; S/N (with naloxone), the fluorescence with agonist and naloxone divided by that without agonist but with naloxone. Error bars, standard error of the mean. The mean is represented by the thicker horizontal bar. n=17-36 for each condition. GFP, cpGFP fluorescence; FLAG, biosensor protein expression. Scale bars, 20 μm.

FIG. 12. Characterization of K-SPOTIT variants. Related to FIGS. 11a and 11b. (a) Two additional fields of view of characterizing K-SPOTIT variants with different linker lengths in fixed cells. (b) Analysis of the imaging experiment in FIG. 11b when cells were imaged live. Error bars, standard error of the mean. The mean is represented by the thicker horizontal bar. Stars indicate statistical significance analyzed using an unpaired Student's t-test. Four stars indicate a p-value <0.0001. n=30 for each condition. GFP, cpGFP fluorescence; FLAG, biosensor protein expression. Scale bar, 20 μm.

FIG. 13. Characterization of M-SPOTIT1.1 maturation time dependence. Related to FIG. 11e. Five representative fields of view for the live-cell images used to produce the maturation time plot. GFP, cpGFP fluorescence; DIC, differential interface contrast. Scale bars, 40 μm.

FIG. 14. Characterization of M-SPOTIT1.1 agonist exposure time dependence. Related to FIG. 11f. Five representative fields of view of the live-cell images used to produce the agonist incubation time plot. GFP, cpGFP fluorescence; DIC, differential interface contrast. Scale bar, 40 μm.

FIG. 15. M-SPOTIT1.1 pH dependence and selectivity characterization. (a) M-SPOTIT1.1 under different imaging conditions. Two days after infection with lentiviruses expressing M-SPOTIT1.1, cells were stimulated with 10 μM fentanyl for 24 hours. Cells were imaged either live or fixed at pH 7 or 9. (b) Analysis of the imaging experiment in a. Error bars, standard error of the mean. The mean is represented by the thicker horizontal bar. Values above the dots represent the fold-change of GFP signal compared to the no drug condition. n=10 for each condition. (c) pH titration of M-SPOTIT1.1 in fixed HEK293T cells. Two days after infection with lentiviruses expressing M-SPOTIT1.1, cells were stimulated with 10 μM fentanyl or beta-endorphin for 24-hours and then fixed and immunostained. The cells were then imaged at the pH indicated. (d) Analysis of the imaging experiment in c. Error bars, standard error of the mean. The mean is represented by each point in the plot. n=9-10 for each data point. (e) Characterization of M-SPOTIT1.1 drug selectivity. Two days after infection with lentiviruses expressing M-SPOTIT1.1, cells were stimulated with 10 μM of each drug for 24 hours, and then fixed, immunostained and imaged at pH 9. Error bars, standard error of the mean. The mean is represented by the thicker horizontal bar. Values above the dots represent the fold-change of GFP signal compared to no drug condition. n=10 for each condition. GFP, cpGFP fluorescence; anti-GFP, biosensor protein expression level. Scale bars, 20 μm.

FIG. 16. Protonated and deprotonated states of the cpGFP fluorophore.

FIG. 17. Confocal imaging characterization of M-SPOTIT1.1 in live and fixed cells. Related to FIG. 15a. Four representative fields of view for characterizing the fluorescence decrease of M-SPOTIT1.1 after formaldehyde fixation. GFP, cpGFP fluorescence; anti-GFP, protein expression level. Scale bar, 20 μm.

FIG. 18. pH titration of M-SPOTIT1.1 in fixed HEK cells. Related to FIG. 15c. Five representative fields of view for pH titration. GFP, cpGFP fluorescence; anti-GFP, protein expression level. Scale bar, 20 μm.

FIG. 19. Characterization of M-SPOTIT1.1's drug selectivity. Related to FIG. 15e. Five representative fields of view of the fixed-cell images used to characterize M-SPOTIT1.1's response to different drugs. GFP, cpGFP fluorescence; anti-GFP, protein expression level. Scale bar, 20 μm.

FIG. 20. Testing M-SPOTIT1.1 in neuron culture. (a) Imaging of M-SPOTIT1.1 in rat cortical neuron culture. Seven days after infection with AAV-viruses expressing TREp-M-SPOTIT1.1-IRES-mCherry and Synapsin-tTA, neurons were stimulated with fentanyl and beta-endorphin for 24-hours. Cells were fixed and imaged at pH 9. GFP, cpGFP fluorescence; mCherry, protein expression level. (b) Analysis of the imaging experiment in a. Error bars, SEM. The mean is represented by the thicker horizontal bar. Values above the dots represent the fold-change of GFP signal compared to the no drug condition. Stars indicate statistical significance analyzed using an unpaired Student's t-test. Four stars indicate a p-value <0.0001. Two stars indicate a p-value of 0.004. n=5 for each condition. (c) Plot of fentanyl titration in cultured neurons. Seven days after infection, M-SPOTIT1.1 infected-neurons were stimulated with the indicated concentration of fentanyl. Neurons were fixed and imaged 24-hours post stimulation. Error bars, standard error of the mean. The mean is represented by each point in the plot. n=5 for each condition.

FIG. 21 Initial testing of M-SPOTIT1.1 in cultured neurons. Related to FIG. 20. Three representative fields of view of the fixed-cell images used to characterize M-SPOTIT1.1's performance in cultured neurons. GFP, cpGFP fluorescence; mCherry, protein expression level. Scale bar, 20 μm.

FIG. 22. Fentanyl titration in cultured neurons. Related to FIG. 20. Three representative views of the fixed-cell images used to characterize M-SPOTIT1.1's performance in cultured neurons. GFP, cpGFP fluorescence; mCherry, protein expression level. Scale bar, 50 μm.

FIG. 23A is a schematic of M-SPOTIT. FIG. 23B shows crystal structure of EGFP (PDB: 2Y0G) and cpGFP (PDB: 3WLC). The critical four amino acids YNSH are highlighted in pink.

FIG. 24A shows testing of M-SPOTIT sensor variants in HEK293T cells. Cells were fixed 24 hours post-stimulation with 10 mM fentanyl or cell culture media alone and imaged at pH 11. GFP, cpGFP signal. Anti-GFP, protein expression level. Scale bar, 20 mm. FIG. 24B shows an analysis plot of FIG. 24A. +F, +fentanyl; —F, −fentanyl. Thick horizontal bars indicate mean and error bars are the standard error of the mean (SEM). Stars indicate significance after a Student's t-test. * p=0.0145 ** p=0.0017, **** p<0.0001 n.s. n=10.

FIG. 25A shows pH titration of the original M-SPOTIT1.1 (left) and M-SPOTIT2 (right). Cells were fixed 24 hours post-simulation and imaged at the indicated pH for one constant field of view. +F, +10 mM fentanyl. —F, with cell culture media. FIG. 25B shows fentanyl titration of M-SPOTIT1.1 and M-SPOTIT2. HEK293T cells were fixed 24 hours post-stimulation and imaged at pH 11. Error bars, SEM. N=14-15. FIG. 25C shows values obtained from the plot in FIG. 25B.

FIG. 26. Screening M-SPOTIT2 against a variety of ligands. HEK293T cells were fixed 24 hours post-stimulation and imaged at pH 11. Ligand concentration=10 mM. Numbers above the points represent SNR. * p=0.0101, **** p o 0.0001. n=9-10.

FIG. 27A shows testing M-SPOTIT2 in rat cortical neuronal culture. Neuronal culture (including neurons and glial cells) was fixed 20 hours after stimulation and imaged at pH 11. GFP, cpGFP fluorescence. DIC, differential interference contrast. Scale bar, 50 mm. FIG. 27B shows an analysis plot of A. +F, +10 mM fentanyl. —F, with cell culture media. **** p<0.0001, **p=0.0018. n=9.

FIG. 28A shows a schematic of Red-SPOTIT design. Without opioid activation, Nb39 inhibits cpRFP maturation. In the presence of opioids, NB39 is recruited to the opioid receptor, releasing cpRFP, allowing the fluorophore to mature. FIG. 28B shows Red-SPOTIT testing in HEK293T cell culture. Cells were fixed 24 hours post-stimulation with 10 μM fentanyl or cell culture media alone and imaged at pH 11. RFP, cpRFP signal. Flag, protein expression level. Scale bar, 20 μm.

FIG. 29A-29B show a time-gated sensor design and testing in HEK 293T cell culture. FIG. 29A shows a schematic of the time-gated sensor design and mechanism. A stimulus such as a small compound or light can be used to induce a protein-protein interaction between A and B, thereby recruiting cpGFP-Nb39 to the membrane. Once recruited, an opioid will recruit Nb39 to OR, releasing cpGFP and allowing the fluorophore to mature. FIG. 29B shows testing of such a time-gated sensor designed for both MOR and KOR. Cells were fixed 24 hours post stimulation with 10 μM fentanyl or Salvinorin A, 1 μM rapamycin, or cell culture media alone and imaged at pH 11. Scale bar, 20 μm.

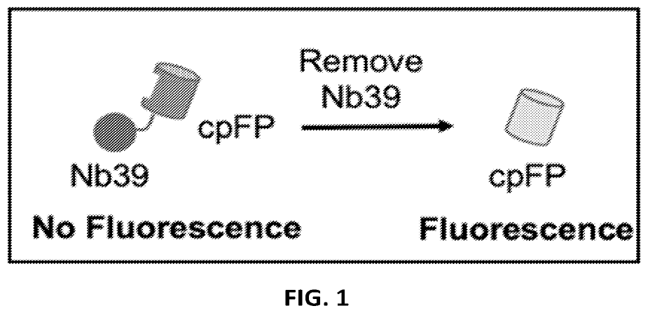

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “basal state” refers to conditions in which no external factors are added to an environment containing a GEFI as described herein. For example, the term basal state refers to conditions in which no external agonists or antagonists for a given receptor are added to the environment containing the GEFI. As another example, the term basal state may refer to conditions in which no binding partners are added to an environment containing the GEFI.

As used herein, the term “circularly permuted” in reference to a fluorescent protein refers to a fluorescent protein in which the N- and C-termini are fused, typically using a peptide linker, while new termini are formed near the chromophore. Such a structure imparts greater mobility to the fluorescent protein than that of the native variant, allowing greater lability of the spectral characteristics.

As used herein, the term “nanobody” refers to an antibody fragment having a single monomeric variable antibody domain. Nanobodies are able to bind selectively to a specific antigen. Nanobodies typically have a molecular weight of only 12-15 kDa, and are thus much smaller than common antibodies, Fab fragments, and single-chain variable fragments.

### DETAILED DESCRIPTION

Provided herein are fluorescent biosensors and uses thereof. In some aspects, provided herein are fluorescent biosensors and methods of use for detecting various signaling events. The fluorescent biosensors described herein are activatable in response to a particular biological event, and are therefore referred to herein as “genetically encoded fluorescent indicators” or “GEFIs”.

The GEFIs, cells, and kits described herein find use in various applications, including detecting GPCR agonists, PPIs, and protease cleavage. The GEFIs for GPCR agonist detection may be used for detection of GPCR agonists in vitro and in vivo. Accordingly, the GEFIs may be used for both drug screening and novel biological discoveries in living systems. In some embodiments, multiple GEFIs as described herein may be used for multiplexed methods, such as evaluation of opioid agonists for multiple opioid receptors at one time, or evaluation of multiple PPIs at one time. The PPI detection and protease detection assays may be useful for biological discoveries and assay and kit development for drug screening.

The genetically encoded fluorescent indicators (GEFIs) described herein are based on fluorescent proteins, and can be used to visualize and quantify cellular events (e.g. enzyme activity, conformational changes of proteins, protein-protein interactions, GPCR agonist binding, etc.) in vivo, including living cells, tissues, or whole organisms. The GEFIs described herein convert such biochemical events into visible, fluorescent signals that can be detected using standard optical equipment.

In some embodiments, provided herein are GEFIs comprising a circularly-permuted fluorescent protein (cpFP), and an inhibitory molecule bound to the cpFP. In general, the inhibitory molecule inhibits fluorescence from the cpFP in the basal state. A change in one or more conditions from the basal state may induce disinhibition of cpFP fluorescence. For example, cpFP fluorescence may be disinhibited upon conformational change of the GEFI and/or disruption of the bond between the cpFP and the inhibitory molecule.

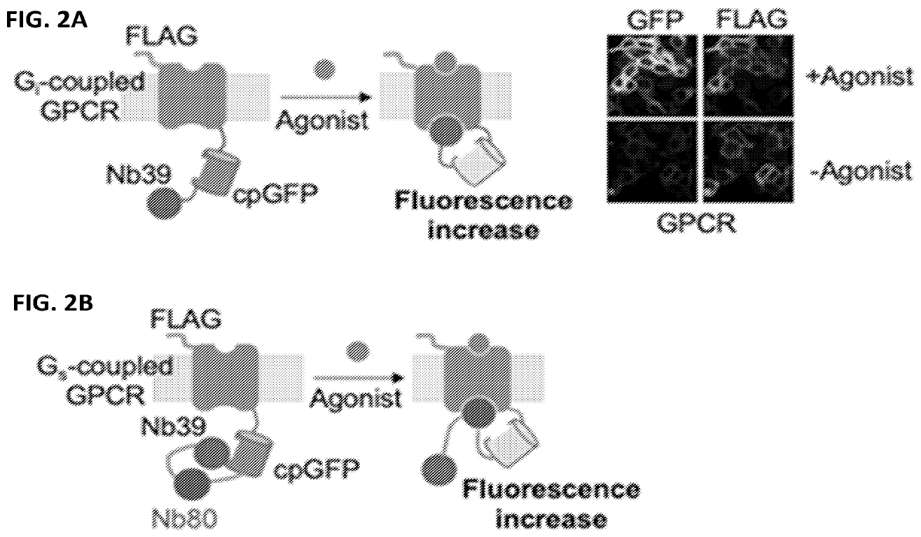

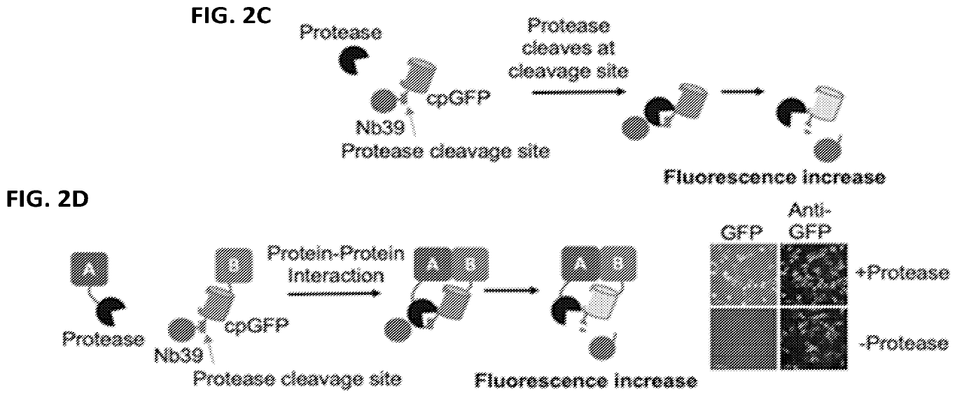

In some embodiments, cpFP fluorescence is disinhibited upon conformational change of the GEFI. A conformational change of the GEFI may include, for example, movement of the inhibitory molecule away from the cpFP. Movement away from the cpFP may occur, for example, due to binding of the inhibitory molecule to a site on a different protein, thereby creating distance between the inhibitory molecule and the cpFP. Such distance may be generated without actual cleavage of the bond between the inhibitory molecule in the cpFP. Such an embodiment is visualized, for example, in FIG. 2A. Such embodiments may be particularly useful for identifying agonists of a receptor of interest, wherein agonist activity permits binding of the inhibitory molecule to a site on the receptor, thereby facilitating distancing of the inhibitory molecule from the cpFP and permitting a fluorescent signal to occur. In some embodiments, cpFP fluorescence is disinhibited upon cleavage of the bond (e.g. linker) connecting the inhibitory molecule and the cpFP. For example, one or more enzymes (e.g. proteases) may cleave the linker, thereby freeing the cpFP from the inhibitory molecule and permitting fluorescence to occur. Such an embodiment is visualized, for example, in FIG. 2C. Such embodiments may be particularly useful for identifying proteases or signifying protease activity in a system. In some embodiments, the GEFI may be connected to a protein (e.g. protein A) and the protease of interest may be connected to another protein (e.g. protein B). Interaction between protein A and protein B brings the protease in proximity to the GEFI, thereby permitting cleavage of the linker connecting the cpFP and the inhibitory molecule. Accordingly, such an embodiment may be useful for studying protein-protein interactions, wherein interaction between the proteins is signified by a fluorescent signal being visible from the cpFP. Such an embodiment is visualized, for example, in FIG. 2D.

Any suitable cpFP may be used, including commercially available circularly-permuted fluorescent proteins or fluorescent proteins that are circularly-permuted in a custom fashion. Exemplary cpFPs, include, for example, circularly-permuted variants of green fluorescent protein (cpGFP), red fluorescent protein (cpRFP) blue fluorescent protein (cpBFP), cyan fluorescent protein (cpCFP), yellow fluorescent protein (cpYFP), violet-excitable green fluorescent protein (cpSapphire), or enhanced green fluorescent protein (cpEGFP). In some embodiments, the cpFP is a cpGFP, comprising the amino acid sequence

(SEQ ID NO: 1)
NVYIKADKQKNGIKANFHIRHNIEDGGVQLAYHYQQNTPIGDGPV
LLPDNHYLSVQSKLSKDPNEKRDHMVLLEFVTAAGITLGMDELYK
GGTGGSMVSKGEELFTGVVPILVELDGDVNGHKFSVSGEGEGDAT
YGKLTLKFICTTGKLPVPWPTLVTTLTYGVQCFSRYPDHMKQHDF
FKSAMPEGYIQERTIFFKDDGNYKTRAEVKFEGDTLVNRIELKGI
DFKEDGNILGHKLEYN

In some embodiments, the cpFP is a cpRFP, comprising the amino acid sequence:

(SEQ ID NO: 26)
RRKWIKAGHAVRAIGRLSSPVVSERMYPEDGVLKSEIKKGLRLKD
GGHYAAEVKTTYKAKKPVQLPGAYIVDIKLDIVSHNEDYTIVEQC
ERAEGRHPTGGRDELYKGGTGGSLVSKGEEDNMAIIKEFMRFKVH
MEGSVNGHEFEIEGEGEGRPYEAFQTAKLKVTKGGPLPFAWDILS
PQFTYGSKAYIKHPADIPDYFKLSFPEGFRWERVMNFEDGGIIHV
NQDSSLQDGVFIYKVKLRGTNFPPDGPVMQKKTMGWEATRDQLTE
EQIAEFKEAFSLFDKDGDGTITTKELGTVLRSLGQNPTEAELQDM
INEVDADGDGTFDFPEFLTMMARRMNDTDSEVEIREAFRVFDNDG
NGYIGAAELRHVMTDLGEKLTDEEVDEMIRVADIDGDGQVNYEEF
VQMMTAK.

Any suitable inhibitory molecule may be used. In some embodiments, the inhibitory molecule is a nanobody. In some embodiments, the inhibitory molecule may be a G-protein mimic. For example, the inhibitory molecule be a G-protein mimic nanobody. In some embodiments, the inhibitory molecule is a G_{αi} mimic nanobody. In particular embodiments, the inhibitory molecule is the G_{αi} protein mimic nanobody Nb39. In some embodiments, the inhibitory molecule is Nb39, comprising the amino acid sequence MAQVQLVESGGGLVRPGGSLRLSCVDSERTSYPMGWFRRAPGKEREFVASITWSGIDPT YADSVADRFTTSRDVANNTLYLQMNSLKHEDTAVYYCAARAPVGQSSSPYDYDYWGQ GTQVTVSS (SEQ ID NO: 2). GEFIs comprising Nb39 may be particularly useful for identifying GPCR agonists. For example, a GEFI comprising Nb39 may be useful for identifying Gᵢ-coupled GPCR agonists. Without wishing to be bound by theory, it is thought that cpFP fluorescence is inhibited by Nb39 by the nanobody preventing maturation of the fluorophore from occurring. When Nb39 is distanced from or cleaved from the cpFP, such as by an agonist binding to the GPCR (e.g. Gᵢ-coupled GPCR) and thereby inducing binding of Nb39 to a site on the GPCR, the fluorophore is allowed to mature, thereby permitting a fluorescent signal. As another example, GEFIs comprising Nb39 and a Gₛ protein mimic, may be used to study Gₛ-coupled GPCR agonists. In some embodiments, the Gs protein mimic is the nanobody Nb80. For example, the Gs protein mimic may be the nanobody Nb80 comprising the amino acid sequence

(SEQ ID NO: 3)
QVQLQESGGGLVQAGGSLRLSCAASGSIFSINTMGWYRQAPGKQR
ELVAAIHSGGSTNYANSVKGRFTISRDNAANTVYLQMNSLKPEDT
AVYYCNVKDYGAVLYEYDYWGQGTQVTVSS

For example, the GEFI may comprise a Gs-coupled GPCR, a cpFP, Nb39, and Nb80. The Nb80 may reside in between the cpfP and Nb39. For example, the GEFI may comprise, from N-terminus to C-terminus, GPCR, cpFP, Nb80, and Nb39. Addition of a Gs-GPCR agonist would activate the GPCR, permitting interaction of the Nb80 with the receptor, thereby distancing Nb39 from the cpFP and permitting fluorescence to occur. Such an embodiment is shown, for example, in FIG. 2B. The Nb80 (or other Gs-mimic nanobody) may be joined to the inhibitory molecule (e.g. Nb39) by a suitable linker. In some embodiments, the linker comprises 1-20 amino acids. In some embodiments, the linker joining Nb80 to Nb39 comprises the amino acid sequence LKE. The Nb80 may be joined to the cpFP by an linker. The linker may be the any suitable linker, including linkers referred to herein as the “inhibitor linker”.

The inhibitory molecule (e.g. Nb39) may be bound to the cpFP by a suitable linker. In some embodiments, the inhibitory molecule is bound to the C-terminal end of the cpFP. The linker connecting the inhibitory molecule to the cpFP is referred to herein as the “inhibitor linker”. In some embodiments, the inhibitor linker comprises 1-20 amino acids. For example, the inhibitor linker may comprise 1, 2, 3, 4, 5, 6, 7, 8, 9, 10, 11, 12, 13, 14, 15, 16, 17, 18, 19, 20 amino acids. In some embodiments, the inhibitor linker comprises the amino acid sequence LKEDI (SEQ ID NO: 4). In some embodiments, the inhibitor linker contains a protease cleavage site. Any suitable protease cleavage site may be used. In some embodiments, the protease cleavage site is a Tobacco etch virus protease cleavage site (TEVcs).

In some embodiments, the GEFI further comprises a protein. The protein may be a natural protein or a synthetic variant. For example, synthetic (e.g. engineered) variants may comprise one or more mutations to facilitate binding of the cpFP to the protein, expression of the construct in a cell, insertion of the protein within a desired location, minimization of undesirable interactions with other components, etc.). In some embodiments, the protein is a G-protein coupled receptor. In some embodiments, the cpFP is bound to the protein. The cpFP may be bound to the C-terminal domain of the GPCR. For example, the cpFP may be bound to the cytosolic c-terminal domain of the GPCR. For example, the GEFI may comprise, from N-terminus to C-terminus, a GPCR, the cpFP, and the inhibitory molecule (e.g. Nb39). When present in a cell, the cpFP and the inhibitory molecule (such as Nb39) would reside in the intracellular component of the cell, thereby permitting detection of various intracellular events.

In some embodiments, the protein (e.g. GPCR) is bound to the cpFP by a linker. A linker joining the protein (e.g. GPCR) to the cpFP is referred to herein as a “protein linker”. In some embodiments, the protein linker comprises 1-20 amino acids. For example, the protein linker may comprise 1, 2, 3, 4, 5, 6, 7, 8, 9, 10, 11, 12, 13, 14, 15, 16, 17, 18, 19, 20 amino acids. In some embodiments, the protein linker comprises the amino acid sequence GAP. In some embodiments, the protein linker comprises the amino acid sequence FPLKMRMERQGAP (SEQ ID NO: 5).

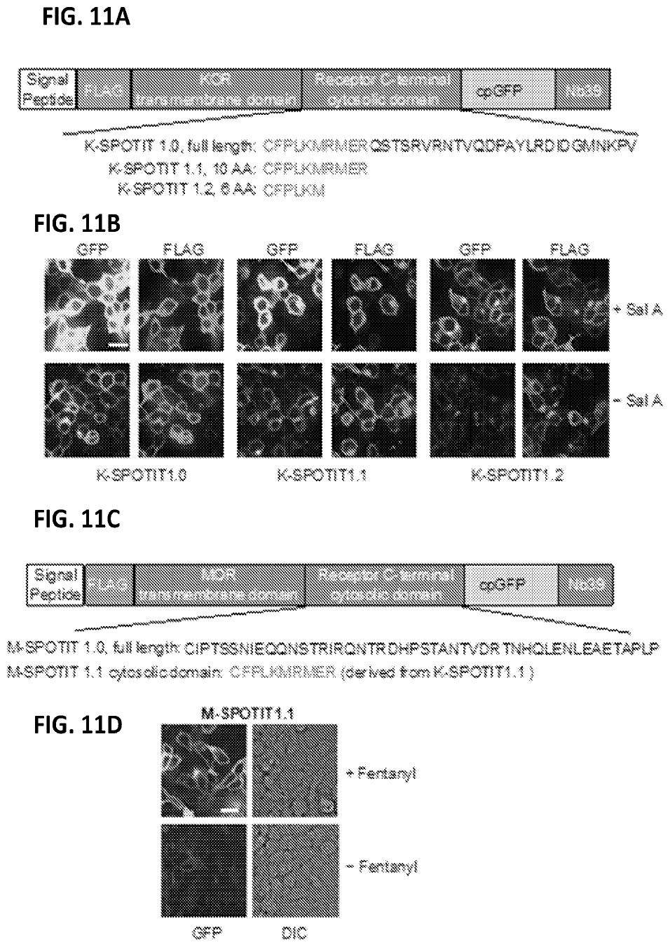

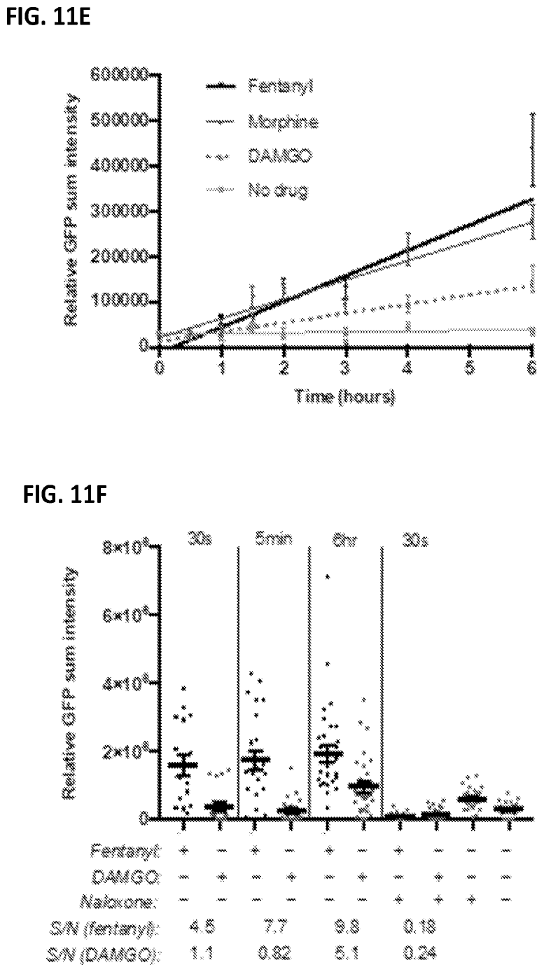

In some embodiments, the protein is a beta-2 adrenergic receptor (B2AR). In some embodiments, the GPCR is an opioid receptor. The opioid receptor may be naturally occurring or may be engineered (e.g. synthetic), as described above. In some embodiments, the C-terminal domain of the opioid receptor may comprise one or more truncation mutations. Suitable c-terminal domain truncations are shown, for example, in FIG. 11A and FIG. 11C. In some embodiments, the opioid receptor may be a mu-opioid receptor (“MOR”), a kappa-opioid receptor (“KOR), a delta-opioid receptor, or a chimeric opioid receptor. A chimeric opioid receptor refers to a synthetic opioid receptor comprising at least one portion from one type of” opioid receptor (e.g. a mu-opioid receptor) and another portion from another type of opioid receptor (e.g. a kappa-opioid receptor). For example, the GEFI described herein as M-SPOTIT1.1 comprises a chimeric opioid receptor. In some embodiments, the cpFP is bound to the protein (e.g. the opioid receptor) by a linker. The linker connecting the cpFP to the protein is referred to herein as the “cpFP linker”. In some embodiments, the cpFP linker comprises 1-20 amino acids. For example, the inhibitor linker may comprise 1, 2, 3, 4, 5, 6, 7, 8, 9, 10, 11, 12, 13, 14, 15, 16, 17, 18, 19, 20 amino acids. In some embodiments, the inhibitor linker comprises the amino acid sequence FPLKMRMERQGAP (SEQ ID NO: 5). In some embodiments, the cpFP is bound directly to the protein, with no cpFP linker.

In some embodiments, the GEFI comprises a GPCR comprising the amino acid sequence:

(SEQ ID NO: 7)
MDSPIQIFRGEPGPTCAPSACLPPNSSAWFPGWAEPDSNGSAGSE
DAQLEPAHISPAIPVIITAVYSVVFVVGLVGNSLVMFVIIRYTKM
KTATNIYIFNLALADALVTTTMPFQSTVYLMNSWPFGDVLCKIVI
SIDYYNMFTSIFTLTMMSVDRYIAVCHPVKALDFRTPLKAKIINI
CIWLLSSSVGISAIVLGGTKVREDVDVIECSLQFPDDDYSWWDLF
MKICVFIFAFVIPVLIIIVCYTLMILRLKSVRLLSGSREKDRNLR
RITRLVLVVVAVFVVCWTPIHIFILVEALGSTSHSTAALSSYYFC
IALGYTNSSLNPILYAFLDENFKRCFRDFCFPLKMRMERQSTSRV
RNTVQDPAYLRDIDGMNKPVQ.

In some embodiments, the GEFI comprises a GPCR comprising the amino acid sequence:

(SEQ ID NO: 8)
MDSSAAPTNASNCTDALAYSSCSPAPSPGSWVNLSHLDGNLSDPC
GPNRTDLGGRDSLCPPTGSPSMITAITIMALYSIVCVVGLFGNFL
VMYVIVRYTKMKTATNIYIFNLALADALATSTLPFQSVNYLMGTW
PFGTTLCKIVISIDYYNMFTSIFTLCTMSVDRYIAVCHPVKALDF
RTPRNAKIINVCNWILSSAIGLPVMFMATTKYRQGSIDCTLTFSH
PTWYWENLLKICVFIFAFIMPVLIITVCYGLMILRLKSVRMLSGS
KEKDRNLRRITRMVLVVVAVFIVCWTPIHIYVIIKALVTIPETTF
QTVSWHFCIALGYTNSCLNPVLYAFLDENFKRCFREFC.

In some embodiments, the GEFI comprises a GPCR comprising the amino acid sequence:

(SEQ ID NO: 9)
MEPAPSAGAELQPPLFANASDAYPSACPSAGANASGPPGARSASS
LALAIAITALYSAVCAVGLLGNVLVMFGIVRYTKMKTATNIYIFN
LALADALATSTLPFQ
SAKYLMETWPFGELLCKAVLSIDYYNMFTSIFTLTMMSVDRYIAV
CHPVKALDFRTPAKAKLINICIWVLASGVGVPIMVMAVTRPRDGA
VVCMLQFPSPSWYWDTVTKICVFLFAFVVPILIITVCYGLMLLRL
RSVRLLSGSKEKDRSLRRITRMVLVVVGAFVVCWAPIHIFVIVWT
LVDIDRRDPLVVAALHLCIALGYANSSLNPVLYAFLDENFKRCFR
QLCRKPCGRPDPSSFSRAREATARERVTACTPSDGPGGGAAA.

In some embodiments the GEFI comprises the amino acid sequence:

(SEQ ID NO: 10)
MGQPGNGSAFLLAPNRSHAPDHDVTQQRDEVWVVGMGIVMSLIVL
AIVFGNVLVITAIAKFERLQTVTNYFITSLACADLVMGLAVVPFG
AAHILMKMWTFGNFWCEFWTSIDVLCVTASIETLCVIAVDRYFAI
TSPFKYQSLLTKNKARVIILMVWIVSGLTSFLPIQMHWYRATHQE
AINCYANETCCDFFTNQAYAIASSIVSFYVPLVIMVFVYSRVFQE
AKRQLQKIDKSEGRFHVQNLSQVEQDGRTGHGLRRSSKFCLKEHK
ALKTLGIIMGTFTLCWLPFFIVNIVHVIQDNLIRKEVYILLNWIG
YVNSGFNPLIYCRSPDFRIAFQELLC.

In some embodiments, the GEFI comprises a GPCR comprising the amino acid sequence:

(SEQ ID NO: 11)
MDSPIQIFRGEPGPTCAPSACLPPNSSAWFPGWAEPDSNGSAGSE
DAQLEPAHISPAIPVIITAVYSVVFVVGLVGNSLVMFVIIRYTKM
KTATNIYIFNLALADALVTTTMPFQSTVYLMNSWPFGDVLCKIVI
SIDYYNMFTSIFTLTMMSVDRYIAVCHPVKALDFRTPLKAKIINI
CIWLLSSSVGISAIVLGGTKVREDVDVIECSLQFPDDDYSWWDLF
MKICVFIFAFVIPVLIIIVCYTLMILRLKSVRLLSGSREKDRNLR
RITRLVLVVVAVFVVCWTPIHIFILVEALGSTSHSTAALSSYYFC
IALGYTNSSLNPILYAFLDENFKRCFRDFCFPLKMRMERQG.

In some embodiments, the GPCR comprises the amino acid sequence:

(SEQ ID NO: 12)
MDSPIQIFRGEPGPTCAPSACLPPNSSAWFPGWAEPDSNGSAGSEDAQLE
PAHISPAIPVIITAVYSVVFVVGLVGNSLVMFVIIRYTKMKTATNIYIFN
LALADALVTTTMPFQSTVYLMNSWPFGDVLCKIVISIDYYNMFTSIFTLT
MMSVDRYIAVCHPVKALDFRTPLKAKIINICIWLLSSSVGISAIVLGGTK
IPVLIIIVCYTLMILRLKSVRLLSGSREKDRNLRRITRLVLVVVAVFVVC
WVREDVDVIECSLQFPDDDYSWWDLFMKICVFIFAFVTPIHIFILVEALG
STSHSTAALSSYYFCIALGYTNSSLNPILYAFLDENFKRCFRDFCLKELK
E.

In some embodiments, the GPCR comprises the amino acid sequence:

(SEQ ID NO: 13)
MDSSAAPTNASNCTDALAYSSCSPAPSPGSWVNLSHLDGNLSDPCGPNRT
DLGGRDSLCPPTGSPSMITAITIMALYSIVCVVGLFGNFLVMYVIVRYTK
MKTATNIYIFNLALADALATSTLPFQSVNYLMGTWPFGTILCKIVISIDY
YNMFTSIFTLCTMSVDRYIAVCHPVKALDFRTPRNAKIINVCNWILSSAI
GLPVMFMATTKYRQGSIDCTLTFSHPTWYWENLLKICVFIFAFIMPVLII
TVCYGLMILRLKSVRMLSGSKEKDRNLRRITRMVLVVVAVFIVCWTPIHI
YVIIKALVTIPETTFQTVSWHFCIALGYTNSCLNPVLYAFLDENFKRCFR
EFCIPTSSNIEQQNSTRIRQNTRDHPSTANTVDRTNHQLENLEAETAPLP.

In some embodiments, the GPCR comprises the amino acid sequence:

(SEQ ID NO: 14)
MEPAPSAGAELQPPLFANASDAYPSACPSAGANASGPPGARSASSLALAI
AITALYSAVCAVGLLGNVLVMFGIVRYTKMKTATNIYIFNLALADALATS
TLPFQSAKYLMETWPFGELLCKAVLSIDYYNMFTSIFTLTMMSVDRYIAV
CHPVKALDFRTPAKAKLINICIWVLASGVGVPIMVMAVTRPRDGAVVCML
QFPSPSWYWDTVTKICVFLFAFVVPILIITVCYGLMLLRLRSVRLLSGSK
EKDRSLRRITRMVLVVVGAFVVCWAPIHIFVIVWTLVDIDRRDPLVVAAL
HLCIALGYANSSLNPVLYAFLDENFKRCFRQLCFPLKMRMERQ.

In some embodiments, the protein comprises an amino acid sequence having at least 80% (e.g. at least 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

In some embodiments, the GEFI further comprises a signal peptide. For example, the GEFI may further comprise a signal peptide directing the GEFI to the intended location within a cell. For example, the signal peptide may direct insertion of a GPCR (e.g. an opioid receptor) within the desired cell membrane location. In some embodiments, the signal peptide comprises the amino acid sequence MKTIIALSYIFCLVFADYKDDDDA (SEQ ID NO: 15). In some embodiments, the signal peptide comprises the amino acid sequence

(SEQ ID NO: 16)
MRPQILLLLALLTLGLADYKDDDDA

In some embodiments, provided herein a construct encoding a GEFI described herein. For example, the construct may encode a GEFI comprising Nb39, a cpFP, and a GPCR. In some embodiments, the construct may encode a GEFI comprising Nb39, a cpFP, and a mu-opioid receptor. In some embodiments, the construct may encode a GEFI comprising Nb39, a cpFP, and a kappa-opioid receptor. In some embodiments, the construct may encode a GEFI comprising Nb30, a CpFP, and a delta-opioid receptor. In some embodiments, the construct may encode a GEFI comprising Nb39, a cpFP, and a chimeric opioid receptor. Any of the above constructs may additionally comprise Nb80, or a different Gₛ-mimic nanobody. The construct may additionally encode one or more linkers as described above (e.g. an inhibitor linker, a cpFP linker, etc.). In some embodiments, the construct further encodes a signal peptide, as described above.

In some embodiments, the GEFI comprises the sequence:

(SEQ ID NO: 17)
MKTIIALSYIFCLVFADYKDDDDAMDSPIQIFRGEPGPTCAPSACLPPNS
SAWFPGWAEPDSNGSAGSEDAQLEPAHISPAIPVIITAVYSVVFVVGLVG
NSLVMFVIIRYTKMKTATNIYIFNLALADALVTTTMPFQSTVYLMNSWPF
GDVLCKIVISIDYYNMFTSIFTLTMMSVDRYIAVCHPVKALDFRTPLKAK
IINICIWLLSSSVGISAIVLGGTKVREDVDVIECSLQFPDDDYSWWDLFM
KICVFIFAFVIPVLIIIVCYTLMILRLKSVRLLSGSREKDRNLRRITRLV
LVVVAVFVVCWTPIHIFILVEALGSTSHSTAALSSYYFCIALGYTNSSLN
PILYAFLDENFKRCFRDFCFPLKMRMERQSTSRVRNTVQDPAYLRDIDGM
NKPVQGAPNVYIKADKQKNGIKANFHIRHNIEDGGVQLAYHYQQNTPIGD
GPVLLPDNHYLSVQSKLSKDPNEKRDHMVLLEFVTAAGITLGMDELYKGG
TGGSMVSKGEELFTGVVPILVELDGDVNGHKFSVSGEGEGDATYGKLTLK
FICTTGKLPVPWPTLVTTLTYGVQCFSRYPDHMKQHDFFKSAMPEGYIQE
RTIFFKDDGNYKTRAEVKFEGDTLVNRIELKGIDFKEDGNILGHKLEYNL
KEDIMAQVQLVESGGGLVRPGGSLRLSCVDSERTSYPMGWFRRAPGKERE
FVASITWSGIDPTYADSVADRFTTSRDVANNTLYLQMNSLKHEDTAVYYC
AARAPVGQSSSPYDYDYWGQGTQVTVSS.
Such a GEFI is also referred to herein as
“K-SPOTIT1.0”.

In some embodiments, the GEFI comprises the sequence:

(SEQ ID NO: 18)
MRPQILLLLALLTLGLADYKDDDDAMDSSAAPTNASNCTDALAYSSCSPA
PSPGSWVNLSHLDGNLSDPCGPNRTDLGGRDSLCPPTGSPSMITAITIMA
LYSIVCVVGLFGNFLVMYVIVRYTKMKTATNIYIFNLALADALATSTLPF
QSVNYLMGTWPFGTTLCKIVISIDYYNMFTSIFTLCTMSVDRYIAVCHPV
KALDFRTPRNAKIINVCNWILSSAIGLPVMFMATTKYRQGSIDCTLTFSH
PTWYWENLLKICVFIFAFIMPVLIITVCYGLMILRLKSVRMLSGSKEKDR
NLRRITRMVLVVVAVFIVCWTPIHIYVIIKALVTIPETTFQTVSWHFCIA
LGYTNSCLNPVLYAFLDENFKRCFREFCFPLKMRMERQGAPNVYIKADKQ
KNGIKANFHIRHNIEDGGVQLAYHYQQNTPIGDGPVLLPDNHYLSVQSKL
SKDPNEKRDHMVLLEFVTAAGITLGMDELYKGGTGGSMVSKGEELFTGVV
PILVELDGDVNGHKFSVSGEGEGDATYGKLTLKFICTTGKLPVPWPTLVT
TLTYGVQCFSRYPDHMKQHDFFKSAMPEGYIQERTIFFKDDGNYKTRAEV
KFEGDTLVNRIELKGIDFKEDGNILGHKLEYNLKEDIMAQVQLVESGGGL
VRPGGSLRLSCVDSERTSYPMGWFRRAPGKEREFVASITWSGIDPTYADS
VADRFTTSRDVANNTLYLQMNSLKHEDTAVYYCAARAPVGQSSSPYDYDY
WGQGTQVTVSS.
Such a GEFI is also referred to herein as
“M-SPOTIT1.1”.

In some embodiments, the GEFI comprises the sequence:

(SEQ ID NO: 19)
MKTIIALSYIFCLVFADYKDDDDAMEPAPSAGAELQPPLFANASDAYPSA
CPSAGANASGPPGARSASSLALAIAITALYSAVCAVGLLGNVLVMFGIVR
YTKMKTATNIYIFNLALADALATSTLPFQSAKYLMETWPFGELLCKAVLS
IDYYNMFTSIFTLTMMSVDRYIAVCHPVKALDFRTPAKAKLINICIWVLA
SGVGVPIMVMAVTRPRDGAVVCMLQFPSPSWYWDTVTKICVFLFAFVVPI
LIITVCYGLMLLRLRSVRLLSGSKEKDRSLRRITRMVLVVVGAFVVCWAP
IHIFVIVWTLVDIDRRDPLVVAALHLCIALGYANSSLNPVLYAFLDENFK
RCFRQLCRKPCGRPDPSSFSRAREATARERVTACTPSDGPGGGAAAGAPN
VYIKADKQKNGIKANFHIRHNIEDGGVQLAYHYQQNTPIGDGPVLLPDNH
YLSVQSKLSKDPNEKRDHMVLLEFVTAAGITLGMDELYKGGTGGSMVSKG
EELFTGVVPILVELDGDVNGHKFSVSGEGEGDATYGKLTLKFICTTGKLP
VPWPTLVTTLTYGVQCFSRYPDHMKQHDFFKSAMPEGYIQERTIFFKDDG
NYKTRAEVKFEGDTLVNRIELKGIDFKEDGNILGHKLEYNLKEDIMAQVQ
LVESGGGLVRPGGSLRLSCVDSERTSYPMGWFRRAPGKEREFVASITWSG
IDPTYADSVADRFTTSRDVANNTLYLQMNSLKHEDTAVYYCAARAPVGQS
SSPYDYDYWGQGTQVTVSS.

In some embodiments, the GEFI comprises the sequence:

(SEQ ID NO: 20)
MKTIIALSYIFCLVFADYKDDDDAMGQPGNGSAFLLAPNRSHAPDHDVTQ
QRDEVWVVGMGIVMSLIVLAIVFGNVLVITAIAKFERLQTVTNYFITSLA
CADLVMGLAVVPFGAAHILMKMWTFGNFWCEFWTSIDVLCVTASIETLCV
IAVDRYFAITSPFKYQSLLTKNKARVIILMVWIVSGLTSFLPIQMHWYRA
THQEAINCYANETCCDFFTNQAYAIASSIVSFYVPLVIMVFVYSRVFQEA
KRQLQKIDKSEGRFHVQNLSQVEQDGRTGHGLRRSSKFCLKEHKALKTLG
IIMGTFTLCWLPFFIVNIVHVIQDNLIRKEVYILLNWIGYVNSGFNPLIY
CRSPDFRIAFQELLCFPLKMRMERQGAPNVYIKADKQKNGIKANFHIRHN
IEDGGVQLAYHYQQNTPIGDGPVLLPDNHYLSVQSKLSKDPNEKRDHMVL
LEFVTAAGITLGMDELYKGGTGGSMVSKGEELFTGVVPILVELDGDVNGH
KFSVSGEGEGDATYGKLTLKFICTTGKLPVPWPTLVTTLTYGVQCFSRYP
DHMKQHDFFKSAMPEGYIQERTIFFKDDGNYKTRAEVKFEGDTLVNRIEL
KGIDFKEDGNILGHKLEYNLKEDIQVQLQESGGGLVQAGGSLRLSCAASG
SIFSINTMGWYRQAPGKQRELVAAIHSGGSTNYANSVKGRFTISRDNAAN
TVYLQMNSLKPEDTAVYYCNVKDYGAVLYEYDYWGQGTQVTVSSLKEMAQ
VQLVESGGGLVRPGGSLRLSCVDSERTSYPMGWFRRAPGKEREFVASITW
SGIDPTYADSVADRFTTSRDVANNTLYLQMNSLKHEDTAVYYCAARAPVG
QSSSPYDYDYWGQGTQVTVSS.

In some embodiments, the GEFI comprises the sequence:

(SEQ ID NO: 21)
MKTIIALSYIFCLVFADYKDDDDAMDSPIQIFRGEPGPTCAPSACLPPNS
SAWFPGWAEPDSNGSAGSEDAQLEPAHISPAIPVIITAVYSVVFVVGLVG
NSLVMFVIIRYTKMKTATNIYIFNLALADALVTTTMPFQSTVYLMNSWPF
GDVLCKIVISIDYYNMFTSIFTLTMMSVDRYIAVCHPVKALDFRTPLKAK
IINICIWLLSSSVGISAIVLGGTKVREDVDVIECSLQFPDDDYSWWDLFM
KICVFIFAFVIPVLIIIVCYTLMILRLKSVRLLSGSREKDRNLRRITRLV
LVVVAVFVVCWTPIHIFILVEALGSTSHSTAALSSYYFCIALGYTNSSLN
PILYAFLDENFKRCFRDFCFPLKMRMERQGAPNVYIKADKQKNGIKANFH
IRHNIEDGGVQLAYHYQQNTPIGDGPVLLPDNHYLSVQSKLSKDPNEKRD
HMVLLEFVTAAGITLGMDELYKGGTGGSMVSKGEELFTGVVPILVELDGD
VNGHKFSVSGEGEGDATYGKLTLKFICTTGKLPVPWPTLVTTLTYGVQCF
SRYPDHMKQHDFFKSAMPEGYIQERTIFFKDDGNYKTRAEVKFEGDTLVN
RIELKGIDFKEDGNILGHKLEYNLKEDIMAQVQLVESGGGLVRPGGSLRL
SCVDSERTSYPMGWFRRAPGKEREFVASITWSGIDPTYADSVADRFTTSR
DVANNTLYLQMNSLKHEDTAVYYCAARAPVGQSSSPYDYDYWGQGTQVTV
SS.
Such a GEFI is also referred to herein as
“K-SPOTIT1.1”.

In some embodiments, the GEFI comprises the sequence:

(SEQ ID NO: 22)
MKTIIALSYIFCLVFADYKDDDDAMDSPIQIFRGEPGPTCAPSACLPPNS
SAWFPGWAEPDSNGSAGSEDAQLEPAHISPAIPVIITAVYSVVFVVGLVG
NSLVMFVIIRYTKMKTATNIYIFNLALADALVTTTMPFQSTVYLMNSWPF
GDVLCKIVISIDYYNMFTSIFTLTMMSVDRYIAVCHPVKALDFRTPLKAK
IINICIWLLSSSVGISAIVLGGTKVREDVDVIECSLQFPDDDYSWWDLFM
KICVFIFAFVIPVLIIIVCYTLMILRLKSVRLLSGSREKDRNLRRITRLV
LVVVAVFVVCWTPIHIFILVEALGSTSHSTAALSSYYFCIALGYTNSSLN
PILYAFLDENFKRCFRDFCLKELKEGAPNVYIKADKQKNGIKANFHIRHN
IEDGGVQLAYHYQQNTPIGDGPVLLPDNHYLSVQSKLSKDPNEKRDHMVL
LEFVTAAGITLGMDELYKGGTGGSMVSKGEELFTGVVPILVELDGDVNGH
KFSVSGEGEGDATYGKLTLKFICTTGKLPVPWPTLVTTLTYGVQCFSRYP
DHMKQHDFFKSAMPEGYIQERTIFFKDDGNYKTRAEVKFEGDTLVNRIEL
KGIDFKEDGNILGHKLEYNLKEDIMAQVQLVESGGGLVRPGGSLRLSCVD
SERTSYPMGWFRRAPGKEREFVASITWSGIDPTYADSVADRFTTSRDVAN
NTLYLQMNSLKHEDTAVYYCAARAPVGQSSSPYDYDYWGQGTQVTVSS.
Such a GEFI is also referred to herein as
“K-SPOTIT1.2”.

In some embodiments, the GEFI comprises the sequence: MKTIIALSYIFCLVFADYKD

(SEQ ID NO: 23)
DDDAMDSSAAPTNASNCTDALAYSSCSPAPSPGSWVNLSHLDGNLSDPCG
PNRTDLGGRDSLCPPTGSPSMITAITIMALYSIVCVVGLFGNFLVMYVIV
RYTKMKTATNIYIFNLALADALATSTLPFQSVNYLMGTWPFGTILCKIVI
SIDYYNMFTSIFTLCTMSVDRYIAVCHPVKALDFRTPRNAKIINVCNWIL
SSAIGLPVMFMATTKYRQGSIDCTLTFSHPTWYWENLLKICVFIFAFIMP
VLIITVCYGLMILRLKSVRMLSGSKEKDRNLRRITRMVLVVVAVFIVCWT
PIHIYVIIKALVTIPETTFQTVSWHFCIALGYTNSCLNPVLYAFLDENFK
RCFREFCIPTSSNIEQQNSTRIRQNTRDHPSTANTVDRTNHQLENLEAET
APLPGAPNVYIKADKQKNGIKANFHIRHNIEDGGVQLAYHYQQNTPIGDG
PVLLPDNHYLSVQSKLSKDPNEKRDHMVLLEFVTAAGITLGMDELYKGGT
GGSMVSKGEELFTGVVPILVELDGDVNGHKFSVSGEGEGDATYGKLTLKF
ICTTGKLPVPWPTLVTTLTYGVQCFSRYPDHMKQHDFFKSAMPEGYIQER
TIFFKDDGNYKTRAEVKFEGDTLVNRIELKGIDFKEDGNILGHKLEYNLK
EDIQVQLVESGGGLVRPGGSLRLSCVDSERTSYPMGWFRRAPGKEREFVA
SITWSGIDPTYADSVADRFTTSRDVANNTLYLQMNSLKHEDTAVYYCAAR
APVGQSSSPYDYDYWGQGTQVTVSSAAA.
Such a GEFI is also referred to herein as
“M-SPOTIT1.0”.

In some embodiments, the GEFI comprises the sequence:

(SEQ ID NO: 24)
MKTIIALSYIFCLVFADYKDDDDAMEPAPSAGAELQPPLFANASDAYPSA
CPSAGANASGPPGARSASSLALAIAITALYSAVCAVGLLGNVLVMFGIVR
YTKMKTATNIYIFNLALADALATSTLPFQSAKYLMETWPFGELLCKAVLS
IDYYNMFTSIFTLTMMSVDRYIAVCHPVKALDFRTPAKAKLINICIWVLA
SGVGVPIMVMAVTRPRDGAVVCMLQFPSPSWYWDTVTKICVFLFAFVVPI
LIITVCYGLMLLRLRSVRLLSGSKEKDRSLRRITRMVLVVVGAFVVCWAP
IHIFVIVWTLVDIDRRDPLVVAALHLCIALGYANSSLNPVLYAFLDENFK
RCFRQLCFPLKMRMERQGAPNVYIKADKQKNGIKANFHIRHNIEDGGVQL
AYHYQQNTPIGDGPVLLPDNHYLSVQSKLSKDPNEKRDHMVLLEFVTAAG
ITLGMDELYKGGTGGSMVSKGEELFTGVVPILVELDGDVNGHKFSVSGEG
EGDATYGKLTLKFICTTGKLPVPWPTLVTTLTYGVQCFSRYPDHMKQHDF
FKSAMPEGYIQERTIFFKDDGNYKTRAEVKFEGDTLVNRIELKGIDFKED
GNILGHKLEYNLKEDIMAQVQLVESGGGLVRPGGSLRLSCVDSERTSYPM
GWFRRAPGKEREFVASITWSGIDPTYADSVADRFTTSRDVANNTLYLQMN
SLKHEDTAVYYCAARAPVGQSSSPYDYDYWGQGTQVTVSS

In some embodiments, the GEFI comprises the sequence:

(SEQ ID NO: 27)
MKTIIALSYIFCLVFADYKDDDDAMDSPIQIFRGEPGPTCAPSACLPPNS
SAWFPGWAEPDSNGSAGSEDAQLEPAHISPAIPVIITAVYSVVFVVGLVG
NSLVMFVIIRYTKMKTATNIYIFNLALADALVTTTMPFQSTVYLMNSWPF
GDVLCKIVISIDYYNMFTSIFTLTMMSVDRYIAVCHPVKALDFRTPLKAK
IINICIWLLSSSVGISAIVLGGTKVREDVDVIECSLQFPDDDYSWWDLFM
KICVFIFAFVIPVLIIIVCYTLMILRLKSVRLLSGSREKDRNLRRITRLV
LVVVAVFVVCWTPIHIFILVEALGSTSHSTAALSSYYFCIALGYTNSSLN
PILYAFLDENFKRCFRDFCFPLKMRMERQSTSRVRNTVQDPAYLRDIDGM
NKPVQASMVDSSRRKWIKAGHAVRAIGRLSSPVVSERMYPEDGVLKSEIK
KGLRLKDGGHYAAEVKTTYKAKKPVQLPGAYIVDIKLDIVSHNEDYTIVE
QCERAEGRHPTGGRDELYKGGTGGSLVSKGEEDNMAIIKEFMRFKVHMEG
SVNGHEFEIEGEGEGRPYEAFQTAKLKVTKGGPLPFAWDILSPQFTYGSK
AYIKHPADIPDYFKLSFPEGFRWERVMNFEDGGIIHVNQDSSLQDGVFIY
KVKLRGTNFPPDGPVMQKKTMGWEATRDQLTEEQIAEFKEAFSLFDKDGD
GTITTKELGTVLRSLGQNPTEAELQDMINEVDADGDGTFDFPEFLTMMAR
RMNDTDSEVEIREAFRVFDNDGNGYIGAAELRHVMTDLGEKLTDEEVDEM
IRVADIDGDGQVNYEEFVQMMTAKDIMAQVQLVESGGGLVRPGGSLRLSC
VDSERTSYPMGWFRRAPGKEREFVASITWSGIDPTYADSVADRFTTSRDV
ANNTLYLQMNSLKHEDTAVYYCAARAPVGQSSSPYDYDYWGQGTQVTVSS.
Such a GEFI is described in Example 3, and is a
red-SPOTIT for KOR.

In some embodiments, the GEFI comprises the sequence

(SEQ ID NO: 28)
MRPQILLLLALLTLGLADYKDDDDAMDSSAAPTNASNCTDALAYSSCSPA
PSPGSWVNLSHLDGNLSDPCGPNRTDLGGRDSLCPPTGSPSMITAITIMA
LYSIVCVVGLFGNFLVMYVIVRYTKMKTATNIYIFNLALADALATSTLPF
QSVNYLMGTWPFGTTLCKIVISIDYYNMFTSIFTLCTMSVDRYIAVCHPV
KALDFRTPRNAKIINVCNWILSSAIGLPVMFMATTKYRQGSIDCTLTFSH
PTWYWENLLKICVFIFAFIMPVLIITVCYGLMILRLKSVRMLSGSKEKDR
NLRRITRMVLVVVAVFIVCWTPIHIYVIIKALVTIPETTFQTVSWHFCIA
LGYTNSCLNPVLYAFLDENFKRCFREFCFPLKMRMERQASMVDSSRRKWI
KAGHAVRAIGRLSSPVVSERMYPEDGVLKSEIKKGLRLKDGGHYAAEVKT
TYKAKKPVQLPGAYIVDIKLDIVSHNEDYTIVEQCERAEGRHPTGGRDEL
YKGGTGGSLVSKGEEDNMAIIKEFMRFKVHMEGSVNGHEFEIEGEGEGRP
YEAFQTAKLKVTKGGPLPFAWDILSPQFTYGSKAYIKHPADIPDYFKLSF
PEGFRWERVMNFEDGGIIHVNQDSSLQDGVFIYKVKLRGTNFPPDGPVMQ
KKTMGWEATRDQLTEEQIAEFKEAFSLFDKDGDGTITTKELGTVLRSLGQ
NPTEAELQDMINEVDADGDGTFDFPEFLTMMARRMNDTDSEVEIREAFRV
FDNDGNGYIGAAELRHVMTDLGEKLTDEEVDEMIRVADIDGDGQVNYEEF
VQMMTAKDIMAQVQLVESGGGLVRPGGSLRLSCVDSERTSYPMGWFRRAP
GKEREFVASITWSGIDPTYADSVADRFTTSRDVANNTLYLQMNSLKHEDT
AVYYCAARAPVGQSSSPYDYDYWGQGTQVTVSS.
Such a GEFI is described in Example 3, and is a
red-SPOTIT for MOR.

In some embodiments, the GEFI comprises an amino acid sequence having at least 80% (e.g. at least 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 27, or SEQ ID NO: 28.

In some embodiments, the GEFI comprises cpGFP. In some embodiments, the cpGFP additionally comprises the peptide sequence YNSH (SEQ ID NO: 6). In some embodiments, the peptide sequence YNSH (SEQ ID NO: 6) is added to the C-terminus of the cpGFP. In some embodiments, the peptide sequence YNSH (SEQ ID NO: 6) is added to the N-terminus of the cpGFP. In some embodiments, the peptide sequence YNSH (SEQ ID NO: 6) is added to the N-terminus of cpGFP, and the resulting fluorophore is brighter than the fluorophore in a GEFI wherein SEQ ID NO: 6 is not added to the N-terminus of the cpGFP. YNSH may be added to the C-terminus of the cpGFP for any of the GEFIs described herein, including GEFIs containing a kappa-opioid receptor, a mu-opioid receptor, or a chimeric opioid receptor.

In some embodiments, the GEFI comprises the sequence:

(SEQ ID NO: 25)
MRPQILLLLALLTLGLADYKDDDDAMDSSAAPTNASNCTDALAYSSCSPA
PSPGSWVNLSHLDGNLSDPCGPNRTDLGGRDSLCPPTGSPSMITAITIMA
LYSIVCVVGLFGNFLVMYVIVRYTKMKTATNIYIFNLALADALATSTLPF
QSVNYLMGTWPFGTTLCKIVISIDYYNMFTSIFTLCTMSVDRYIAVCHPV
KALDFRTPRNAKIINVCNWILSSAIGLPVMFMATTKYRQGSIDCTLTFSH
PTWYWENLLKICVFIFAFIMPVLIITVCYGLMILRLKSVRMLSGSKEKDR
NLRRITRMVLVVVAVFIVCWTPIHIYVIIKALVTIPETTFQTVSWHFCIA
LGYTNSCLNPVLYAFLDENFKRCFREFCFPLKMRMERQGAPYNSHNVYIK
ADKQKNGIKANFHIRHNIEDGGVQLAYHYQQNTPIGDGPVLLPDNHYLSV
QSKLSKDPNEKRDHMVLLEFVTAAGITLGMDELYKGGTGGSMVSKGEELF
TGVVPILVELDGDVNGHKFSVSGEGEGDATYGKLTLKFICTTGKLPVPWP
TLVTTLTYGVQCFSRYPDHMKQHDFFKSAMPEGYIQERTIFFKDDGNYKT
RAEVKFEGDTLVNRIELKGIDFKEDGNILGHKLEYNLKEDIMAQVQLVES
GGGLVRPGGSLRLSCVDSERTSYPMGWFRRAPGKEREFVASITWSGIDPT
YADSVADRFTTSRDVANNTLYLQMNSLKHEDTAVYYCAARAPVGQSSSPY
DYDYWGQGTQVTVSS.
Such a GEFI is also referred to herein as
“M-SPOTIT2”.

In some embodiments, the GEFI comprises an amino acid sequence having at least 80% (e.g. at least 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 25.

In some embodiments, a cell may be transfected with a construct described herein, thereby permitting expression of the GEFI encoded by the construct within the cell. The construct may be delivered to the cell using a suitable delivery vehicle, including viral vectors (e.g. lentiviral vectors, adeno-associated viral vectors, etc.) and non-viral vectors.

In some embodiments, provided herein is a cell comprising a GEFI as described herein. For example, the cell may be transfected with a construct as described herein, and exposed to suitable conditions to permit expression of a GEFI encoded thereby within the cell. For example, the cell may express a GEFI comprising a cpFP and an inhibitory molecule bound thereto. For example, a cell may express a cpFP bound to Nb39. In some embodiments, the cell expresses the cpFP, inhibitory molecule, and a protein (e.g. receptor) of interest. For example, the cell may express a GPCR, a cpFP bound thereto, and an inhibitory molecule (e.g. Nb39) bound to the cpFP. The cell may be any suitable cell, including mammalian cells, bacterial cells, fungal cells, etc.

In some embodiments, provided herein is a kit comprising a GEFI, cell, or construct as described herein. For example, the kit may contain a construct encoding a GEFI comprising a cpFP and an inhibitory molecule bound thereto. The kit may be used, for example, to transfect a cell with the construct, thereby generating a cell expressing the GEFI for use in investigating protein-protein interactions, protease activity, cell-signaling events, GPCR agonists, and the like.

In some embodiments, the kit further comprises transfection reagents. Suitable transfection reagents include, for example, cationic polymers (e.g. PEI, lipofectamine, etc.), calcium phosphate, buffers, etc.

In some aspects, providing herein is a method for identifying a GPCR agonist. In some embodiments, methods for identifying a GPCR agonist comprise providing system containing the GEFI as described herein, and adding one or more agents to the system. In some embodiments, the system is a cell. In some embodiments, the cell is present within a sample. For example, identifying a GPCR agonist may be performed in a cell expressing a GEFI described herein. For example, the method may comprise transfecting a cell with a construct encoding a GEFI as described herein, thereby inducing expression of the GEFI within the cell. In some embodiments, the methods comprise adding a compound to the system (e.g. to the cell, to a sample comprising the cell), and measuring a resulting fluorescent signal.

In particular embodiments, the compound is a suspected opioid receptor agonist. In some embodiments, provided herein is a method for identifying an opioid receptor agonist. The methods may comprise providing a system (e.g. a cell) containing a GEFI, wherein the GEFI contains the opioid receptor (e.g. within the cell membrane), the cpFP bound to the C-terminal domain of the opioid receptor (e.g. such that the cpFP is intracellular), and an inhibitory molecule (e.g. Nb39) bound to the cpFP. One or more agents may be added to the system. If the agent is an opioid receptor agonist, a fluorescent signal should be observed. Without wishing to be bound by theory, it is thought that the addition of an opioid receptor agonist induces a change in the receptor, thereby permitting the Nb39 to bind to the receptor. Binding of Nb39 to the receptor causes physical distance between the Nb39 and the cpFP, thereby permitting maturation of the fluorophore to occur and resulting in an observable fluorescent signal. Accordingly, such a method may be particularly useful in methods for identifying novel opioid receptor agonists and/or determining whether known compounds/drugs are opioid receptor agonists.

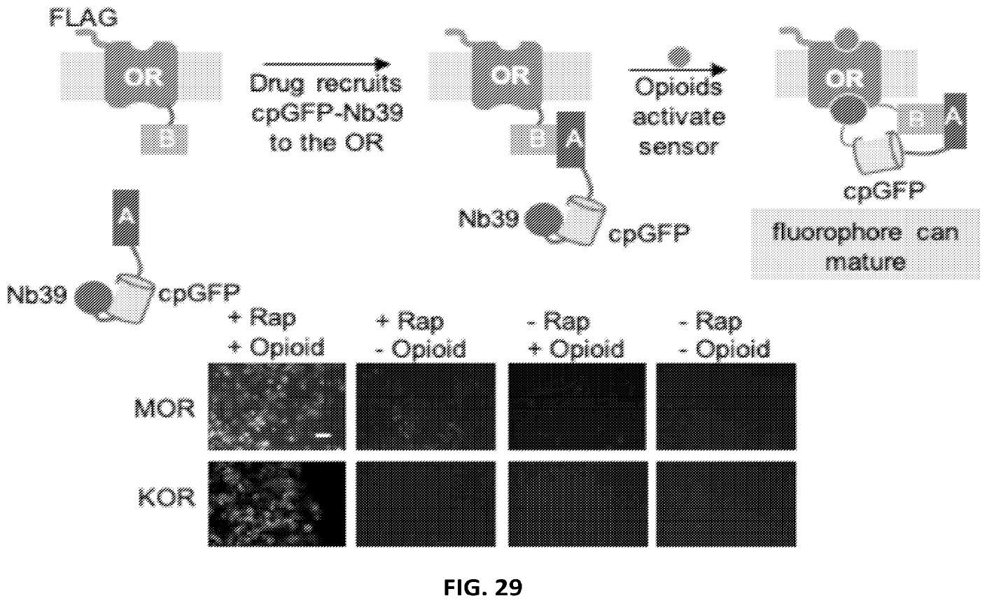

In some embodiments, provided herein is a method for detecting a protein-protein interaction in a sample. The method comprises contacting the sample with a GEFI and visualizing the sample to measure fluorescence and/or determine whether an increase in fluorescence occurs. An observable fluorescence signal or an increase in fluorescence (e.g. relative to a control or baseline measurement) is indicative of a PPI occurring in the sample. In some embodiments, the sample comprises cells. In some embodiments, the sample comprises a first protein suspected of being a binding partner in a protein-protein interaction with a second protein. For example, as shown in FIG. 29 in some embodiments the sample comprises cells expressing a protein “B”. In some embodiments, the protein is bound to a GPCR expressed by the cell. In some embodiments, the protein is bound to an opioid receptor expressed by the cell. In some embodiments, the method comprises contacting the sample with a GEFI as described herein. For example, in some embodiments the method comprises contacting the sample with a GEFI comprising a circularly-permuted fluorescent protein (cpFP), and an inhibitory molecule (e.g. Nb39) bound to the cpFP. In some embodiments, the GEFI further comprises a protein suspected of being a binding partner in a protein-protein interaction with the first protein present within the sample. For example, as shown in FIG. 29A, in some embodiments the GEFI comprises protein “A” attached to the C-terminal end of the cpGFP, such that protein A interacts with protein B. The interaction of protein “A” and protein “B” results in recruitment of the cpGFP-Nb39 GEFI to the cell membrane. An opioid present within or added to the sample will recruit Nb39 to the opioid receptor, thereby distancing Nb39 from the cpGFP and allowing the fluorophore to mature, thus generating a fluorescent signal. In contrast, if a PPI between protein “A” and protein “B” does not occur, Nb39 will remain bound to the cpGFP and no fluorescence will be observed.

In some embodiments, the methods comprise comprising contacting the sample with an opioid receptor agonist prior to detecting the fluorescent signal in the sample. In some embodiments, when a protein-protein interaction between the first protein and the second protein has occurred, the opioid receptor agonist induces a conformational change in the GEFI such that a fluorescent signal is observed. For example, when a protein-protein interaction between the first protein and the second protein has occurred, the GEFI is in close proximity to the opioid receptor expressed by the cell. Accordingly, the addition or presence of an opioid receptor agonist in the sample allows for Nb39 to be recruited to the opioid receptor, thereby inducing a conformational change in the GEFI. Such a conformational change in this instance causes a distancing of the Nb39 away from the cpFP, thereby resulting in fluorophore maturation.

In some embodiments, the method for detecting a PPI in the sample can involve the use of a gating mechanism. For example, a stimulus may be added to the sample in order to induce the PPI, if it does occur in the sample. For example, a stimulus such as light or an added agent can be used in order to induce the PPI in the sample. Upon application of the stimulus, if the PPI is induced and opioids are present in the sample, the inhibitory molecule (e.g. Nb39) would be released from cpGFP as described above. Such a gating mechanism would facilitate investigation of the interaction between two proteins (e.g. protein “A” and protein “B”) under a temporally controlled mechanism. Thus, investigation of the temporal mechanisms of opioid signaling would be possible.

Alternatively, a known PPI interaction can be used to determine whether an agent is an opioid receptor agonist. For example, the interaction between protein “A” and protein “B” may be known to occur. The occurrence of the PPI in the sample would bring the GEFI comprising the first binding partner in a PPI (e.g. protein “A”) into proximity to the cell membrane containing the second binding partner (e.g. protein “B”). A suspected opioid agonist added to or present within the sample would then recruit the inhibitory molecule (e.g. Nb39) to the opioid receptor, thereby releasing the cpFP and generating a measurable fluorescent signal. In contrast, if the agent is not an opioid receptor agonist, Nb39 would not be recruited to the opioid receptor and no fluorescence would be observed.

In some embodiments, the GEFIs described herein may be used in multiplexed methods. For example, multiple GEFIs may be used for multiplexed assessment of multiple potential PPIs. As another example, multiple GEFIs may be used for multiplexed assessment of various potential opioid agonists. For example, different color cpFPs (e.g. red cpFP, green cpFP, etc.) may be used for multiplexed imaging methods. The different color cpFPs may be used in conjunction with different GPCRs present within the GEFI. For example, a first GEFI may contain a first color cpFP and a first GPCR, and a second GEFI may contain a second color cpFP and a second GPCR. For example, a red fluorescent protein-based KOR sensor (e.g. a GEFI comprising cpRFP and a kappa-opioid receptor), and a green fluorescent protein-based MOR sensor (e.g. a GEFI comprising cpGFP and a mu-opioid receptor or a chimeric opioid receptor) can be used to detect agonists for KOR and MOR at the same time. Additionally, such a multiplexed two-color system can be used to perform a high throughput screening for opioid agonists that activate one opioid receptor (e.g. MOR) but not another (e.g. KOR).

For any of the methods described herein, the methods may further comprise obtaining a baseline or control measurement of fluorescence in the system, such as obtaining a baseline measurement of fluorescence in the cell (or in the sample containing the cell) prior to adding a suspected agonist, prior to inducing a PPI, etc. The term “baseline”, “baseline measurement”, and “baseline fluorescence” are used interchangeably to refer to the measurement of fluorescent signal in a system (e.g. in a cell, in a sample comprising a cell) absent the addition of an external stimulus to induce a change in the system. For example, a “baseline measurement” of fluorescence may be measured in the system prior to contacting the system with a stimulus such as a suspected GPCR agonist (e.g. a suspected opioid agonist), light, or any other stimuli that may induce a protein-protein interaction in the sample.

Alternatively, the methods may comprise using multiple samples, each sample containing a cell expressing a GEFI as described herein. One sample may be contacted with a stimulus (e.g. a compound, a light stimulus, etc.) and the other sample (the “control sample”) is contacted with a control agent or no agents at all. For example, one sample may be contacted with a compound (e.g. a suspected GPCR agonist, a suspected opioid receptor agonist) or a stimulus to induce a PPI, and the other sample may be contacted with a control agent or no agents at all. The sample that is not contacted with the compound (e.g. the suspected GPCR agonist) or stimulus is referred to as a “control sample”.

For any of the embodiments described herein, the fluorescence measured in the system after addition of a compound, a stimulus, etc. (e.g. a suspected opioid receptor agonist, a stimulus to induce a PPI in a sample) may be compared to the baseline measurement or control measurement to evaluate whether fluorescence is increased, decreased, or unchanged. An increase in fluorescence indicates that the inhibitory molecule (e.g. Nb39) has been removed from or distanced from the cpFP.

### EXAMPLES

### Example 1

Mu-opioid receptor (MOR) signaling regulates multiple neuronal pathways, including those involved in pain, reward, and respiration. Synthetic opioids have been developed to target MOR for effective pain suppression but also can result in addiction, tolerance, and respiratory suppression¹. It is important to study the site-of-action of opioids to understand their functional effects. Therefore, there is a need to detect the general activation of MOR by opioids in a high-throughput manner and to map where opioids act in the brain at a cellular resolution.

Existing methods to screen for opioids for MOR in cell cultures are limited by either low throughput, low dynamic range, or specificity for β-arrestin2 pathway. These methods have taken advantage of two steps in the opioid signaling cascade: the binding of the opioid to the receptor and the downstream signaling events catalyzed by the receptor activation. Measuring the binding of the opioid to the receptor, through the use of radiolabeled opioids, can be used to infer binding affinities is low-throughput and does not give information about the efficacy of the ligand in activating MOR. Assays that utilize downstream signaling events to screen for opioids include measuring β-arrestin2 recruitment, receptor internalization, G-protein recruitment, cyclic AMP (cAMP) levels, and membrane polarization. The former two kinds of assays rely on the receptor's interaction with β-arrestin2 after receptor activation. Therefore, these two kinds of assays are not optimal for detecting the general activation of the opioid receptor, because there are biased opioid agonists that would preferentially activate the G-protein pathway over the arrestin pathway, resulting in weak arrestin recruitment. G-protein assays involve the incorporation of radiolabeled GTPγS to the activated G-protein but are technically challenging because of radiolabeling and membrane protein extraction. Assays using chimeric G_{αi}/G_{α} q proteins measure the increase of intracellular calcium after opioid activation but require artificial coupling of the chimeric G-proteins with the receptor which is less efficient than endogenous G-protein coupling. cAMP assays, such as those using a transcriptional biosensor, have a poor dynamic range because opioid receptor-induced cAMP inhibition rarely exceeds 60% of the basal state. Finally, membrane polarization caused by opioid receptor activation can be measured either with recording electrodes or fluorescent membrane potential dyes. Electrical recordings are manually challenging and cannot be used for high-throughput selection. Fluorescent membrane dyes that change intensity due to membrane polarization can be used as an indicator for opioid receptor activation, but these dyes only have approximately a 35-50% decrease of membrane fluorescence.

Current methods for detecting opioids and opioid peptides in the animal brain are limited by spatial resolution. State-of-the-art methods using microdialysis coupled to nanoflow liquid chromatography-mass spectrometry (nLC-MS) enable detection of multiple neuropeptides and other neurotransmitters concurrently. However, microdialysis nLC-MS methods have a poor spatial resolution, limited by the probe size on the order of 500 μm. Fast scan cyclic voltammetry (FSVC) has improved spatial resolution due to its smaller probe size of ˜5 μm in diameter, but FSVC has only been used to detect met-enkephalin and not other opioids.

Due to the limitations of existing methods for detecting opioids for MOR, either low-throughput, small dynamic range, low-spatial resolution, or bias towards the β-arrestin2 pathway, there is a need for an assay that allows high-throughput detection of the activation of MOR at a high spatial resolution. To address this need, a genetically-encoded fluorescent biosensor was developed. The biosensor in this example, referred to as “Single-chain Protein-based Opioid Transmission Indicator Tool for MOR” (“M-SPOTIT”), was designed and characterized.

The results described herein demonstrate that M-SPOTIT represents a new and unique mechanism for fluorescent biosensor design and can detect MOR activation, leaving a persistent green fluorescence mark for image analysis. M-SPOTIT shows a signal-to-noise ratio (S/N) up to 9.8-fold and is able to detect as fast as 30-seconds of opioid exposure in cell culture. Additionally, it shows a S/N up to 4.2-fold in neuronal culture and can detect fentanyl with an EC₅₀ of 15 nM. M-SPOTIT will be useful for high-throughput detection of opioids in cell cultures and potentially a cellular-resolution detection of opioids in vivo. M-SPOTIT's novel mechanism can be used as a platform to design other G-protein coupled receptor-based biosensors.

### Materials and Methods

### Plasmids and Cloning

Constructs for HEK293T cell expression were cloned in an ampicillin-resistant lentiviral vector using a cytomegalovirus (CMV) promoter. cpGFP was amplified from AAV-hSyn1-GCaMP6s-P2A-n1s-dTomato (addgene plasmid #51084, Jonathan Ting laboratory.) MOR and KOR sequences were gifts from Bryan Roth (Addgene plasmid #66464 and #66462). Nb39 and Nb80 were synthesized as a gene block from IDT. Standard cloning procedures, such as Q5 polymerase PCR amplification, NEB restriction enzyme digest, and T4 ligation or Gibson assembly were used. Ligated plasmids were transformed into XL1-blue competent cells using heat shock transformation. After full sequencing of all constructs, a point mutation in MSPOTIT1.0 and MSPOTIT1.1 sequences was identified, leading to a isoleucine to threonine mutation at MOR amino acid position 140. This is in the extracellular portion of the third loop and, therefore, does not affect the receptor functionality which depends on an intracellular conformational change. As a result, this construct was used for further experiments.

### HEK293T Cell Culture

HEK293T cells were cultured in complete growth media: 1:1 DMEM (Dublecco's Modified Eagle medium, GIBCO): MEM (Modified Eagle medium, GIBCO), 10% FBS (Fetal Bovine Serum, Sigma), 1% (v/v) penicillin (Gibco), and 1% penstrap (Gibco). 24-well glass bottom plates (Corning) were pre-treated with 200 μL 20 μg/mL human fibronectin (Milipore Sigma) for 10 min at 37° C. under 5% CO₂. Cells were plated at a density so that they would reach 90% confluence on the day of stimulation, for transfection this was next day and for viral infection this was in two days.

HEK Cell Transfection with PEI MAX

PEI MAX (polyethyleneimine, Polysciences) was used for all transfection experiments. 1-3 h after plating, cells were transfected with a homemade PEI max solution. For transfection per well of a 24-well plate, 200 ng SPOTIT DNA and 2 μL PEI max solution (1 mg/mL in H₂O) in 20 μL DMEM was incubated for 10 min at room temperature (RT). After 10 min, 200 μL of complete media was added, and 220 μL of this solution was gently pipetted on top of the plated cells. Cells were incubated at 37° C. with 5% CO₂ until stimulation 24 h later.

### Lentivirus Production

90% confluent HEK293T cells in a T25 flask were transfected with 2.5 μg biosensor DNA, 0.25 μg pVSVG, and 2.25 μg Δ8.9 lentiviral helper plasmid mixed in 200 μL of DMEM without FBS. After 10 min of incubation at room temperature, the DMEM, PEI, and DNA solution was pipetted gently on top of the HEK293T cells in the T25 flask. After two days of incubation at 37° C. with 5% C02, the virus-containing T25 supernatant was collected, aliquoted into 500 μL volumes, flash-frozen using liquid nitrogen, and stored in −80° C. for future use.

### HEK293T Cell Infection

HEK293T cells (less than 20 passages) were plated in 24-well glass bottom plates (Cellvis) pretreated with 350 μL 20 μg/mL human fibronectin (Millipore Sigma) for 10 min at 37° C. HEK293T cells were plated at 40%-60% confluence. For infection of a single well in a 24-well plate, 25-100 μL of each supernatant virus was added gently to the top of the media and incubated for 48 h before stimulation

### HEK Cell Stimulation

20-24 h after transfection and 40-48 h after infection, HEK293T cells were stimulated. Drugs were diluted in pre-warmed complete media to the desired concentration. 100 μL of the diluted drug was gently dropped on top of the plated cells. For stimulation with beta-endorphin and leu-enkephalin, 2 μL of a protease inhibitor cocktail (Millipore Sigma) was added to the 24-well plates prior to peptide stimulation. After the desired drug incubation time, all the media in the well was removed and the well was washed three times with complete media. 400 μL of complete media was added back to the well, and the cells were kept at 37° C. with 5% CO₂ prior to imaging. SPOTIT was found to be sensitive to temperature. Incubation at temperatures lower than 37° C. will increase the background fluorescence. Presumably the cpGFP can be better folded at lower temperate to allow the fluorophore to mature. For all experiments other than chromophore maturation experiments, the cells were imaged 24 h after stimulation.

### HEK Cell Fixation and Immunostaining

For fixation, media was removed from HEK293T cells and 200 μL of fixative (4% formaldehyde in PBS) was added to the wells. After 15 min of fixative incubation, the fixative was removed, and the wells were washed three times with PBS. To permeabilize the cells, 200 μL of cold methanol (˜20° C.) was added to each well. The cells were incubated in −20° C. for 5 min then washed 3× with PBS. For immunostaining, mouse anti-flag (Sigma, F3165) or chicken anti-EGFP (abcam, ab13970) was diluted in a 1% BSA in PBS (phosphate buffered saline) solution to a concentration of 1:1000 antibody:solution. 200 μL of the antibody solution was added to each well, and the cells were incubated with the primary antibody for 30 minutes at RT on a rocker. Then, the cells were washed 3× with PBS and the same volume and concentration of anti-mouse 647 (Life Technologies, A21235) or anti-chicken 647 (abcam, A21449) was added to each well. Again, the cells rocked for 30 min at RT with 5% CO₂ and were washed 3× with PBS. 200 μL of PBS or pH 9 CAPS buffer were added back to the cells, and the cells were imaged.

### Confocal Microscopy of HEK Cells

Confocal imaging was performed on a Nikon inverted confocal microscope with 20× air objective and 60× oil immersion objective, outfitted with a Yokogawa CSU-X1 5000RPM spinning disk confocal head, and Ti2-ND-P perfect focus system 4, a compact 4-line laser source: 405 nm (100 mW) 488 nm (100 mW), 561 nm (100 mW) and 640-nm (75 mW) lasers. The following combinations of laser excitation and emission filters were used for various fluorophores: EGFP/Alexa Fluor 488 (488 nm excitation; 525/36 emission), mCherry (568 nm excitation; 605/52 emission), Alexa Fluor 647 (647 nm excitation; 705/72 emission), and differential interference contrast (DIC). Acquisition times ranged from 1 to 2 s and 50% laser intensity was used for all excitation filters. ORCA-Flash 4.0 LT+sCMOS camera. 1-1.5× magnification was used for the 20× objective and 1× magnification was used for 60×. All images were collected using Nikon NIS-Elements hardware control and processed using NIS-Elements General Analysis 3 software.

### Analysis of HEK293T Cell Images

For live cell 30× magnification images, 10-15 fields of view per well were taken and three technical replicates were performed. For 60× and 20× magnification fixed images, 5-10 images were taken per well. NIS-Elements General Analysis 3 software was used to analyze the images. Data for both the mean FITC (or Cy5) intensity and object areas were taken for all fields of view and all technical replicates. These values were multiplied together to collect the sum FITC (or Cy5) intensity per field of view. This value was subtracted by the sum FITC (or Cy5) intensity of an empty well to adjust for the background fluorescence. The mean and standard error of the mean (SEM) were calculated for each condition. Two-sided Student's t-tests were used to evaluate the significance between data points. Plots were made using Prism GraphPad software. Fields of view with no cells (due to low confluence or cells lifting during washing steps) were omitted from analysis. These fields of view were identified by a “0” object area.

### K-SPOTIT and M-SPOTIT Initial Testing

HEK293T cells were cultured in 24-well imaging plates and transfected with K-SPOTIT1.0 and M-SPOTIT1.0 lentiviruses following the above protocol. 20-24 h post transfection, the cells were stimulated with 10 μM Salvinorin A for K-SPOTIT and 10 μM morphine for M-SPOTIT. The cells were incubated with drug for 24 h before fixation, FLAG immunostaining, imaging, and data analysis following the previously stated protocols.

### K-SPOTIT Addition of Antagonist

To test the persistence of the SPOTIT signal, HEK293T cells were cultured in 24-well imaging plates and infected with K-SPOTIT1.0 lentivirus following the above protocol. 40-48 h after infection, cells were stimulated for 5 min with 10 μM of Salvinorin A. After 5 min, the cells were washed 3× with complete media and allowed to incubate for 6 h at 37° C. without agonist. After 6 h, 10 μM Nor-BNI was added and incubated for 1 h. Then, the cells were fixed and immunostained for FLAG tag expression. The cells were imaged and analyzed using the protocol stated above.

### K-SPOTIT and M-SPOTIT Linker Optimization

HEK293T cells were cultured in 24-well imaging plates and transfected with SPOTIT DNA coding for the different linker lengths and types following the above protocol. 20-24 h post transfection, the cells were stimulated with 10 μM Salvinorin A for K-SPOTIT and 10 μM morphine for M-SPOTIT. The cells were incubated with drug for 24 h before fixation, FLAG immunostaining, imaging, and data analysis following the previously stated protocols.

### K-SPOTIT and M-SPOTIT Maturation Assays

HEK293T cells were cultured in 24-well imaging plates and infected with K-SPOTIT1.0 and M-SPOTIT1.1 lentiviruses following the above protocol. Wells were plated and infected with K-SPOTIT1.0 lentivirus for the following conditions: 0 h, 0.5 h, 1 h, 1.5 h, 2 h, 3 h, 4 h, 6 h, and 24 h. 0.5 h means the cells were imaged 30 min post stimulation; 1 h means the cells were imaged 1 h post stimulation, and so on. The same conditions were plated for M-SPOTIT1.1, except for 24 h. 40-48 h post infection, the cells were stimulated at different time points with 10 μM Salvinorin A for K-SPOTIT and 10 μM of morphine, fentanyl, and DAMGO for M-SPOTIT1. 1. K-SPOTIT cells were fixed and immunostained with 1:1000 mouse anti-flag antibody and 1:1000 anti-mouse 647 antibody. M-SPOTIT1.1 was imaged live. All wells were imaged at the same time following the imaging and data analysis technique stated above.

K-SPOTIT and M-SPOTIT agonist incubation time assays

HEK293T cells were cultured in 24-well imaging plates and infected with K-SPOTIT1.0 and M-SPOTIT1.1 lentiviruses following the above protocol. Wells were plated and infected with K-SPOTIT1.0 lentivirus for the following conditions: No agonist, 30 s agonist, 5 min agonist, 6 h agonist, 24-36 h agonist, and 30 s agonist followed by antagonist. K-SPOTIT1.0 was stimulated with Salvinorin A for the time points indicated, and M-SPOTIT1.1 was stimulated with both fentanyl and DAMGO for the time points indicated. For example, for the K-SPOTIT1.0 30 s agonist time point, 10 μM Salvinorin A was added to the well for 30 s. After 30 s, the well was washed 3× with complete HEK cell media. 400 μL of fresh complete media was then added back to the well. The same procedure was followed for the other time points of 5 min, 6 h, and 24-26 h. The same procedure was also followed for M-SPOTIT1.1 with fentanyl and DAMGO. For the time points with antagonist added, post-stimulation with agonist, the agonist was washed out 3× and 10 μM of Nor-BNI for K-SPOTIT1.0 and naloxone for M-SPOTIT1.1 was added into the well. This was not washed out. 24-26 h post stimulation, the cells were imaged. K-SPOTIT1.0 was imaged fixed and immunostained for FLAG expression and M-SPOTIT1.1 was imaged live cell.

### Mechanism Testing

HEK293T cells were cultured in 24-well imaging plates and infected with lentivirus DNA constructs used to interrogate the mechanism of SPOTIT (as seen in FIG. 2). 40-48 h post transfection, the cells infected with constructs containing K-SPOTIT were stimulated with 10 μM Salvinorin A. The cells were incubated with Salvinorin A for 24 h before fixation, EGFP immunostaining, and imaging following the protocols described above. For all mechanism studies, GFP and cy5 intensities and object areas were collected as described before. Final figures were made by normalizing the GFP intensity to the protein expression level by dividing the GFP sum intensity values with the cy5 sum intensity values.

### M-SPOTIT Initial pH Testing

HEK293T cells were cultured in 24-well imaging plates and infected with M-SPOTIT1.1 lentiviruses following the above protocol. The following conditions were plated: live cell, fixed pH 7, and fixed pH 9. Live cell conditions were plated on a separate plate from the fixed conditions. 40-48 h post infection, cells were stimulated with fentanyl. 24 h post fentanyl stimulation, live-cell images were taken and the fixed conditions were fixed and immunostained with anti EGFP chicken antibody and anti-chicken 647. After immunostaining, a pH 7 PBS solution or pH 9 CAPS buffer was added to the cells. The fixed cells were then imaged and analyzed using the protocol stated above.

### M-SPOTIT pH Titration

HEK293T cells were cultured in 24-well imaging plates and infected with M-SPOTIT1.1 lentiviruses following the above protocol. The following conditions were plated for fentanyl, beta-endorphin, and no drug stimulations: pH 6, pH 7, pH 8, pH 9, and pH 10. 40-48 h post infection, cells were stimulated with 10 μM fentanyl or 10 μM beta endorphin. 24 h post stimulation, all cells were fixed and immunostained with anti EGFP chicken antibody and anti-chicken 647. After immunostaining, solutions with different pHs were added to the appropriate wells. The following buffers were used pH 6: 1×PBS, pH 7: 1×PBS, pH 8: 100 mM Tris-HCl, pH 9: 100 mM Tris-HCl, pH 10: 100 mM Tris-HCl, pH 11: 100 mM CAPS buffer. All buffers were adjusted with 1 M NaOH and HCl to achieve the correct pH. The cells were then imaged and analyzed using the protocol stated above.

### M-SPOTIT Selectivity

HEK293T cells were cultured in 24-well imaging plates and infected with M-SPOTIT1.1 lentiviruses following the above protocol. 40-48 h post infection, cells were stimulated with 10 μM of different agonists. 24 h post stimulation, cells were fixed and immunostained with anti EGFP chicken antibody and anti-chicken 647. After immunostaining, a pH 9 CAPS buffer was added to the cells. The fixed cells were then imaged and analyzed using the protocol described above.

### AAV Supernatant Production for Neuronal Infection

AAV virus supernatant was used for neuronal culture experiments. 6-well plates were pretreated with human fibronectin for 10 min at 37° C. HEK293T cells were plated on the fibronectin-treated plates, so they were 60-90% confluent. For each well, 0.35 μg AAV expression DNA, 0.29 μg AAV1 serotype, 0.29 μg AAV2 serotype plasmid, and 0.7 μg helper plasmid pDF6 with 80 μL serum-free DMEM and 10 μL PEI max were mixed and incubated for 10 min at room temperature, and then 2 mL complete growth media was added and mixed. The DNA mix was added gently on the top of the cells. HEK293T cells were incubated for 40-48 h at 37° C. and then the virus supernatant was collected. The virus supernatant was stored in sterile Eppendorf tubes (0.5 mL/tube), flash frozen by liquid nitrogen and stored at −80° C.

### Neuronal Culture Experiments.

Frozen rat cortical neurons (Thermo Fisher Scientific, Cat #A1084001) were plated according to the user protocol. The half area 96-well glass plates (Corning, CLS4580-10EA) were coated with 50 μl poly-D-lysine (Gibco, 0.1 mg/ml in water) for 1 h, and then washed twice with ultrapure water. The frozen rat cortical neurons were quickly removed from liquid nitrogen and thawed in the 37° C. water bath by swirling until a small piece of ice was present. The cells were gently transferred to a 50 ml conical tube. To the cells, 1 ml pre-warmed 3:1 ratio of complete neurobasal media (NM) and glial enriching medium (GEM) mix was very slowly dropped in at one drop per second with gentle swirling. NM is composed of neurobasal (Thermo Fisher Scientific) supplemented 2% B27 (Thermo Fisher Scientific), 50 mM HEPES (Thermo Fisher Scientific), 1% Penicillin-Streptomycin (50 units/mL penicillin and 50 μg/mL streptomycin, Thermo Fisher Scientific), and 1% GlutaMAX (Thermo Fisher Scientific). GEM is composed of DMEM (Gibco) supplemented with 10% FBS (Fetal Bovine Serum, Sigma), 2% B27 (Thermo Fisher Scientific), 50 mM HEPES (Thermo Fisher Scientific), 1% Penicillin-Streptomycin (50 units/mL penicillin and 50 μg/mL streptomycin, Thermo Fisher Scientific), and 1% GlutaMAX (Thermo Fisher Scientific). Additional 4 ml of NM:GM (3:1) mix media was added to the cells. Viable cell density was determined by mixing 10 μl of the cell suspension with 10 μl 0.4% Trypan blue and cell counting was performed using hemocytometer. Around 0.25×10⁵ viable cells were plated on each well, and cells were incubated at 37° C. with 5% CO². Half of the media was replaced with fresh 3:1 NM:GEM mix media within 4-24 h after plating.

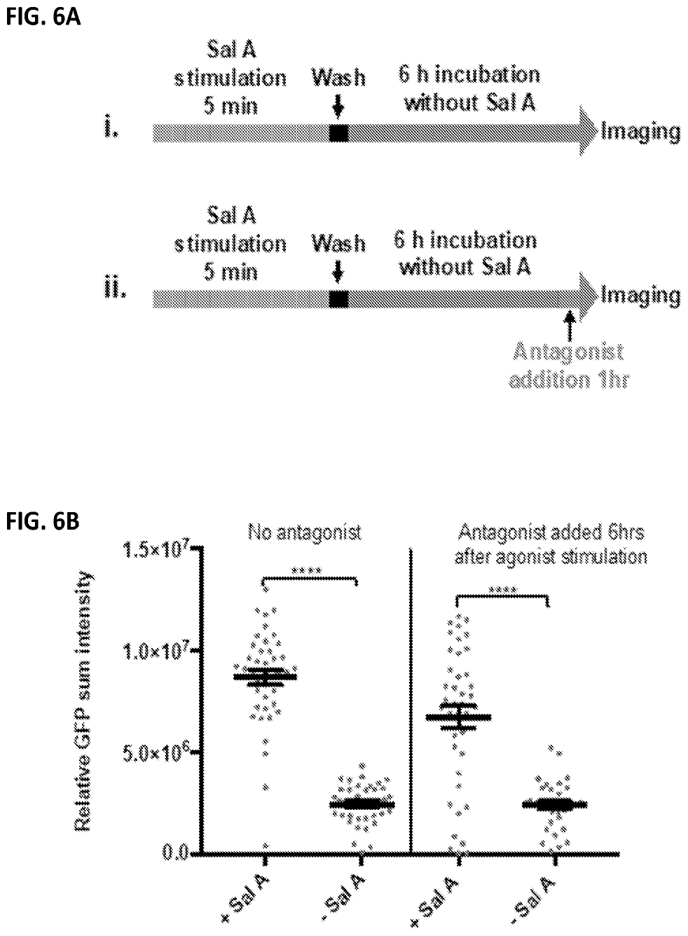

For neuronal infection, supernatant AAV virus mix encoding TREp-M-SPOTIT1.1-IRES-mCherry and Synapsin-tTA (20 μl of each virus) were added the neurons at DIV4-DIV8 (days in vitro). Seven days after infection, neurons were treated with fentanyl and endorphin at the concentrations indicated in FIG. 6 for 24 h and then fixed on ice and imaged at pH 9.

### Analysis of Neuron Images

For initial neuron testing, 5 fields of view of 60× magnification images were taken. For the titration curve, 5 fields of view of 20× magnification images were taken. This experiment was performed twice with similar results. NIS-Elements General Analysis 3 software was used to analyze the images. Data for the mean FITC intensity of the entire image was taken for each field of view. This value was subtracted by the mean FITC intensity of an empty well to adjust for the background of the laser. The mean and standard error of the mean (SEM) were calculated for each condition. Two-sided Student's t-tests were used to evaluate the significance between data points. Plots were made using Prism GraphPad software.

### Results

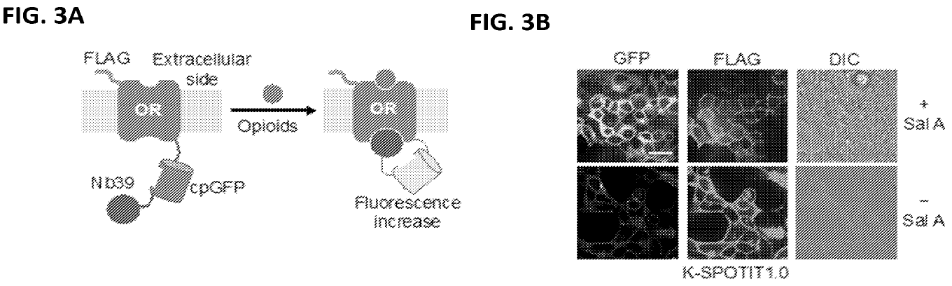

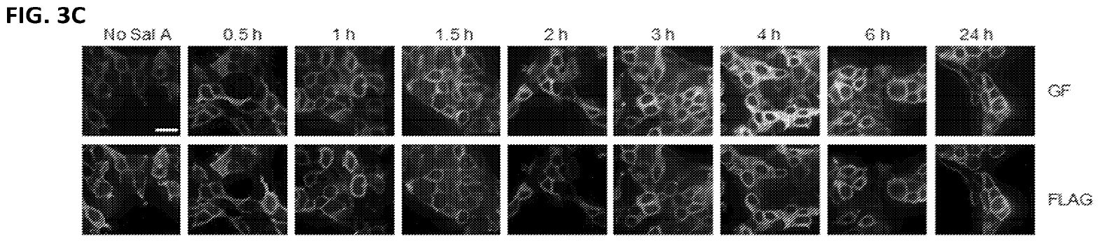

Design and mechanistic understanding of SPOTIT. In the general design of SPOTIT, the circularly-permuted green fluorescent protein (cpGFP) described in Nagai, T., Sawano, A., Park, E. S. & Miyawaki, A. Circularly permuted green fluorescent proteins engineered to sense Ca2+. Proc. Natl. Acad. Sci. 98, 3197-3202 (2001), the entire contents of which are incorproated herein by reference for all purposes, was inserted between the C-terminus of an opioid receptor and a G_{αi}-mimic nanobody, Nb39, described in Huang, W. et al. Structural insights into p-opioid receptor activation. Nature 524, 315-321 (2015), the entire contents of which are incorporated herein by reference for all purposes (FIG. 3a). The biosensor was designed as shown in FIG. 3a so that cpGFP would adopt an open-conformation without opioid agonist, and change to a closed-conformation when Nb39 interacts with the activated receptor in the presence of agonist.

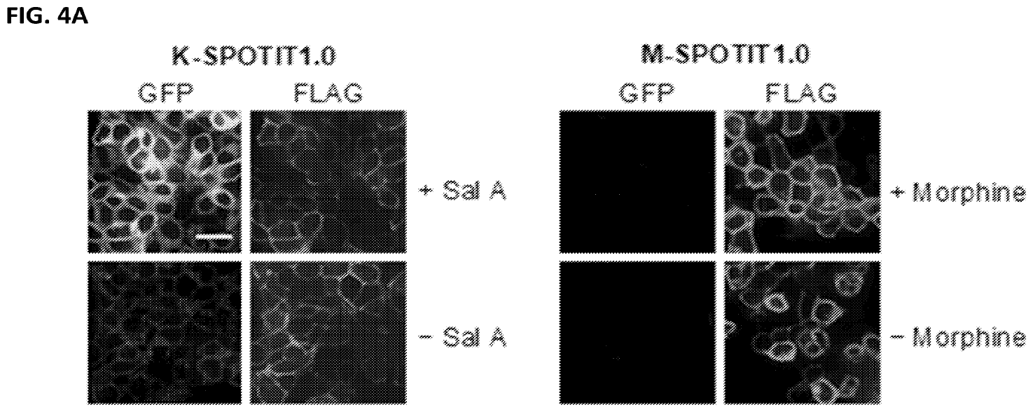

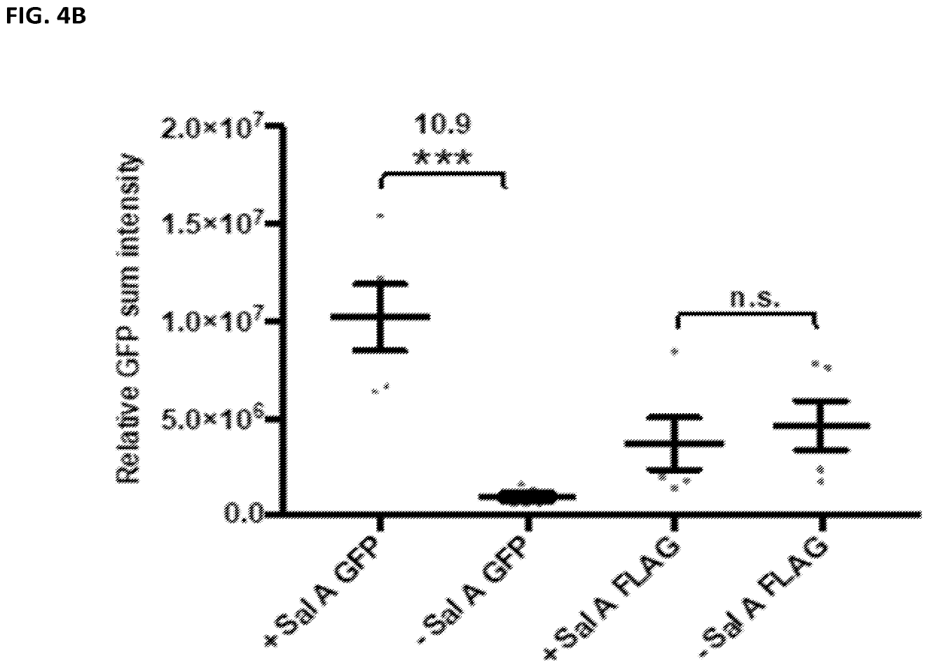

M-SPOTIT and the kappa-opioid receptor (KOR) version, K-SPOTIT, were designed and characterized. A FLAG tag was added at the extracellular side for characterizing the biosensor expression level. The opioid response of these two biosensors was first evaluated by monitoring their fluorescence immediately after addition of opioid agonists. Initially, a fluorescence increase was not observed; however, 24 hours after opioid agonist incubation, the KOR-biosensor showed 10.9-fold fluorescence increase in the presence of the synthetic KOR agonist, Salvinorin A (Sal A), compared to the condition without Sal A (FIG. 3b). However, the MOR-biosensor did not show any fluorescence increase (FIG. 4). These biosensors were named K-SPOTIT1.0 and M-SPOTIT1.0. To design a functional M-SPOTIT, the SPOTIT mechanism was evaluated first using K-SPOTIT1.0.

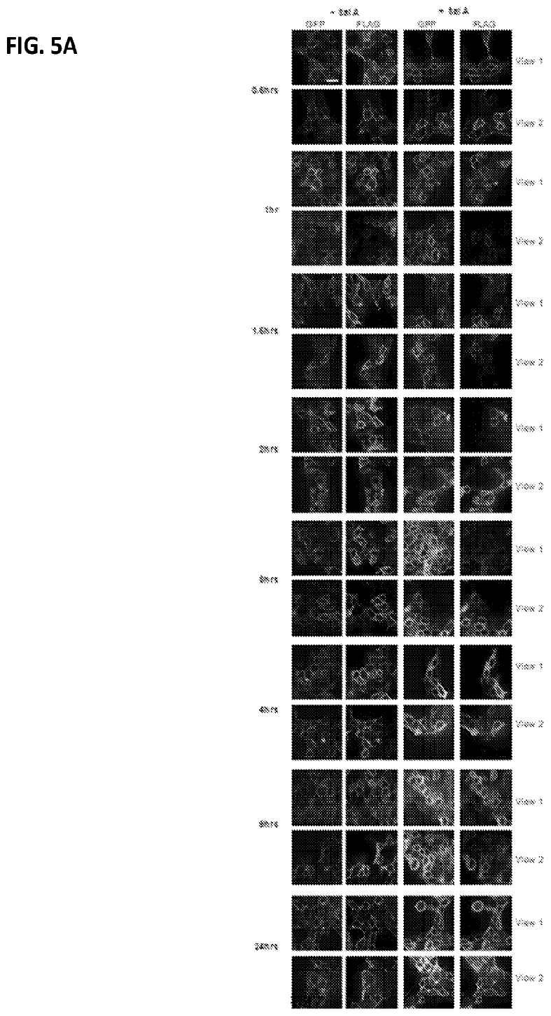

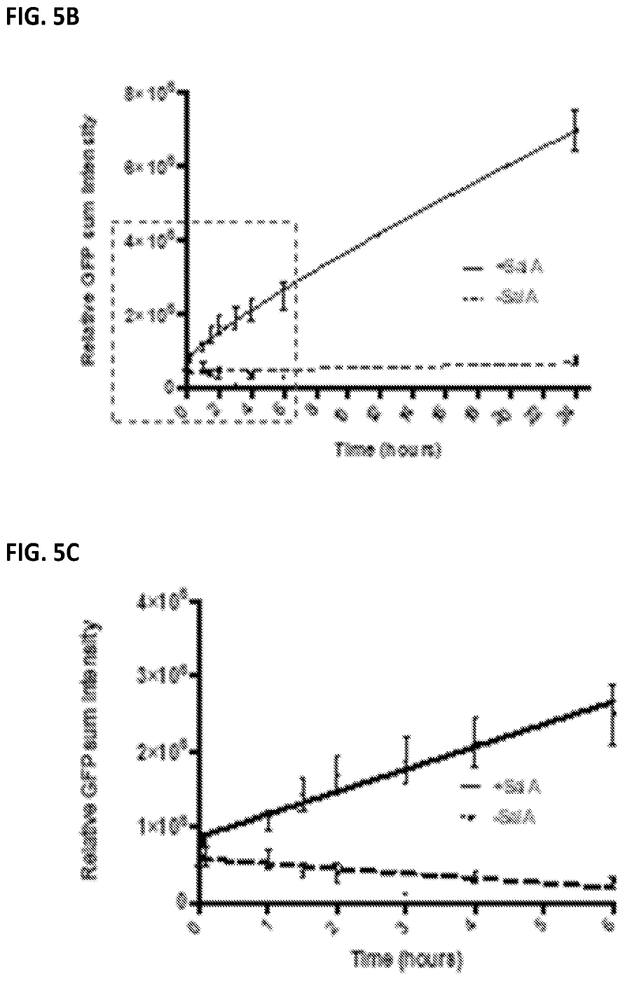

Because the fluorescence of K-SPOTIT1.0 did not increase immediately upon agonist addition as anticipated, the working mechanism of the K-SPOTIT1.0 fluorescence increase in response to KOR agonists was further evaluated. The biosensor's fluorescence increase at different time points with continuous agonist stimulation was investigated, and it was observed that the fluorescence gradually increased over a time course of 24 hours (FIG. 3c and FIG. 5).

This contradicted the initial biosensor design rationale that the fluorescence change would happen instantaneously. Because the change in the electronic environment surrounding the cpGFP fluorophore upon opioid-induced conformational change should be fast, it cannot be the cause of the fluorescence increase in K-SPOTIT1.0. Additionally, immunostaining of K-SPOTIT1.0 indicated the biosensor expression level is comparable (<1-fold change) in the presence and absence of Sal A (FIG. 3b and FIG. 4). This suggested the ˜ 11-fold fluorescence increase in the presence of opioid agonists is not due to elevated protein level or increased protein stability over time. Therefore, the slow rate of the fluorescence increase in the presence of the agonist is most likely due to fluorophore maturation, which takes approximately 40-minutes for EGFP¹⁶ and may be even longer for cpGFP. Based on these observations, it was hypothesized that the cpGFP's fluorophore in K-SPOTIT1.0 cannot mature in the basal state (without agonist); opioid agonist activation leads to the KOR-Nb39 bound state, allowing the fluorophore to mature.

Next, it was further investigated how the conformational state of the biosensor affects the matured biosensor fluorescence. The KOR antagonist, nor-binaltorphimine (Nor-BNI), was added to dissociate the Nb39 from KOR after 6 hours of agonist incubation, when the K-SPOTIT1.0 fluorophore is already matured. Comparable fluorescence was observed before and after addition of KOR antagonist (FIG. 6). Therefore, the conformational state after fluorophore maturation does not affect K-SPOTIT1.0 fluorescence.

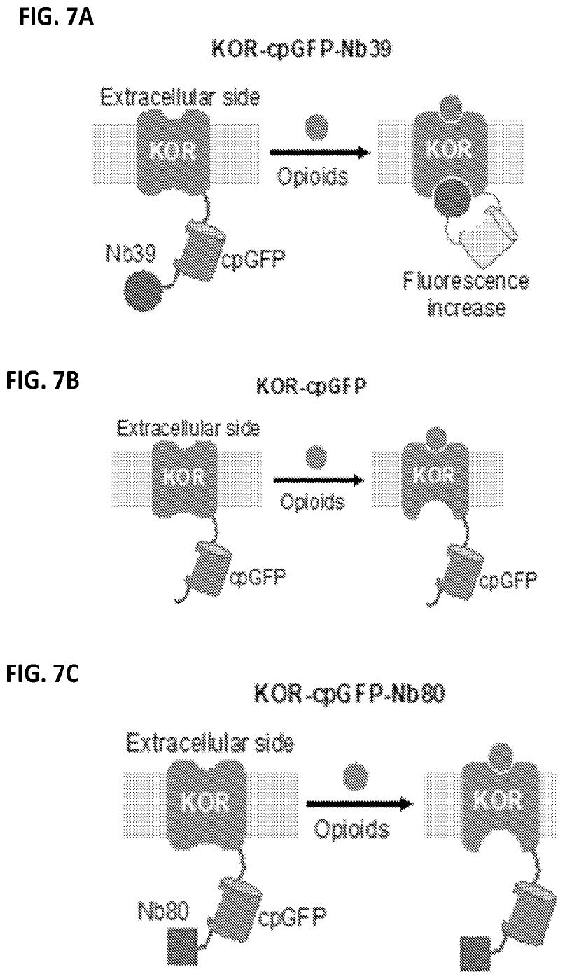

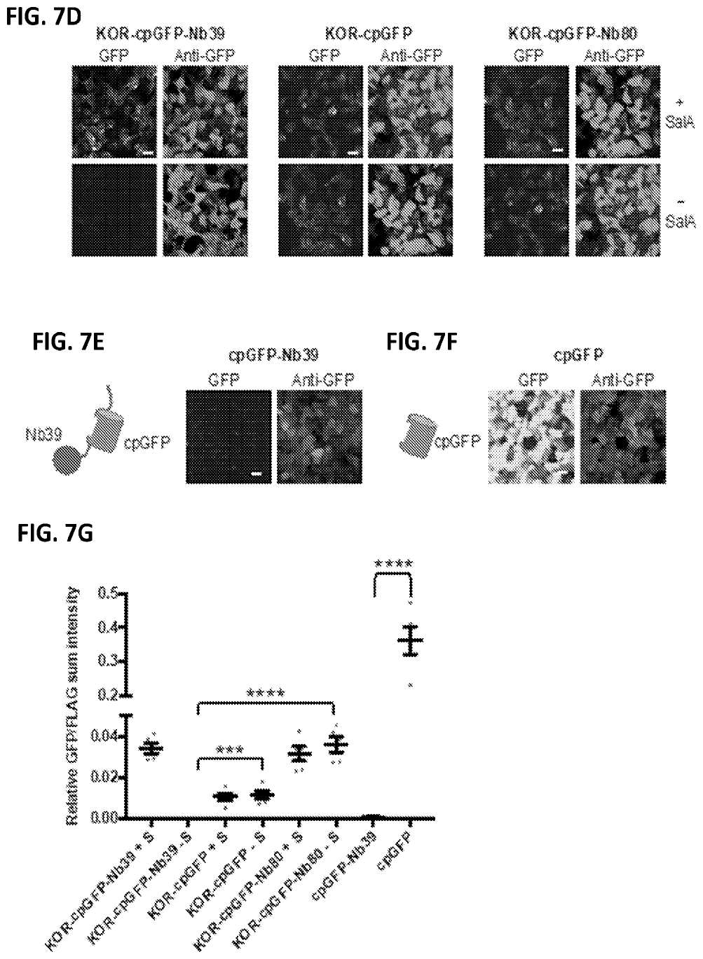

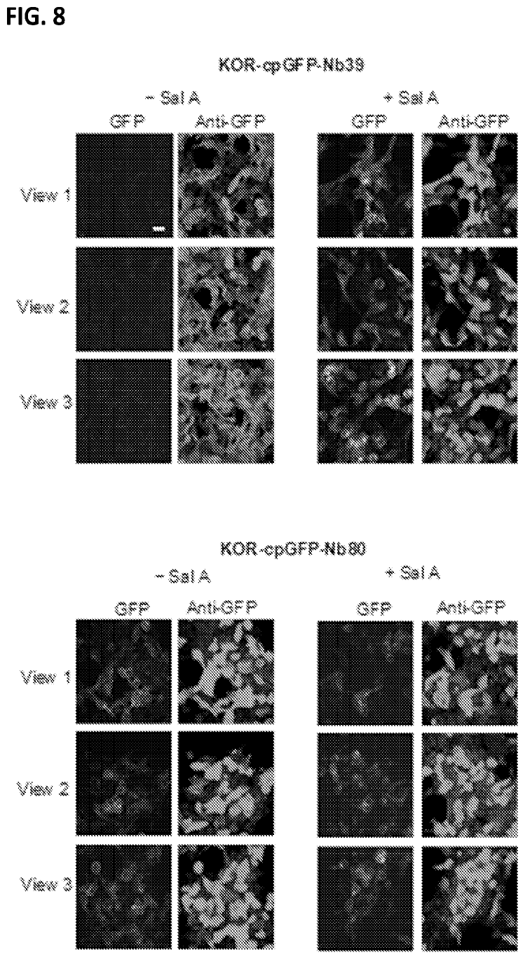

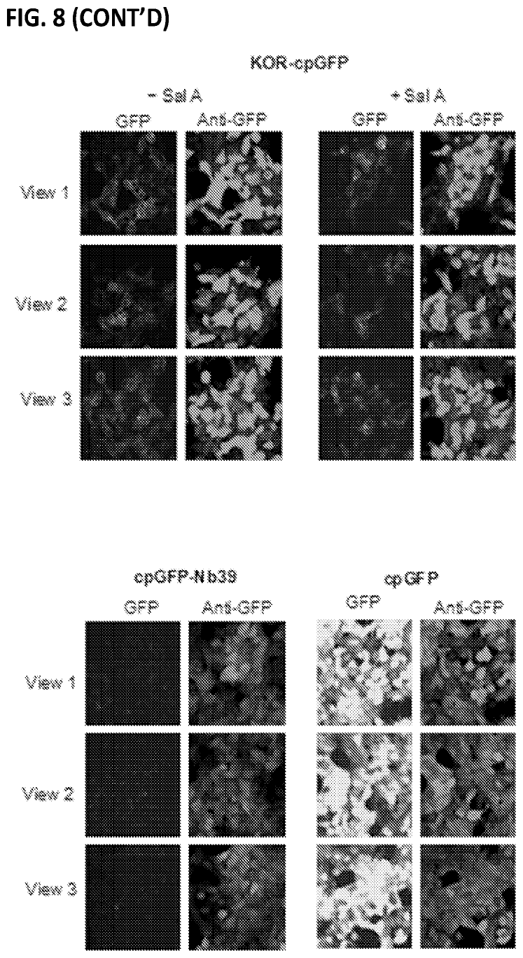

To further interrogate the necessity of the KOR-Nb39 bound state in the biosensor activation, Nb39 was deleted from K-SPOTIT1.0. Surprisingly, Nb39 deletion results in high background fluorescence in the absence of agonist (FIG. 7 and FIG. 8), indicating the fluorophore of cpGFP is already mature. Additionally, no fluorescence increase was observed after agonist incubation. This led us to further hypothesize that Nb39 is responsible for preventing the cpGFP chromophore from maturing in the basal state of K-SPOTIT1.0. Next, we changed Nb39 to Nb80, a G_{αs}-mimic nanobody (Rasmussen, S. G. F. et al. Structure of a nanobody-stabilized active state of the β2 adrenoceptor. Nature 469, 175-180 (2011), the entire contents of which are incorporated herein by reference for all purposes), which will not bind to the activated KOR. High background fluorescence was also observed, indicating that Nb39, but not Nb80, causes the inability of cpGFP's chromophore to mature (FIGS. 7c and 7d). To further test the generality of Nb39 in inhibiting cpGFP fluorophore maturation, soluble cpGFP-Nb39 and cpGFP alone were constructed (FIGS. 7e and f). cpGFP-Nb39 had low fluorescence signal while cpGFP alone had high fluorescence signal (FIG. 7e-g). These studies suggest that Nb39 interacts with cpGFP intramolecularly in a manner that prevents the chromophore from maturing; when KOR in K-SPOTIT1.0 is activated, Nb39 interacts with the receptor rather than cpGFP, allowing the fluorophore to mature.

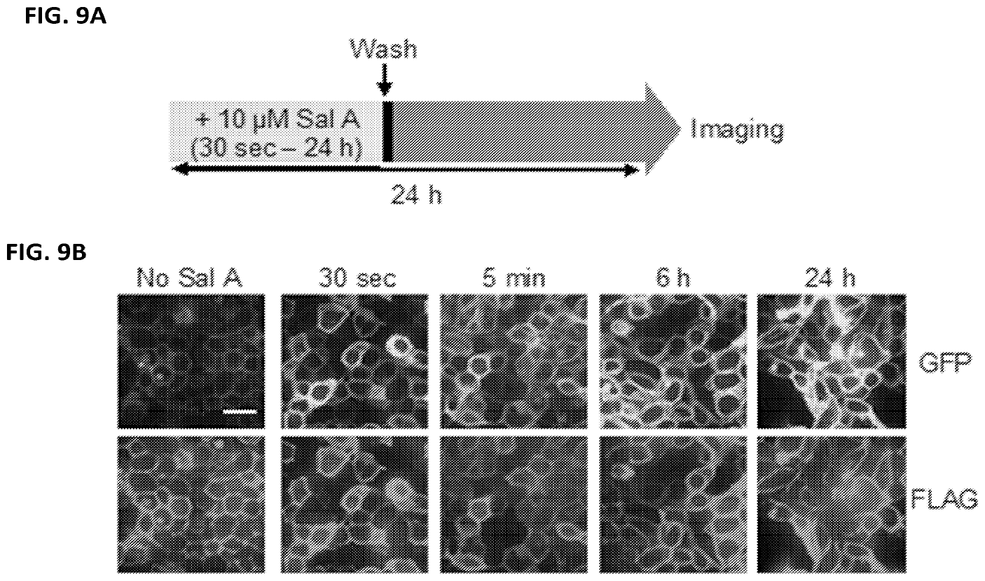

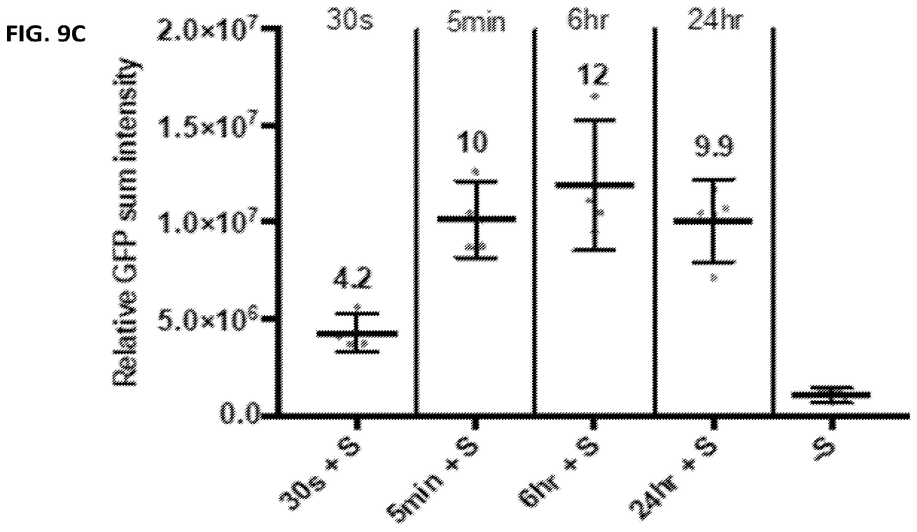

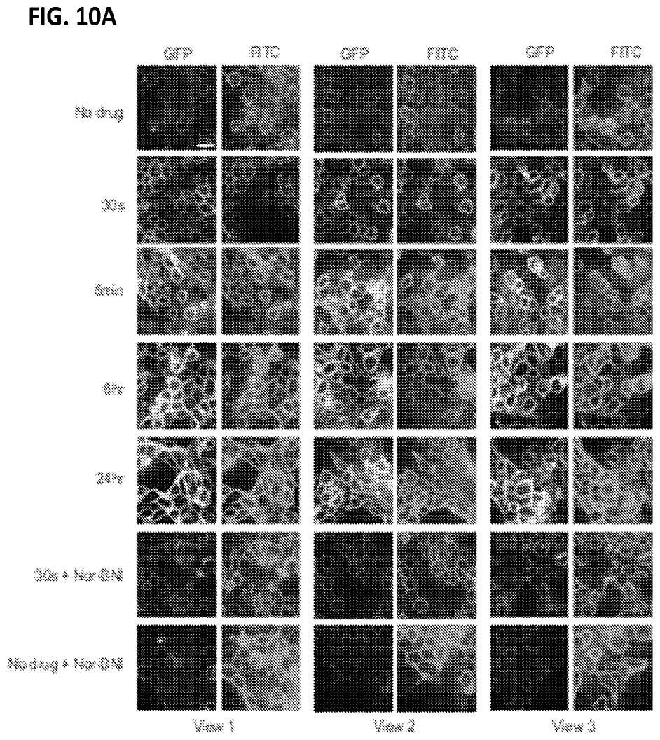

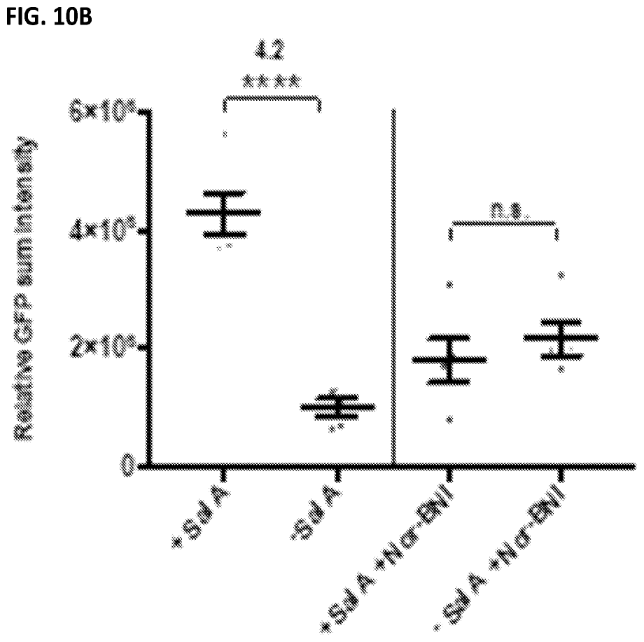

With a better understanding of the SPOTIT mechanism, the biosensor sensitivity to agonist exposure time was evaluated. The interaction between the activated KOR and Nb39 in SPOTIT was estimated to be enhanced due to their close proximity within a single protein chain. Consequently, the agonist-induced KOR-Nb39 bound state could remain stable even after the agonist is removed from the environment. As a result, a short-pulse of agonist exposure could also lead to biosensor activation as long as the biosensor is further incubated to allow fluorophore maturation. To test this, K-SPOTIT1.0 was treated with a short pulse of Sal A and then the agonist was removed. The cells were further incubated for a total of 24 hours (FIG. 9a). FIGS. 9b and c showed that a pulse of Sal A stimulation as short as 30-second is sufficient to activate K-SPOTIT1.0 with a 4.2-fold fluorescence increase, while longer incubation could lead to 12-fold fluorescence increase. This suggested that the agonist-induced KOR-Nb39 bound state can be stabilized after a brief opioid exposure; therefore, SPOTIT could be sensitive to short exposure of opioid agonists. To further test the importance of a stable KOR-Nb39 complex for SPOTIT activation, KOR antagonist, Nor-BNI, was added immediately after the initial 30-second agonist stimulation, and the cells were further incubated for 24 hours. Testing was conducted to determine whether antagonist addition would disrupt the KOR-Nb39 bound state of the biosensor, preventing the further maturation of the fluorophore. No significant fluorescence increase was observed if antagonist was added after the initial opioid exposure in comparison to Nor-BNI only cells (FIG. 10).

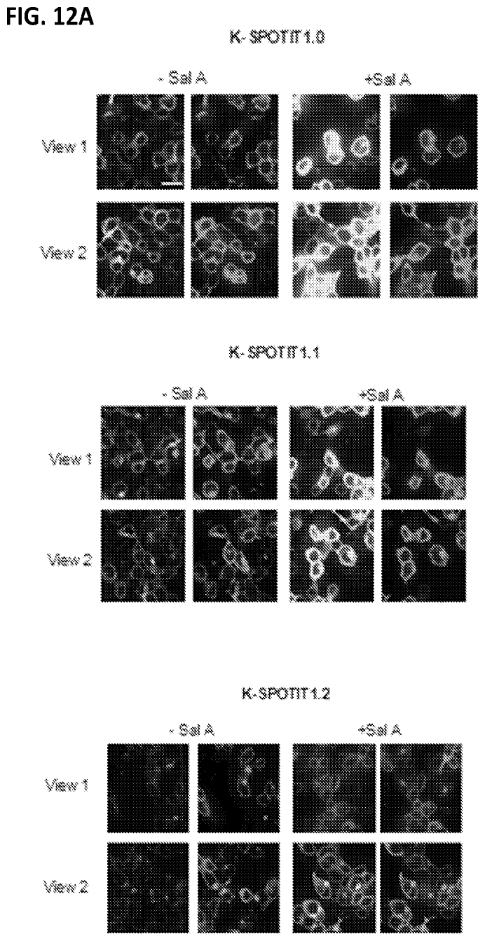

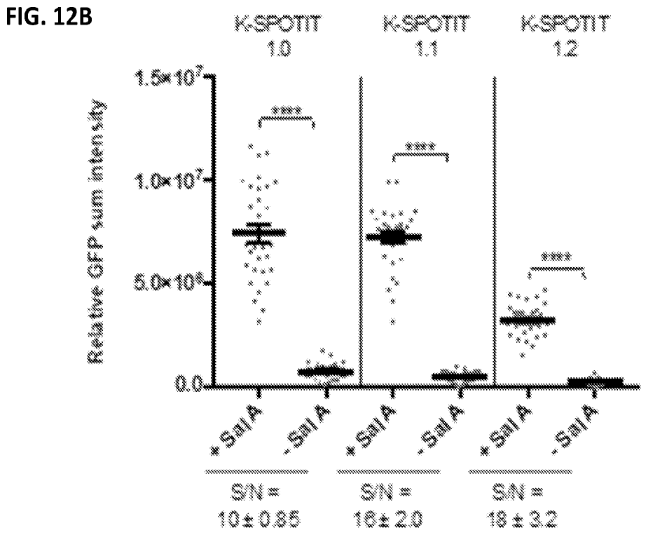

Lastly, to further optimize K-SPOTIT, the linkers connecting the KOR and cpGFP were varied. Additionally, the C-terminal domain of the KOR may play a role in its interaction with β-arrestin and subsequent endocytosis after receptor activation. To minimize the biosensor interaction with β-arrestin, different truncations of the intracellular C-terminal domain of the receptor after the palmitoyl cysteine (FIG. 11a) were made. The truncated version, K-SPOTIT1.1 with ten amino acids after the palmityl cysteine, showed higher fluorescence increase than K-SPOTIT1.0 and had comparable brightness to K-SPOTIT1.0 (FIG. 11b and FIG. 12). The more truncated form, K-SPOTIT1.2, led to a lower signal (FIG. 11b and FIG. 12), illustrating the importance of this linker for SPOTIT's function.

Design of functional M-SPOTIT. After dissecting the mechanism of SPOTIT, a functional M-SPOTIT was designed, because MOR is the opioid receptor most involved in pain-modulation and addiction. The first design of M-SPOTIT1.0 showed low background fluorescence, presumably because of the same inhibition of the cpGFP fluorophore maturation from Nb39. However, it did not show fluorescence increase upon agonist addition (FIG. 4). This is possibly because the cytosolic domain of MOR is not optimal to allow Nb39 to interact with the agonist-bound MOR. Nb39, therefore, cannot dissociate from cpGFP, and this inhibits cpGFP fluorophore maturation. Because the cytosolic domain of KOR is effective in K-SPOTIT1.1, a chimeric biosensor for M-SPOTIT was generated by replacing the cytosolic domain of the MOR with that of K-SPOTIT1.1 (FIG. 11c). This chimeric design, which was named M-SPOTIT1.1, yielded a 3.2-fold fluorescence increase upon incubation with MOR agonists (FIG. 11d).

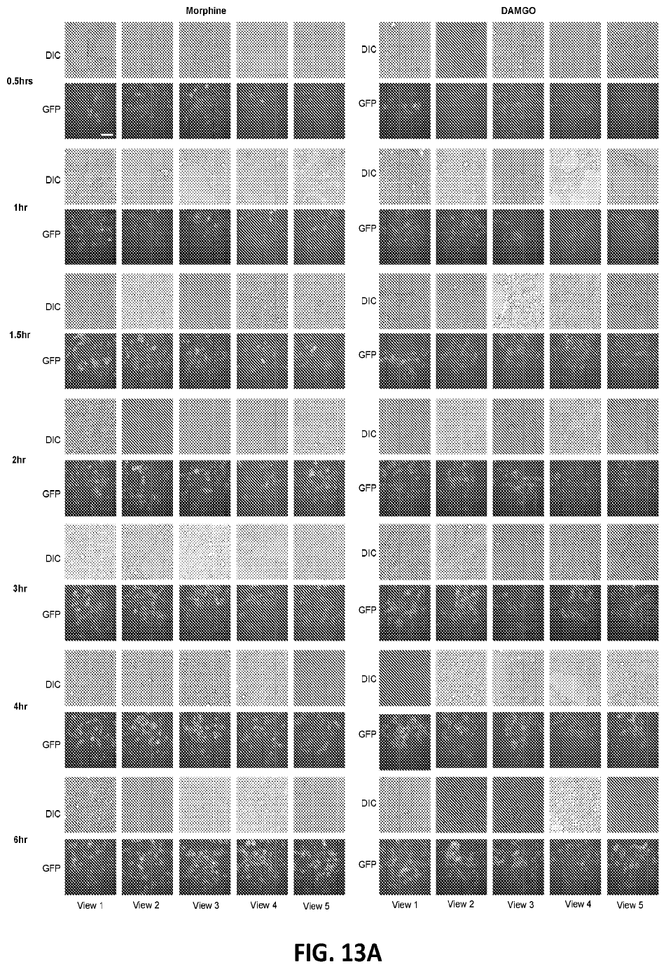

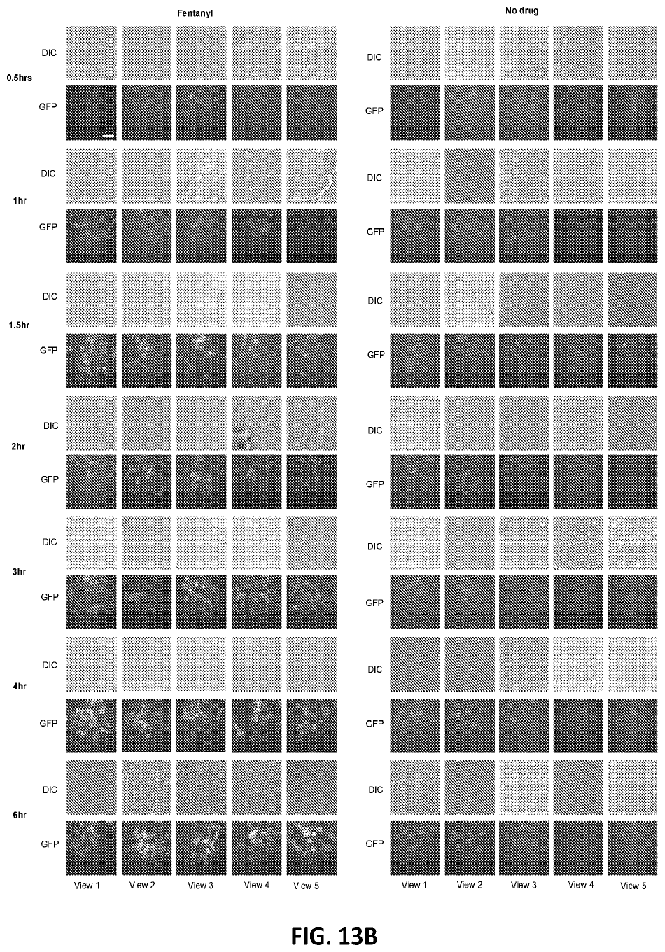

Similar to K-SPOTIT1.0 characterization, the biosensor maturation time for M-SPOTIT1.1 was evaluated under continuous opioid stimulation. HEK293T cells expressing M-SPOTIT1.0 were incubated with MOR agonists, including morphine, DAMGO and fentanyl, for 30 minutes to 6 hours. Approximately a 6.0-fold biosensor fluorescence increase was observed at 2 hours for fentanyl and morphine-treated cells, and the fluorescence continued to increase with longer agonist incubation time (FIG. 11e and FIG. 13A-13B). The continuous increase of fluorescence over time could be due to a combination of gradual fluorophore maturation and continuous activation of the newly-translated biosensors. DAMGO showed lower biosensor activation than fentanyl, possibly because DAMGO is not cell permeable, while fentanyl is cell-permeable and, therefore, can activate the biosensor population trapped in the secretory pathway.

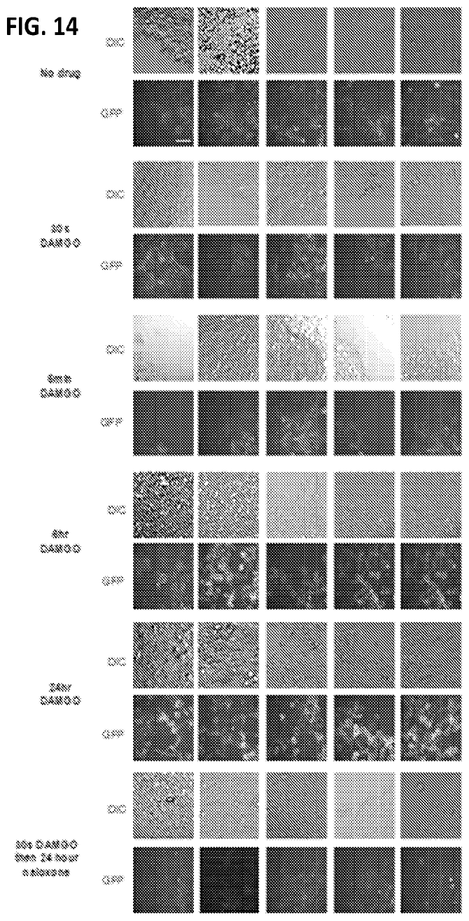

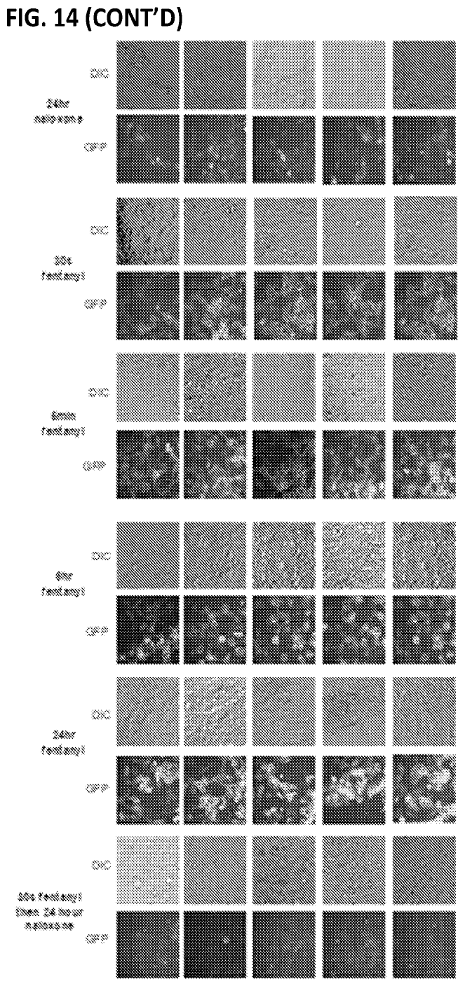

M-SPOTIT1.1's sensitivity to short pulses of agonist stimulation followed by further incubation without agonist was tested. M-SPOTIT1.1 was stimulated with a short-pulse of agonist exposure, followed by drug removal and further incubation for 24 hours (FIG. 4f). Similar to K-SPOTIT, 30 seconds of agonist exposure is sufficient to generate a robust fluorescence signal for M-SPOTIT1.1 with potent agonists, like fentanyl. However, DAMGO requires longer incubation time for activation of the biosensor (FIG. 11f and FIG. 14).

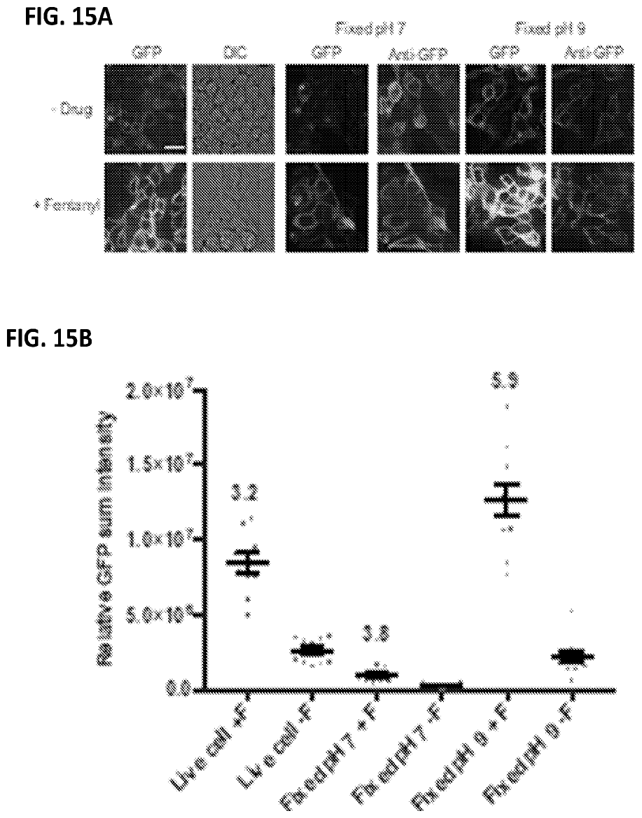

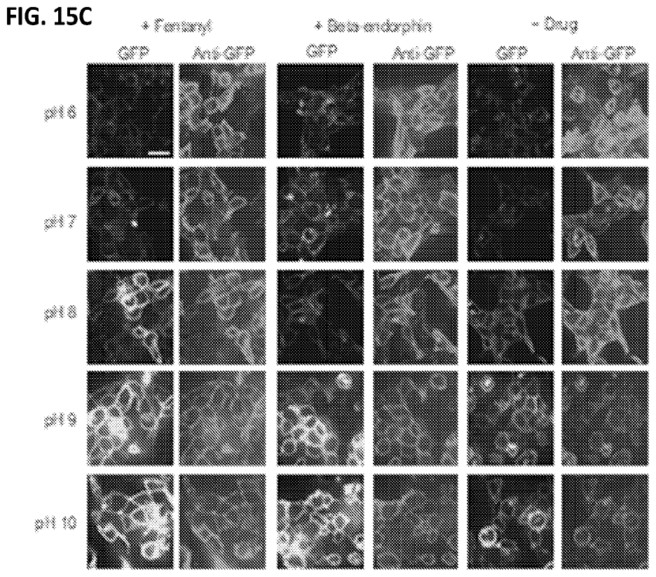

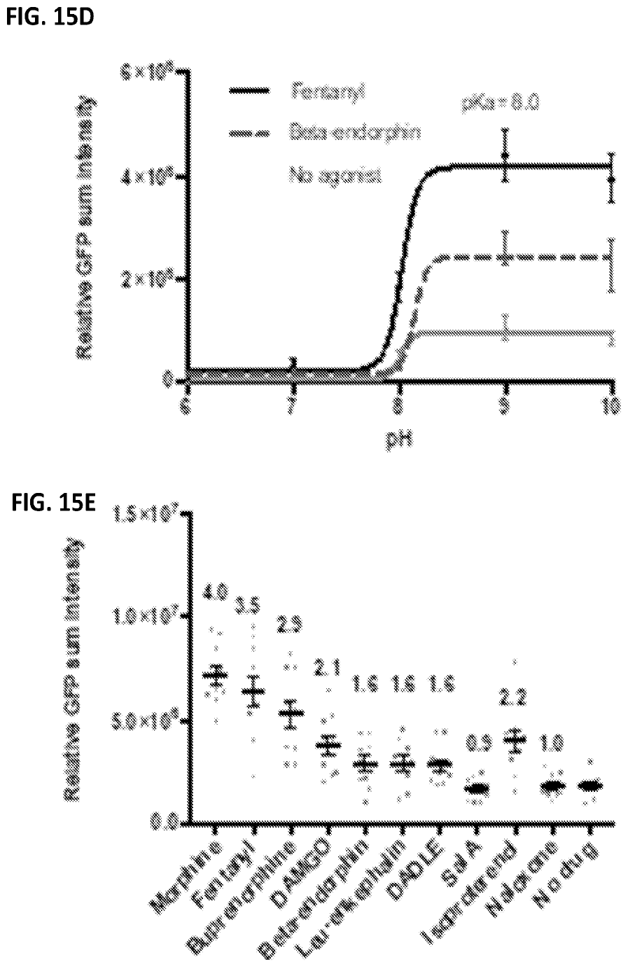

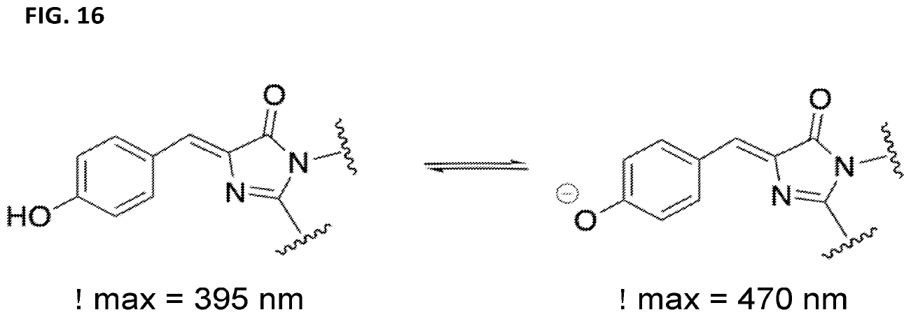

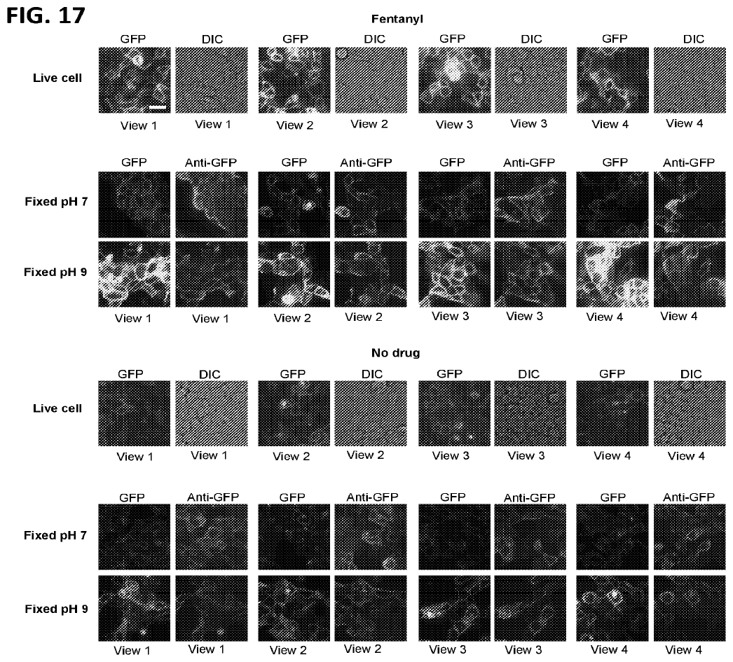

Further characterization of M-SPOTIT1.1 by immunostaining showed that when fixed at pH 7.0, M-SPOTIT1.1's fluorescence significantly decreased (FIGS. 15a and 15b). Since the fluorophore of M-SPOTIT was already formed, the decrease of the fluorescence could be due to the protonation of the fluorophore, which is less fluorescent than the deprotonated form (FIG. 16 Presumably, formaldehyde cross-linking could cause a higher fluorophore pKa or change the pH surrounding the fluorophore. To test whether the fluorescence decrease is due to fluorophore protonation, HEK293T cells were transfected with M-SPOTIT1.1 and the fluorescence in live cells or fixed cells at pH 7 and 9 was compared. FIG. 15b shows that the fentanyl-activated M-SPOTIT1.1 was 13.8-fold higher at pH 9 than at pH 7 in fixed cells. A S/N of 3.2 was observed for live cells, 3.8 for fixed cells at pH 7, and 5.9 for fixed cells at pH 9 (FIG. 15a, 15b, and FIG. 17). This validated the hypothesis that cpGFP in M-SPOTIT1.1 is in its protonated state at pH 7 in fixed cells.

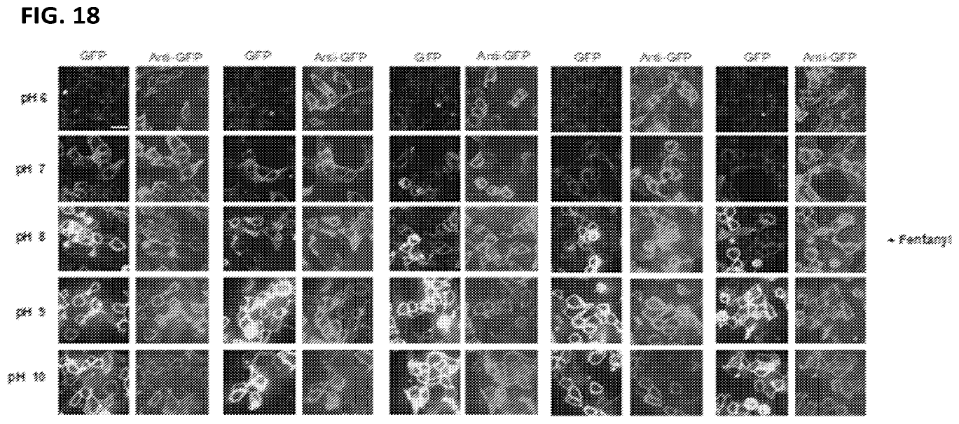

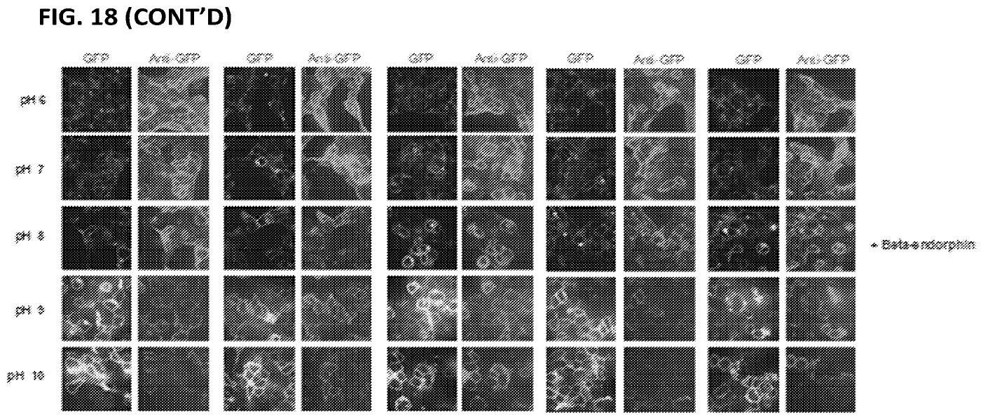

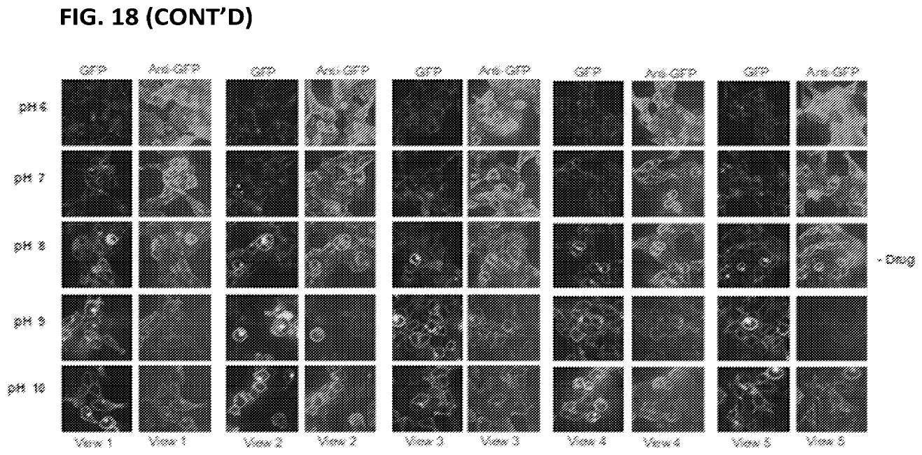

To determine the pKₐ of the fluorophore, a pH titration was performed for M-SPOTIT1.1 in the following three states: the basal state and the beta-endorphin and fentanyl-activated states. Based on the titration curves, the pKₐ of the fluorophore was determined to be 8.0 in fixed cells (FIGS. 15c, d, and FIG. 18). Therefore, when imaging M-SPOTIT1.1 in formaldehyde-fixed cells, the image should be taken at a pH>8 to observe optimal fluorescence signal.

Since the cpGFP fluorophore at pH 10 should be fully deprotonated and at its most fluorescent state, the different fluorescence observed for M-SPOTIT1.1 with or without agonist indicates different amount of the fluorophore formed. This further supported the hypothesis that the SPOTIT mechanism is based on fluorophore maturation.

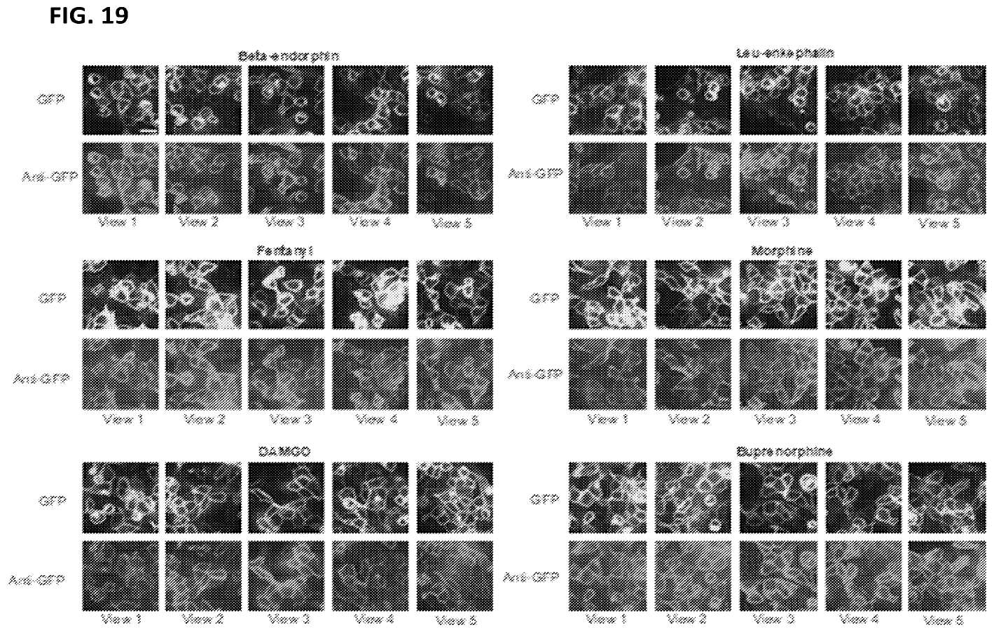

Next, the selectivity of M-SPOTIT1.1 for MOR agonists was characterized. M-SPOTIT1.1 was incubated with various drugs, including MOR peptide agonists, partial and full synthetic MOR agonists, and antagonists. FIG. 15e and FIG. 19 showed that MOR agonists, such as morphine, fentanyl, buprenorphine, DAMGO, and the peptides leu-enkephalin and beta-endorphin activate M-SPOTIT1.1, illustrating its versatility for a variety of MOR agonists. Interestingly, DADLE, an agonist for the delta-opioid receptor, could also activate M-SPOTIT1.1, showing a 1.6-fold increase in fluorescence. This is consistent with previous studies that suggest DADLE has a weak affinity for MOR. Even more interestingly, isoproterenol activated M-SPOTIT as well, producing a 2.2-fold signal change. This is also consistent with previous studies that show beta-adrenergic receptor agonists can have activity towards opioid receptors. This shows that M-SPOTIT1.1's response to different drugs is positively correlated to the drug's ability to activate MOR.

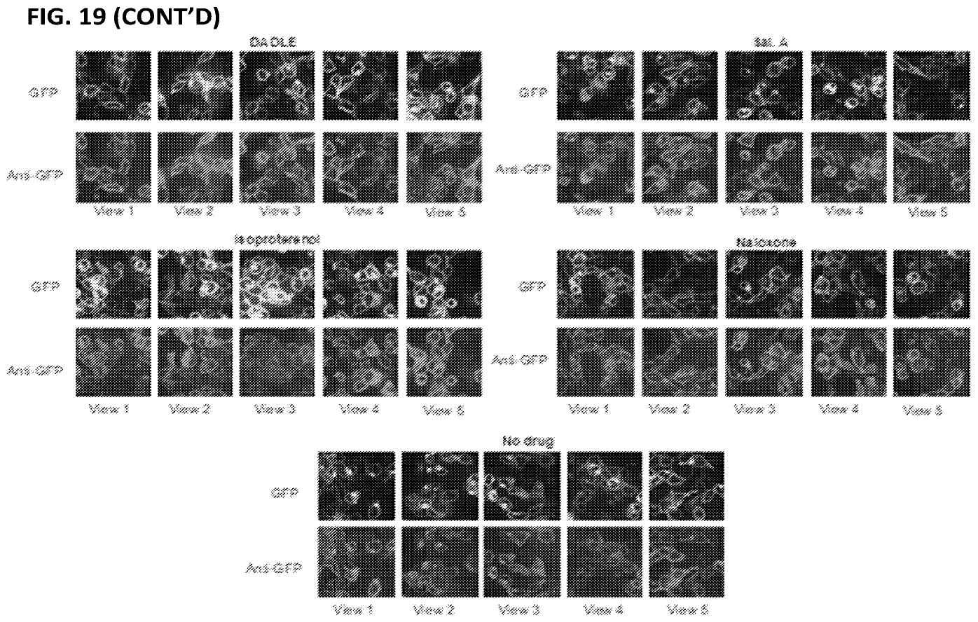

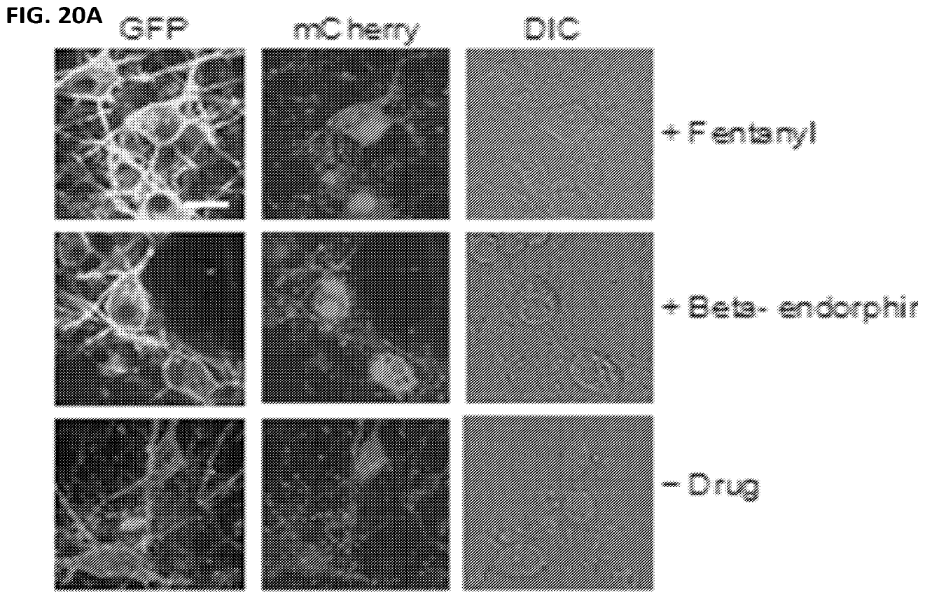

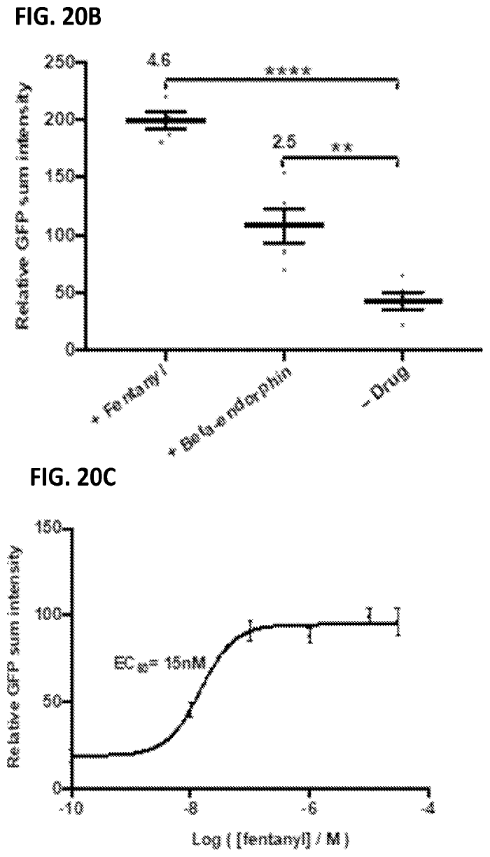

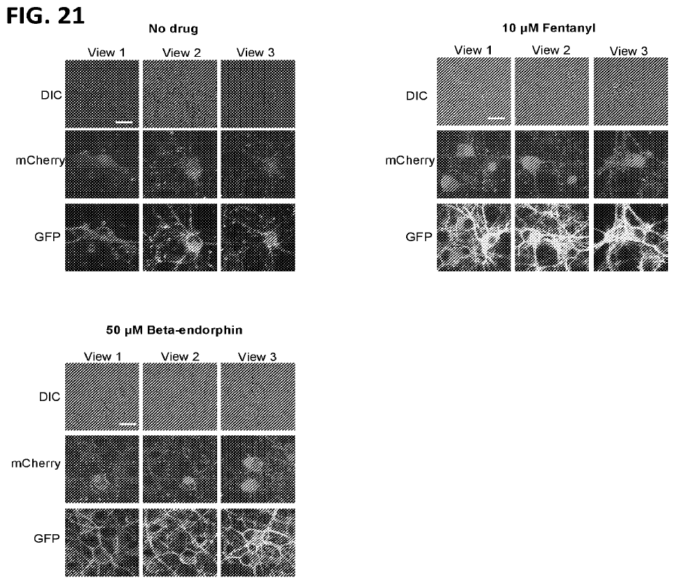

Testing M-SPOTIT1.1 in cultured neurons. M-SPOTIT1.1 can potentially be used to determine the site-of-action of endogenous and exogenous opioids in an animal brain. To test the feasibility this application, M-SPOTIT1.1 was expressed in cultured neurons by AAV viral infection. Stimulation with fentanyl and beta-endorphin led to a 4.6-fold and 2.5-fold activation of M-SPOTIT1.1, respectively (FIG. 20a, 20b, and FIG. 21). The lower S/N of beta-endorphin could possibly be due to the degradation of the peptide or the inability of the peptide to activate biosensors not expressed on the cell membrane.

To compare an agonist's binding affinity to M-SPOTIT versus MOR, a fentanyl titration was performed in cultured neurons. M-SPOTIT1.1 had an apparent EC₅₀ of 15 nM for fentanyl, which is comparable to the reported IC₅₀ value of fentanyl (8.4 nM) for MOR expressed in HEK293T cells (FIG. 20c and FIG. 22). This means fentanyl has a similar binding affinity to M-SPOTIT1.1 as MOR.

In sum, this example provides a genetically-encoded biosensor for detecting opioids in cultured neurons. This shows that M-SPOTIT1.1 finds use for detecting opioids in the brain. M-SPOTIT1.1 can also be expressed in glial cells under a CAG biosensor. Biosensor expression and performance in glial cells will more closely resemble HEK293T cells, enabling the detection of endogenous and exogenous opioids.

### Discussion

To enable the detection of opioids for MOR at a high spatial resolution, M-SPOTIT1.1 was designed. M-SPOTIT1.1 has many advantageous characteristics. First, M-SPOTIT1.1 has a S/N up to 9.8-fold and is selective for MOR agonists, allowing a new high-throughput approach to detect opioid agonists in cell cultures. M-SPOTIT1.1's activation is positively correlated to the concentration of the opioid agonists and can detect fentanyl with an EC₅₀ of 15 nM in cultured neurons. A higher-affinity Nb39 variant may also be used in the GEFIs described herein, that may stabilize the agonist-bound receptor.

M-SPOTIT1.1 utilizes a new mechanism to integrate the transient opioid signal to a persistent fluorescent signal, making M-SPOTIT1.1 sensitive to short pulses of opioid stimulation and enabling image analysis at high spatial resolution in fixed cells. This study showed that the fluorophore maturation of cpGFP in M-SPOTIT1.1 is inhibited by Nb39 in the basal state; opioid agonist-induced intramolecular MOR-Nb39 complex formation allows the cpGFP fluorophore to mature and generate a persistent green fluorescence signal for image analysis. This mechanism was further validated by imaging the agonist-induced biosensor fluorescence change at pH 10, where the fluorophore pKₐ should not have an effect on the S/N. The signal change observed with addition of agonist is, therefore, solely due to fluorophore maturation. This represents a new mechanism of biosensor design, which can be applied to design other GPCR biosensors. Due to this unique biosensor mechanism, M-SPOTIT1.1 can be sensitive to a short-pulse of stimulation when a strong opioid agonist, such as fentanyl, is applied. This is because a stable OR-Nb39 complex can form after a 30-second opioid stimulation and persists, allowing for further fluorophore maturation.

M-SPOTIT1.1 is the first single protein chain opioid biosensor and only requires one DNA construct for expression, making its performance less protein expression dependent. Therefore, compared to multiple component biosensors such as Tang or split luciferase assay, it will be easier to be express M-SPOTIT1.1 in cell cultures and animal models, and M-SPOTIT1.1's performance is contemplated to be more consistent. M-SPOTIT1.1 can be expressed in neurons for detecting opioids in the brain. It can also be expressed in glial cells under a CAG promoter, therefore enabling a higher biosensor expression level and signal to determine the localization of opioids in the brain.

Overall, M-SPOTIT1.1 represents the first demonstration of a genetically-coded tool to detect opioid agonists for MOR in neurons at a cellular resolution. M-SPOTIT1.1 finds use for screening and characterizing synthetic opioid agonists in cell cultures and finds use for detecting opioids at a cellular resolution in animal models to study the localization of exogenous and endogenous opioids.

### Example 2

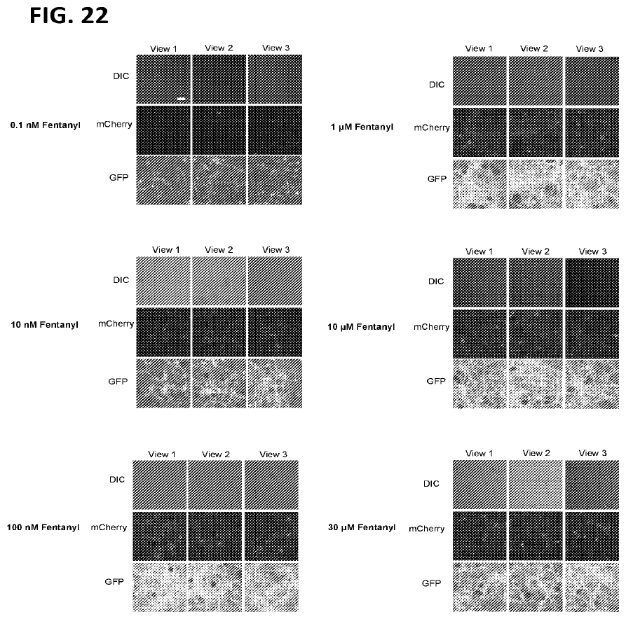

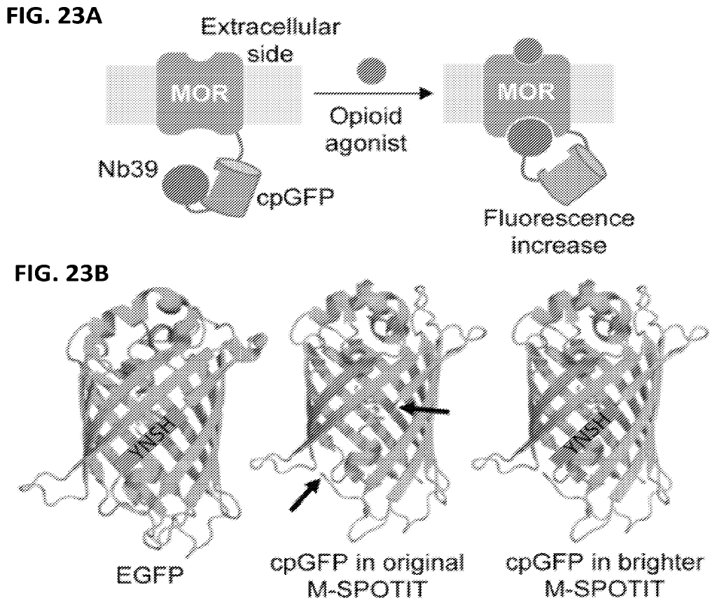

M-SPOTIT1.1 described in Example 1 has a unique fluorescence activation mechanism that is based on the fluorophore maturation of the circular permuted green fluorescent protein (cpGFP). As shown in FIG. 23A, in the absence of an opioid receptor agonist, a Gai mimic nanobody, Nb39 interacts with cpGFP, inhibiting its fluorophore from undergoing a series of maturation steps necessary for fluoresence. Upon activation of the opioid receptor by an agonist, Nb39 binds to the intracellular portion of the opioid receptor, releasing cpGFP and allowing its fluorophore to mature. This results in an irreversible fluorescence increase upon opioid detection.

M-SPOTIT1.1 has a good signal-to-noise ratio (SNR, defined as the ratio between the fluorescence in the presence and absence of opioids). To improve the brightness of M-SPOTIT1.1, the crystal structure of cpGFP in comparison to enhanced GFP (EGFP) was closely examined. As shown in FIG. 23B, four amino acids, YNSH, which are located near the fluorophore of EGFP are missing in cpGFP. Accordingly, two new versions of M-SPOTIT1.1 were engineered, the four amino acids YNSH were added to either the N- or C-terminus of cpGFP to enhance its brightness (FIG. 23B). The version where YNSH is added to the N-terminus was named “YNSH-MSPOTIT” and the version where YNSH is added to the the C-terminus was named “MSPOTIT-YNSH”.

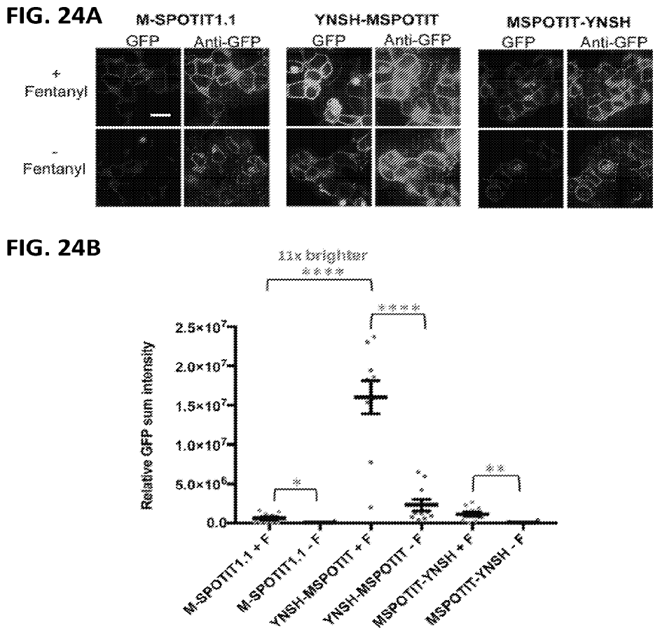

It was tested whether these new sensors in HEK293T cells 24 hours post opioid stimulation allow the fluorophore to fully mature, and their brightness was compared to the original sensor version, M-SPOTIT1.1. HEK293T cells were then fixed and imaged at pH 11. Fixation uses formaldehyde to cross-link proteins, resulting in cell death and cellular organelle and protein preservation. This allowed for imaging the cells at pH 11, where the fluorophore is in its fully deprotonated state. Confocal imaging analysis showed a 11× higher brightness for YNSH-MSPOTIT compared to the original M-SPOTIT1.1 with the brightness normalized to a protein expression marker (FIGS. 24A and 24B). Because YNSH-MSPOTIT is brighter at pH 11, where the cpGFP fluorophore is at its fully deprotonated state (fluorescent state), the increase of brightness is either due to a larger amount of matured fluorophore, a brighter fluorophore from greater beta-barrel encapsulation, or a combination of both factors. MSPOTIT-YNSH, however, was not significantly brighter than M-SPOTIT1.1. Therefore, YNSH has a larger effect when added to the N-terminus of cpGFP. This could be because the N-terminus of cpGFP forms a beta-sheet that composes part of the beta-barrel. Consequently, the N-terminus is more structured and rigidified than the C-terminus of cpGFP which forms a less structured loop. The addition of YNSH to the N-terminus, therefore, will be more structured while addition to the C-terminus might result in the inability of YNSH to fold back to complete the beta-barrel.

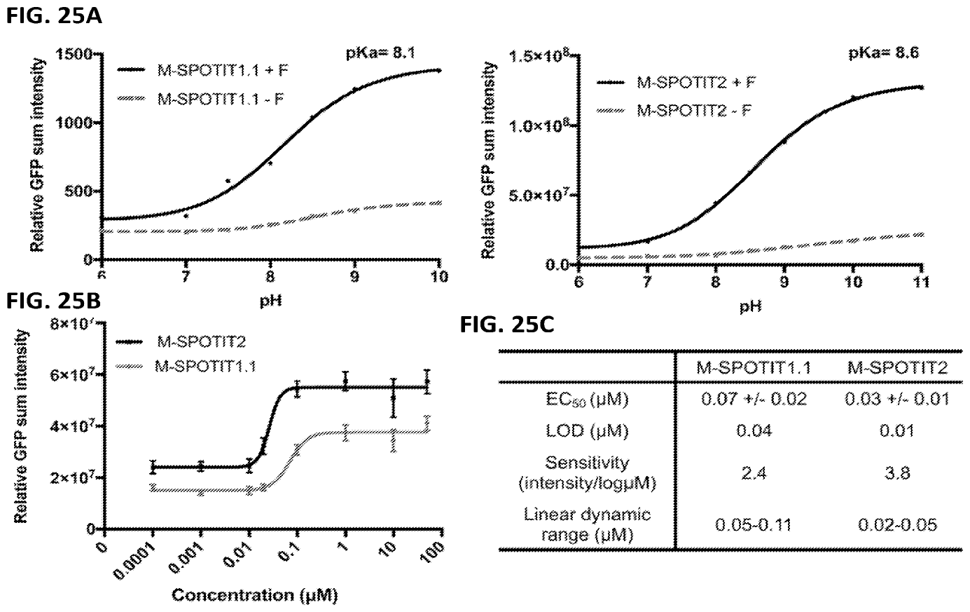

Despite being brighter, YNSH-MSPOTIT has a lower SNR, 7, than M-SPOTIT1.1's SNR which is 25. The lower SNR is due to the higher background signal, possibly caused by the disruption of the interaction between Nb39 and cpGFP. Nb39 presumably interacts with the opening in cpGFP's beta-barrel, and the addition of YNSH might sterically block Nb39 from interacting with the cpGFP fluorophore, thereby lowering Nb39's fluorophore inhibition efficiency. Despite the lower SNR, the 11× brighter signal of YNSH-MSPOTIT compared to M-SPOTIT1.1 makes YNSH-MSPOTIT extremely useful for experiments that require a bright signal to detect opioids. This brighter sensor was named “M-SPOTIT2”. Formaldehyde fixation raises the pKa of the cpGFP fluorophore, making it necessary to image M-SPOTIT1.1 with a high pH buffer (pH 4 9) after fixation or image live-cell at physiological pH.3 To fully characterize M-SPOTIT2, its pKa was evaluated in fixed cells by performing a pH titration of M-SPOTIT2 and M-SPOTIT1.1. The pKa of M-SPOTIT2 shifted to 8.6 compared to 8.1 for M-SPOTIT1.1 (FIG. 25A). Therefore, the new sensor should be imaged in fixed cells at a pH 4 9.6 or in live-cells. Additionally, M-SPOTIT2 was further compared to M-SPOTIT1.1 by determining the limit of opioid detection (LOD), sensitvity, EC50, and dynamic range values for both sensors. To do so, a titration curve with the full MOR agonist, fentanyl, was performed. The improved M-SPOTIT2 showed a comparable LOD and sensitivity to the original M-SPOTIT1.1 and a lower EC50 value (FIGS. 25B and 25C).

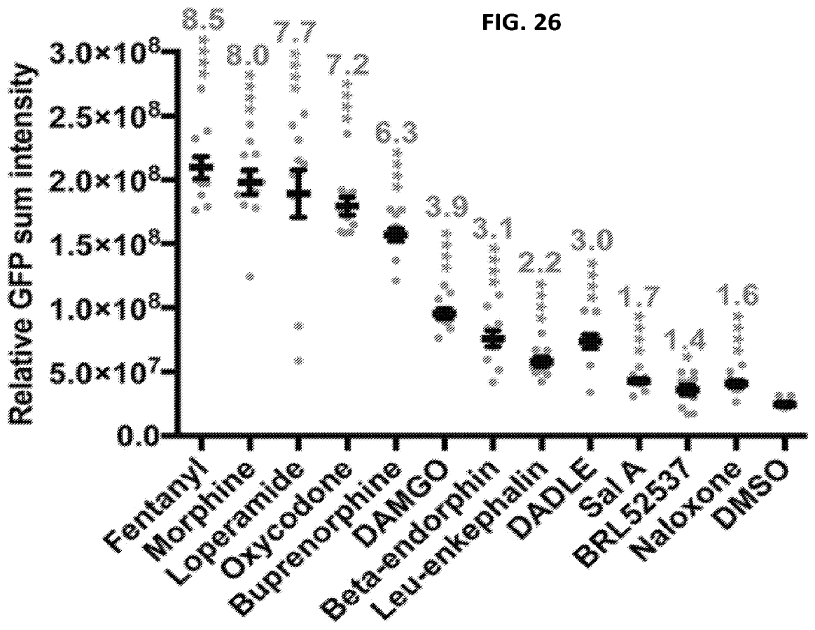

Current methods to detect opioids in brain tissue, such as nano-flow liquid chromatography mass-spectrometry, can only determine concentrations of opioids in a large volume of brain tissue but cannot be used to determine concentrations at cellular resolution. Additionally, methods with improved spatial resolution, such as fast scan cyclic voltammetry, have only been used to detect met-enkephalin and not other opioid peptides. M-SPOTIT is the first development of a tool that can detect MOR agonists at cellular resolution. To characterize the drug selectivity of M-SPOTIT2 and illustrate its use in HTS of MOR agonists, M-SPOTIT2 was tested against a variety of different drugs, including MOR synthetic full agonists, partial agonists, peptide agonists, kappa opioid receptor (KOR) agonists, and an antagonist (FIG. 26). Significant signal changes for all MOR agonists in comparison to a DMSO and media vehicle were seen. Further, the SNR is positively correlated to the potency of the agonist with synthetic agonists (fentanyl, morphine, loperamide, oxycodone, and buprenorphine) giving the highest SNR. KOR agonists (SalA, BRL52537) and MOR antagonist (naloxone) showed a much lower SNR than the MOR agonists. This illustrates that M-SPOTIT2 is selective towards MOR agonists.

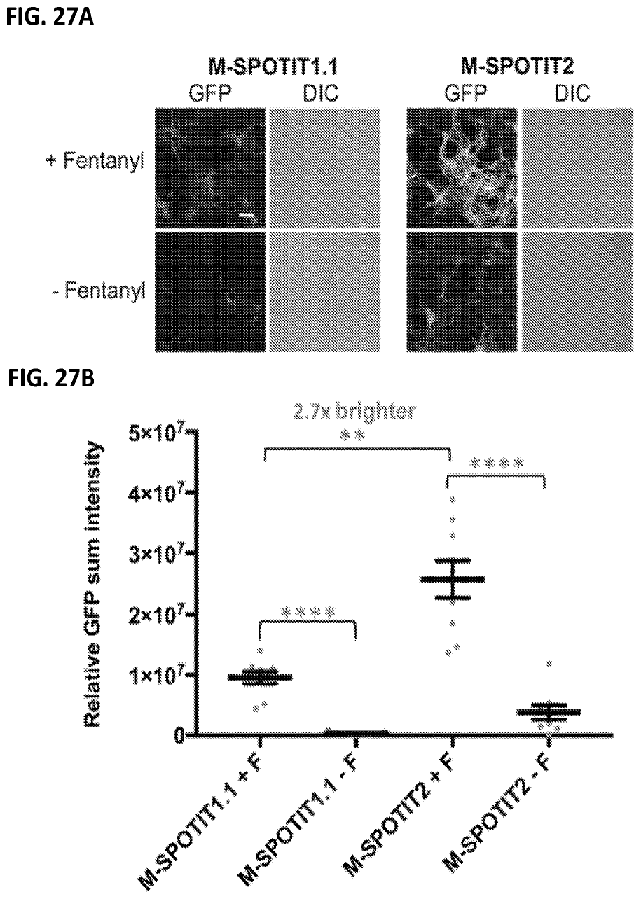

To further assess M-SPOTIT2's feasibility as a HTS platform, its Z-factor was calculated. This value gives information about the robustness and reproducibility of the platform. A Z-factor greater than 0.5 is considered an excellent value for HTS. M-SPOTIT2 has a Z-factor of 0.548, illustrating its usefulness as a HTS assay. Current methods for MOR drug screening in cell culture are limited by low-throughput, costly reagents, or b-arrestin-2 pathway dependence. M-SPOTIT2 as an HTS platform would be cost effective, high throughput, and G-protein dependent. Further, the 11× brighter signal for M-SPOTIT2 will allow a wide-array of agonists with different affinities towards MOR to be detected, enabling the discovery of novel ligands for MOR. To illustrate the potential use of M-SPOTIT2 in the animal brain, adeno-associated virus (AAV) infection was used to express M-SPOTIT2 in rat cortical neuronal culture (FIG. 27). M-SPOTIT2 showed significant opioid-dependent fluorescence increase (SNR=6.8) and is 2.7× brighter than the original M-SPOTIT1.1 (SNR=27). This brighter sensor will overcome the autofluorescence in brain tissues and, therefore, be more robust for opioid detection in animal models.

In summary, M-SPOTIT2 with the four amino acids YNSH added to the N-terminus of the cpGFP in M-SPOTIT1.1 is brighter in both HEK293T cell and neuronal cultures. Even though M-SPOTIT2 has an opioid-dependent SNR up to 8.5, its SNR can still be improved by lowering its background fluorescence in the absence of MOR agonists. The increased background fluorescence of M-SPOTIT2 could be partially due to a weakened interaction between Nb39 and cpGFP due to the addition of YNSH to cpGFP. M-SPOTIT2 will be a useful tool for both HTS of opioids and detection of endogenous and exogenous opioids in an animal brain at cellular resolution.

### Example 3

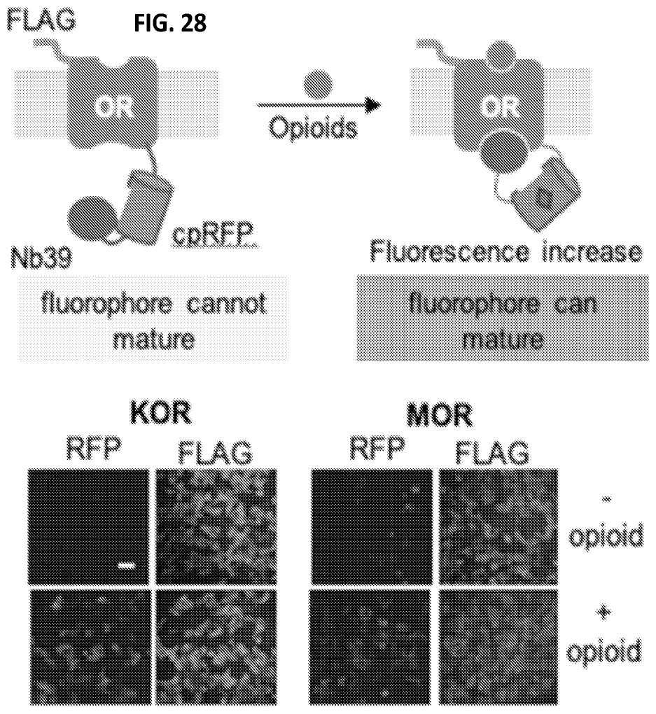

A red fluorescent protein version of the opioid biosensor, red-SPOTIT, was engineered for both the kappa and mu opioid receptors (KOR and MOR, respectively) by using a circularly permuted red fluorescent protein (cpRFP) instead of cpGFP. A schematic of the red-SPOTIT is shown in FIG. 28A. Nb39 can still inhibit cpRFP fluorophore maturation, meaning red-SPOTIT follows the same mechanism as the cpGFP version (FIG. 28B).

The red and green versions of the opioid biosensors described herein can be used for multiplexed imaging of opioid agonists for multiple opioid receptors at one time. For an example, a red fluorescent protein-based KOR sensor and a green fluorescent protein-based MOR sensor can be used to detect agonists for KOR and MOR at the same time in an animal brain. Additionally, this two-color system can be used to perform a high throughput screening for opioid agonists that activate MOR but not KOR.

### Example 4

A time-gated biosensor was engineered where opioids are recorded during a specific user-defined window. The design of the time-gated biosensor is shown in FIG. 29A. “A” and “B” represent different proteins that interact. A stimulus such as a small compound or light can be used to induce a protein-protein interaction between A and B, thereby recruiting cpGFP-Nb39 to the membrane. Once recruited, an opioid will recruit Nb39 to OR, releasing cpGFP and allowing the fluorophore to mature. As proof of principle, the protein-interaction pair FKBP and FRB were used to test the system. In the presence of rapamycin (Rap), FKBP and FRB interact, brining cpGFP and Nb39 to the membrane. In the presence of opioid, Nb39 will bind to the OR, allowing the cpGFP fluorophore to mature. This sensor was designed and tested for both MOR and KOR. Results are shown in FIG. 29B.

A time-gated opioid biosensor is beneficial because it can reduce the overall background of the system and give valuable information about the temporal dynamics of opioid signaling. This system can also be used to detect the interaction between A and B with the temporal control gated by the addition of opioids. This provides an additional temporally-gated reporter for detecting protein-protein interaction (PPI). This new temporally-gated PPI system will provide an alternative for detecting PPI. When used together with the red-SPOTIT, it allows multiplexed detection of 2 pairs of PPI simultaneously.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

We claim:

1. A genetically-encoded fluorescent indicator (GEFI), comprising:
a. a circularly-permuted fluorescent protein (cpFP), and
b. an inhibitory molecule bound to the cpFP,
wherein inhibitory molecule inhibits fluorescence from the cpFP in the basal state, and wherein cpFP fluorescence is disinhibited upon conformational change of the GEFI and/or disruption of the bond between the cpFP and the inhibitory molecule.

2. The GEFI of claim 1, wherein the inhibitory molecule bound to the cpFP is a nanobody.

3. The GEFI of claim 2, wherein the nanobody comprises Nb39.

4. The GEFI of any of the preceding claims, wherein the inhibitory molecule is bound to the cpFP by a linker.

5. The GEFI of claim 4, wherein the linker comprises LKEDI (SEQ ID NO: 4).

6. The GEFI of any of the preceding claims, wherein the cpFP is bound to a protein.

7. The GEFI of claim 6, wherein the cpFP is bound to a G-protein coupled receptor (GPCR).

8. The GEFI of claim 7, wherein the cpFP is bound to the C-terminal domain of the GPCR.

9. The GEFI of any one of claims 6-8, wherein the GPCR is an opioid receptor.

10. The GEFI of claim 9, wherein the opioid receptor is a mu-opioid receptor, a kappa-opioid receptor, or a chimeric opioid receptor.

11. The GEFI of claim 10, wherein the opioid receptor is a kappa-opioid receptor comprising the amino acid sequence of SEQ ID NO: 7, SEQ ID NO: 11, or SEQ ID NO: 12.

12. The GEFI of claim 10, wherein the opioid receptor is a chimeric opioid receptor comprising the amino acid sequence of SEQ ID NO: 8.

13. The GEFI of any one of claims 6-12, wherein the cpFP is bound to the protein by a linker.

14. The GEFI of claim 13, wherein the linker comprises FPLKMRMERQGAP (SEQ ID NO: 5) or GAP.

15. The GEFI of any one of claims 1-14, wherein the GEFI comprises an amino acid sequence having at least 90% sequence identity with SEQ ID NO: 17.

16. The GEFI of claim 15, wherein the GEFI comprises the amino acid sequence of SEQ ID NO: 17.

17. The GEFI of any one of claims 1-14, wherein the GEFI comprises an amino acid sequence having at least 90% sequence identity with SEQ ID NO: 21.

18. The GEFI of any claim 17, wherein the GEFI comprises the amino acid sequence of SEQ ID NO: 21.

19. The GEFI of any one of claims 1-14, wherein the GEFI comprises an amino acid sequence having at least 90% sequence identity with SEQ ID NO: 18.

20. The GEFI of claim 19, wherein the GEFI comprises the amino acid sequence of SEQ ID NO: 18.

21. The GEFI of any one of claims 1-14, wherein the GEFI comprises an amino acid sequence having at least 90% sequence identity with SEQ ID NO: 27.

22. The GEFI of claim 21, wherein the GEFI comprises the amino acid sequence of SEQ ID NO: 27.

23. The GEFI of any one of claim 1-14, wherein the GEFI comprises an amino acid sequence having at least 90% sequence identity with SEQ ID NO: 28.

24. The GEFI of claim 23, wherein the GEFI comprises the amino acid sequence of claim 31.

25. A construct encoding the GEFI of any of the preceding claims.

26. A cell comprising the GEFI of any of the preceding claims.

27. A kit comprising the GEFI of any of the preceding claims.

28. Use of the GEFI of any of the preceding claims, the cell of claim 26, or the kit of claim 27 in a method of detecting protease activity, detecting protein-protein interaction, or detecting a G-protein coupled receptor agonist in a sample.

29. A method of determining whether an agent is a G-protein coupled receptor (GPCR) agonist, comprising:
a. providing a system containing a genetically-encoded fluorescent indicator (GEFI), wherein the GEFI comprises a G-protein coupled receptor (GPCR), a circularly-permuted fluorescent protein (cpFP), and an inhibitory molecule, wherein the C-terminal domain of the GPCR is bound to the cpFP, and wherein the cpFP is bound to the inhibitory molecule;
b. adding an agent to the system, and
c. detecting the presence or absence of a fluorescent signal after addition of the agent,
wherein the inhibitory molecule inhibits fluorescence from the cpFP in the basal state, and wherein the agent is identified as a GPCR agonist if a fluorescent signal is detected in step c).

30. The method of claim 29, wherein the system comprises a cell.

31. The method of claim 29 or 30, wherein the inhibitory molecule is a nanobody.

32. The method of claim 31, wherein the nanobody comprises Nb39.

33. The method of any one of claims 29-32, wherein the inhibitory molecule is bound to the cpFP by a linker.

34. The method of claim 33, wherein the linker comprises LKEDI (SEQ ID NO: 4).

35. The method of any one of claims 29-34, wherein the GPCR is an opioid receptor.

36. The method of claim 35, wherein the opioid receptor is a mu-opioid receptor, a kappa-opioid receptor, or a chimeric opioid receptor.

37. The method of claim 36, wherein the opioid receptor is a kappa-opioid receptor comprising the amino acid sequence of SEQ ID NO: 7, SEQ ID NO: 11, or SEQ ID NO: 12.

38. The method of claim 37, wherein the opioid receptor is a chimeric opioid receptor comprising the amino acid sequence of SEQ ID NO: 8.

39. The method of any one of claims 29-38, wherein the cpFP is bound to the GPCR by a linker.

40. The method of claim 39, wherein the linker comprises FPLKMRMERQGAP (SEQ ID NO: 5) or GAP.

41. The method of any one of claims 29-40, wherein the GEFI comprises an amino acid sequence having at least 90% sequence identity with SEQ ID NO: 17.

42. The method of claim 41, wherein the GEFI comprises the amino acid sequence of SEQ ID NO: 17.

43. The method of any one of claims 29-40, wherein the GEFI comprises an amino acid sequence having at least 90% sequence identity with SEQ ID NO: 21.

44. The method of claim 43, wherein the GEFI comprises the amino acid sequence of SEQ ID NO: 21.

45. The method of any one of claims 29-40, wherein the GEFI comprises an amino acid sequence having at least 90% sequence identity with SEQ ID NO: 18.

46. The method of claim 45, wherein the GEFI comprises the amino acid sequence of SEQ ID NO: 18.

47. The method of any one of claims 29-40, wherein the GEFI comprises an amino acid sequence having at least 90% sequence identity with SEQ ID NO: 27.

48. The method of claim 47, wherein the GEFI comprises the amino acid sequence of SEQ ID NO: 27.

49. The method of any one of claims 29-40, wherein the GEFI comprises an amino acid sequence having at least 90% sequence identity with SEQ ID NO: 28.

50. The method of claim 49, wherein the GEFI comprises the amino acid sequence of claim 31.

51. A method of evaluating a protein-protein interaction in a sample, comprising:
a. contacting a sample comprising a first protein with a genetically-encoded fluorescent indicator (GEFI), wherein the GEFI comprises a second protein, a circularly-permuted fluorescent protein (cpFP), and an inhibitory molecule, wherein the C-terminal domain of first protein is bound to the cpFP, and wherein the cpFP is bound to the inhibitory molecule; and
b. detecting a fluorescent signal in the sample,
wherein a detectable fluorescent signal in the sample indicates that a protein-protein interaction between the first protein and the second protein has occurred.

52. The method of claim 51, wherein the sample comprises a cell.

53. The method of claim 52, wherein the first protein is bound to an opioid receptor expressed by the cell.

54. The method of claim 53, further comprising contacting the sample with an opioid receptor agonist prior to detecting the fluorescent signal in the sample, wherein when a protein-protein interaction between the first protein and the second protein has occurred, the opioid receptor agonist induces a conformational change in the GEFI such that a fluorescent signal is observed.

55. The method of any one of claims 51-54, wherein the inhibitory molecule is a nanobody.

56. The method of claim 55, wherein the nanobody comprises Nb39.
